(19) United States
(12) Patent Application Publication
(10) Pub. No.: US 2022/0315969 A1
(43) Pub. Date: Oct. 6, 2022

(54) OLIVETOLIC ACID CYCLASE VARIANTS AND METHODS FOR THEIR USE

(71) Applicant: Genomatica Inc., San Diego, CA (US)

(72) Inventors: Michael Angus Noble, San Diego, CA (US); Russell Scott Komor, San Diego, CA (US)

(21) Appl. No.: 17/616,842

(22) PCT Filed: Jun. 5, 2020
(86) PCT No.: PCT/US2020/036310

§ 371 Date: Dec. 6, 2021

Related U.S. Application Data
(60) Provisional application No. 62/858,168, filed on Jun. 6, 2019.

(51) Int. Cl. C12P 17/06; C12N 1/20; C12N 15/70; C12N 15/52; C12P 7/42

(52) U.S. Cl. CPC C12P 17/06; C12N 1/20; C12N 15/70; C12Y 404/01026; C12Y 203/01206; C12N 15/52; C12P 7/42; C12N 2800/101

(57) ABSTRACT

Described herein are olivetolic acid cyclases (OAC) including non-natural variants capable of forming a 2,4-dihydroxy-6-alkylbenzoic acid from a 3,5,7-trioxoacyl-CoA or a 3,5,7-trioxocarboxylate substrate. In some examples, the non-natural OAC is capable of forming a 2,4-dihydroxy-6-alkylbenzoic acid from a 3,5,7-trioxoacyl-CoA or a 3,5,7-trioxocarboxylate substrate at a greater rate. In some examples, the non-natural OAC has a higher affinity for a 3,5,7-trioxoacyl-CoA or a 3,5,7-trioxocarboxylate substrate, as compared to the wild type OAC. The non-natural OAC can be used with olivetol synthase (OLS) to form the 2,4-dihydroxy-6-alkylbenzoic acid from malonyl-CoA and acyl-CoA through to a 3,5,7-trioxoacyl-CoAintermediate. The non-natural OAC (and OLS) can be expressed in an engineered cell having a pathway to form cannabinoids, which include CBGA, its analogs and derivatives. CBGA can be used for the preparation of cannabigerol (CBG), which can be used in therapeutic compositions.

### CROSS REFERENCE TO RELATED APPLICATION

This application claims the benefit of U.S. Provisional Patent Application Ser. No. 62/858,168 filed Jun. 6, 2019, entitled OLIVETOLIC ACID CYCLASE VARIANTS AND METHODS FOR THEIR USE, the disclosure of which is incorporated herein by reference. The entire content of the ASCII text file entitled “GNO0108WO_Sequence_Listing.txt” created on Jun. 3, 2020, having a size of 8 kilobytes is incorporated herein by reference.

### BACKGROUND

Cannabinoids constitute a varied class of chemicals that bind to cellular cannabinoid receptors. Modulation of these receptors has been associated with different types of physiological processes including pain-sensation, memory, mood, and appetite. Endocannabinoids, which occur in the body, phytocannabinoids, which are found in plants such as cannabis, and synthetic cannabinoids, can have activity on cannabinoid receptors and elicit biological responses.

Cannabis sativa produces a variety of phytocannabinoids, for example, cannabigerolic acid (CBGA), which is a precursor of tetrahydrocannabinol (THC), the primary psychoactive compound in cannabis. Additionally, CBGA is also a precursor for A9-tetrahydrocannabinolic acid (Δ⁹-THCA), cannabichromenic acid (CBCA), and Cannabidiolic acid (CBDA).

In C. sativa, precursors of CBD, CBG, CBC, and THC are carboxylic acid-containing molecules referred to as Δ⁹-tetrahydrocannabinoic acid (Δ⁹-THCA), CBDA, cannabigerolic acid (CBGA), and cannabichromenic acid (CBCA), respectively. Δ⁹-THCA, CBDA, CBGA, and CBCA are bioactive after decarboxylation, such as caused by heating, to their bioactive forms, e.g. CBGA to CBG.

Despite the well-known actions of THC, the non-psychoactive CBD, CBG, and CBC cannabinoids also have important therapeutic uses. For example, these cannabinoids can be used for the treatment of conditions and diseases that are altered or improved by action on the CB₁ and/or CB₂ cannabinoid receptors, and/or α₂-adrenergic receptor. CBG has been proposed for the treatment of glaucoma as it has been shown to relieve intraocular pressure. CBG can also be used to treat inflammatory bowel disease. Further, CBG can also inhibit the uptake of GABA in the brain, which can decrease anxiety and muscle tension.

Cannabinoids are prenylated polyketides derived from fatty acid and isoprenoid precursors. The first enzyme in the cannabinoid pathway is olivetol synthase (OLS) which is a polyketide synthase (a PKS). OLS catalyzes the condensation of hexanoyl-CoA with three molecules of malonyl-CoA to yield 3,5,7-trioxododecanoyl-CoA (see FIG. 1). Olivetol synthase has also been termed “tetraketide synthase” (TKS) based on its role in the cannabinoid pathway (Gagne et al., PNAS, 109: 12811-12816).

The intermediate 3,5,7-trioxododecanoyl-CoA is then converted to olivetolic acid (OLA) by the enzyme olivetolic acid cyclase (Gagne et al., PNAS, 109: 12811-12816), referred to as “OAC”. As noted in Gagne et al., OAC is a dimeric α+β barrel (DABB) protein that is structurally similar to DABB-type polyketide cyclase enzymes from Streptomyces and to stress-responsive proteins in plants. In Yang et al. (FEBS Journal 283:1088-1106; 2016) the OAC apo and OAC-OLA complex binary crystal structures were solved at 1.32 and 1.70 Å resolutions, respectively. The crystal structures confirmed OAC belongs to the DABB superfamily, and possesses a unique active-site cavity containing the pentyl-binding hydrophobic pocket and the polyketide binding site. Yang et al. proposes that OAC employs unique catalytic machinery utilizing acid/base catalytic chemistry for formation of OLA precursor.

OLA is then prenylated by an aromatic prenyltransferase, which adds a partially saturated carbon chain to a carbon position on the OLA hydroxylated and carboxylated ring. The partially saturated carbon chain is provided by the substrate geranyl pyrophosphate (GPP).

The addition of the partially saturated carbon chain from GPP to OLA forms cannabigerolic acid (CBGA), which is a common precursor to cannabinoids.

### SUMMARY

Aspects of the disclosure are directed towards non-natural olivetolic acid cyclases (OACs) that include at least one amino acid variation that differs from an amino acid residue of a wild type olivetolic acid cyclase, engineered cells comprising the non-natural OACs, and methods of using the non-natural OACs and the engineered cells to produce desired compounds.

In embodiments, the OAC enzyme is a homodimeric protein, with each subunit having the same amino acid residues. Although the amino acid sequences of the monomers are same, significant conformational differences between OAC monomers A and B were observed during the three-dimensional structure analysis. In other embodiments, the OAC enzyme is a heterodimeric protein, with each subunit having of different amino acid residues.

Non-natural OACs of the disclosure are capable of producing hydroxylated and alkylated benzoic acid precursors which can be used for the formation of prenylated aromatic compounds, including cannabinoids, and cannabinoid analogs and derivatives thereof. In aspects, non-natural OACs of the disclosure are engineered to provide one or more non-natural amino acids (i.e., variant amino acid(s)) in proximity of the active site of the OAC, wherein the variant amino acid(s) accommodate chemical deviations in the 3,5,7-trioxoacyl-CoA or a 3,5,7-trioxocarboxylate substrate and in turn provide improved catalytic activity and/or affinity for the particular substrate.

As described herein, OAC variants can be designed to provide improved catalytic activity and/or affinity for 3,5,7-trioxoacyl-CoA or a 3,5,7-trioxocarboxylate substrates that are larger and more hydrophobic than 3,5,7-trioxododecanoyl-CoA or 3,5,7-trioxododecanoate, that are smaller and less hydrophobic than 3,5,7-trioxododecanoyl-CoA or 3,5,7-trioxododecanoate, or that are more polar and/or more charged than 3,5,7-trioxododecanoyl-CoA or 3,5,7-trioxododecanoate. Experimental studies associated with the current disclosure include structural analysis of analogs of a 3,5,7-trioxododecanoyl-CoA linear tetraketide substrate docked into the OAC active site and have identified various catalytically relevant substrate binding conformations for the linear tetraketide substrate. In turn, the structural analysis has allowed for identification of amino acids within a certain radius of the site that can be modified to accommodate changes in the chemical structure of the linear tetraketide substrate. As described herein, catalytically relevant amino acid residues having bulky or large hydrophobic side chains can be modified to those having smaller, less bulky hydrophobic side chains in the non-natural OAC to accommodate linear tetraketide substrates that have alkyl chains larger than the pentyl chain of 3,5,7-trioxododecanoyl-CoA. Conversely, catalytically relevant amino acid residues having small or less bulky hydrophobic side can be modified to those having larger, more bulky hydrophobic side chains alkyl chains smaller than the pentyl chain. Also, any chemical charge changes to the linear tetraketide can be accommodated by altering the relevant side chain(s) in the non-natural OAC to provide introduce a corresponding, opposite, charge.

In turn, engineered cells including OAC variants of the disclosure can effectively utilize various 3,5,7-trioxoacyl-CoA or a 3,5,7-trioxocarboxylate substrates to form desired 2,4-dihydroxy-6-alkylbenzoic acids, which in turn can be used as substrates for forming different types of cannabinoid analogs and derivatives thereof.

In one aspect, provided is a non-natural olivetolic acid cyclase comprising at least one amino acid variation as compared to a wild type OAC. The non-natural OAC is enzymatically capable of: a) forming olivetolic acid from a 3,5,7-trioxododecanoyl-CoA or a 3,5,7-trioxododecanoate substrate; b) forming olivetolic acid from a 3,5,7-trioxododecanoyl-CoA or a 3,5,7-trioxododecanoate substrate at a greater rate as compared to the wild type OAC; (c) having a higher affinity for a 3,5,7-trioxododecanoyl-CoA or a 3,5,7-trioxododecanoate substrate as compared to the wild type OAC; (d) with wild-type or non-natural OLS, forming olivetolic acid from malonyl-CoA and hexanoyl-CoA through a 3,5,7-trioxododecanoyl-CoA or a 3,5,7-trioxododecanoate intermediate at a greater rate as compared to the wild type OAC, or (e) any combination of a), b), c), and d); with the proviso that the non-natural OAC does not have a single mutation of Y27F relative to SEQ ID NO:1. OLS and OAC can function cooperatively to synthesize a 3,5,7-trioxododecanoyl-CoA or a 3,5,7-trioxododecanoate intermediate from malonyl-CoA and hexanoyl-CoA substrates and then cyclize the 3,5,7-trioxododecanoyl-CoA or a 3,5,7-trioxododecanoate intermediate to form olivetolic acid.

In one aspect, provided is a non-natural olivetolic acid cyclase comprising at least one amino acid variation as compared to a wild type OAC. The non-natural OAC is enzymatically capable of: a) forming a 2,4-dihydroxy-6-alkylbenzoic acid from a 3,5,7-trioxoacyl-CoA or a 3,5,7-trioxocarboxylate substrate; b) forming a 2,4-dihydroxy-6-alkylbenzoic acid from a 3,5,7-trioxoacyl-CoA or a 3,5,7-trioxocarboxylate substrate at a greater rate as compared to the wild type OAC; (c) having a higher affinity for a 3,5,7-trioxoacyl-CoA or a 3,5,7-trioxocarboxylate substrate as compared to the wild type OAC; (d) with wild-type or non-natural OLS, forming a 2,4-dihydroxy-6-alkylbenzoic acid from malonyl-CoA and acyl-CoA through a 3,5,7-trioxoacyl-CoA or a 3,5,7-trioxocarboxylate intermediate at a greater rate as compared to the wild type OAC, or (e) any combination of a), b), c), and d); with the proviso that the non-natural OAC does not have a single mutation of Y27F relative to SEQ ID NO:1. OLS and OAC can function cooperatively to synthesize a 3,5,7-trioxoacyl-CoA product from acyl-CoA substrates and then cyclize the 3,5,7-trioxoacyl-CoA product to form a 2,4-dihydroxy-6-alkylbenzoic acid.

In one aspect, provided are a non-natural OAC comprising one or more amino acid variations at position(s) selected from the group consisting of H5X¹, wherein X¹ is selected from the group consisting of G,A,C,P,V,L,I,M,F,Y,W, Q,E,K,R,S,T,Y,N,Q,D,E,K, and R; I7X², wherein X² is selected from the group consisting of G,A,C,P,V,L,M,FY,W,K,R,S,T,H,N,Q,D, and E; L9X³, wherein X³ is selected from the group consisting of G,A,C,P,V,I,M,F,Y,W,K,R,S,T,Y,H,N,Q,D, E,K,R; F23X⁴, wherein X⁴ is selected from the group consisting of G,A,C,P,V,L, I,M,Y,W,S,T,H,N,Q,D,E,K, and R; F24X⁵, wherein X⁵ is selected from the group consisting of G,A,C,P,V,I,M,Y,S,T,H,N,Q,D,E,K,R, and W; Y27X6, wherein X6 is selected.from the group consisting of G,A,C,P,V,L,I,M,F,W,S,T,H,N,Q,D,E,K, and R; V59X⁷, wherein X⁷ is selected from the group consisting of G,A,C,P,L,I,M,F,Y, W,H,Q,E,K, and R; V61X⁸, wherein X⁸ is selected from the group consisting of G,A,C,P,L,I,M,F,Y,W,H,Q,E,K,R,S,T,N, and D; V66X⁹, wherein X⁹ is selected from the group consisting of G,A,C,P,L,I,M,F,Y, and W; E67X¹⁰, wherein X¹⁰ is selected from the group consisting of G,A,C,P,V,L,I,M,F,Y, and W; I69X¹¹, wherein X¹¹ is selected from the group consisting of G,A,C,P,V,L,M,F,Y, and W; Q70X¹², wherein X² is selected from the group consisting of S,T,H,N,D,E,R,K, and Y; 173X¹³, wherein X¹³ is selected from the group consisting of G,A,C,P,V,L,M,F,Y, and W; I74X¹⁴, wherein X¹⁴ is selected from the group consisting of G,A,C,P,V,L, M,F,Y, and W; V79X¹⁵, wherein X⁵ is selected from the group consisting of G,A,C,P,L,I,M,F,Y, and W; G80X¹⁶, wherein X¹⁶ is selected from the group consisting of A,C,P,V,L,I,M,F,Y,W,S,T,H,N,Q,D,E,K, and R; F81X⁷, wherein X¹⁷ is selected from the group consisting of G,A,C,P,V,L,I,M,Y,W,S,T,H,N,Q,D,E,R, and K; G82X¹⁸, wherein X¹⁸ is selected from the group consisting of A,C,P,V,L,I,M, F,Y,W,S,T,H,N,Q,E,K, and R; D83X¹⁹, wherein X¹⁹ is selected from the group consisting of S,T,H,Q,N,E,R,K, and Y; R86X²⁰, wherein X²⁰ is selected from the group consisting of S,T,H,Q,N,D,E,K, and Y; W89X², wherein X²¹ is selected from the group consisting of G,A,C,P,V,L,I,M,F,Y,W,S,T,H,N,Q,D,E,K, and R; L92X²², wherein: X² is selected from the group consisting of G,A,C,P,V,I,M,F,Y, and W; I94X²³, wherein X²³ is selected from the group consisting of G,A,C,P,V,L,M,F,Y,W,K,R,S,T,Y,H,N,Q,D, and E; D96X²⁴, wherein X²⁴ is selected from the group consisting of S,T,H,Q,N,E,R,K, and Y; V46X², wherein X² is selected from the group consisting of G,A,C,P,L,I,M,F,Y, and W; T47X²⁶, wherein X²⁶ is selected from the group consisting of S,H,Q,N,D,E,R,K, and Y; Q48X², wherein X² is selected from the group consisting of S,T,H,N,D,E,R,K, and Y; K49X²⁸, wherein X²⁸ is selected from the group consisting of S,T,H,Q,N,D,E,R, and Y; N50X²⁹, wherein X²⁹ is selected from the group consisting of G,A,C,P,V,L, I,M,F,Y, and W; and K51X³⁰, wherein X³⁰ is selected from the group consisting of S,T,H,Q,N,D,E,R, and Y, wherein the amino acid positions correspond to SEQ ID NO: 1, and wherein the non-natural OAC is not a single variant of K4A, H5A, H5L, H5Q, H5S, H5N, H5D, I7L, I7F, L9A, L9W, K12A, F23A, F23I, F23W, F23L, F24L, F24W, F24A, Y27F, Y27M, Y27W, V28F, V29M, K38A, V40F, D45A, H57A, V59M, V59A, V59F, Y72F, H75A, H78A, H78N, H78Q, H78S, H78D, or D96A.

In one aspect, provided is a non-natural OAC comprising one or more amino acid variations at position(s) selected from the group consisting of H5X¹, wherein X¹ is selected from the group consisting of G,A,C,P,V,L,I,M,F,Y,W, Q,E,K,R,S,T,Y,N,Q,D,E,K, and R; I7X², wherein X² is selected from the group consisting of G,A,C,P,V,L,M,FY,W,K,R,S,T,H,N,Q,D, and E; L9X³, wherein X³ is selected from the group consisting of G,A,C,P,V,I,M,F,Y,W,K,R,S,T,Y,H,N,Q,D,E, K,R; F23X⁴, wherein X⁴ is selected from the group consisting of G,A,C,P,V,L, I,M, Y,W,S,T,H,N,Q,D,E,K, and R; F24X⁵, wherein X⁵ is selected from the group consisting of G,A,C,P, V,I,M,Y,S,T,H,N,Q,D,E,K,R, and W; Y27X⁶, wherein X⁶ is selected from the group consisting of G,A,C,P,V,L,I,M,F,W,S,T,H,N,Q,D,E,K, and R; V59X⁷, wherein X⁷ is selected from the group consisting of G,A,C,P,L,I,M,F,Y, W,H,Q,E,K, and R; V61X⁸, wherein X⁸ is selected from the group consisting of G,A,C, P,L,I,M,F,Y,W,H,Q,E,K,R,S,T,N, and D; V66X⁹, wherein X⁹ is selected from the group consisting of G, A,C,P,L,I,M,F,Y, and W; E67X¹⁰, wherein X¹⁰ is selected from the group consisting of G,A,C,P,V,L,I,M,F,Y, and W; I69X¹¹, wherein X¹¹ is selected from the group consisting of G,A,C,P,V,L,M,F,Y, and W; Q70X¹², wherein X² is selected from the group consisting of S,T,H,N,D,E,R,K, and Y; I73X¹³, wherein X¹³ is selected from the group consisting of G,A,C,P,V,L,M,F,Y, and W; I74X¹⁴, wherein X¹⁴ is selected from the group consisting of G,A,C,P,V,L, M,F,Y, and W; V79X¹, wherein X¹ is selected from the group consisting of G,A,C, P,L,I,M,F,Y, and W; G80X¹⁶, wherein X¹⁶ is selected from the group consisting of A,C,P,V,L,I,M,F,Y,W,S,T,H,N,Q,D,E,K, and R; F81X¹⁷, wherein X¹⁷ is selected from the group consisting of G,A,C,P,V,L,I,M,Y,W,S,T,H,N,Q,D,E,R, and K; G82X¹⁸, wherein X¹⁸ is selected from the group consisting of A,C,P,V,L,I,M,F,Y,W, S,T,H,N,Q,E,K, and R; D83X¹⁹, wherein X¹⁹ is selected from the group consisting of S,T,H,Q,N,E,R,K, and Y; R86X²⁰, wherein X²⁰ is selected from the group consisting of S,T,H,Q,N,D,E,K, and Y; W89X²¹, wherein X²¹ is selected from the group consisting of G,A,C,P,V,L,I,M,F,Y,W,S,T,H,N,Q,D,E,K, and R; L92X²² wherein X²² is selected from the group consisting of G,A,C,P,V,I,M,F,Y, and W; I94X²³, wherein X² is selected from the group consisting of G,A,C,P,V,L,M,F,Y,W, K,R,S,T,Y,H,N,Q,D, and E; D96X²⁴, wherein X²⁴ is selected from the group consisting of S,T,H,Q,N,E,R,K, and Y; V46*X²⁵, wherein X²⁵ is selected from the group consisting of G,A,C,P,L,I,M,F,Y, and W; T47*X²⁶, wherein X²⁶ is selected from the group consisting of S,H,Q,N,D,E,R,K, and Y; Q48*X²⁷, wherein X²⁷ is selected from the group consisting of S,T,H,N,D,E,R,K, and Y; K49*X²⁸, wherein X²⁸ is selected from the group consisting of S,T,H,Q,N, D,E,R, and Y; N50*X²⁹, wherein X²⁹ is selected from the group consisting of G,A,C,P,V,L, I,M,F,Y, and W; and K51*X³⁰, wherein X³⁰ is selected from the group consisting of S,T,H,Q,N,D,E, R, and Y, wherein the amino acid positions correspond to SEQ ID NO: 1, and wherein the non-natural OAC is not a single variant of K4A, HSA, H5L, H5Q, H5S, H5N, H5D, 17L, 17F, L9A, L9W, K12A, F23A, F23I, F23W, F23L, F24L, F24W, F24A, Y27F, Y27M, Y27W, V28F, V29M, K38A, V40F, D45A, H57A, V59M, V59A, V59F, Y72F, H75A, H78A, H78N, H78Q, H78S, H78D, or D96A, and wherein the “*” indicates amino acid residues from chain B of OAC dimer and corresponding to SEQ ID NO: 1.

In some embodiments, the amino acid sequence of the non-natural olivetolic acid cyclase is at least 45%, 50%, 55%, 60%, 65%, 70%, 75%, 80%, 85%, 90%, 95%, 99%, or 100% identical to at least 25, at least 30, at least 35, at least 40, at least 45, at least 50, at least 55, at least 60, at least 75, at least 80, at least 85, at least 90, or at least 95 contiguous amino acids of any of SEQ ID NOs:1-3. In some embodiments, the non-natural OAC comprises at least two, three, four, five, six, seven, eight, nine, or more amino acid variations as compared to a wild type OAC. In some embodiments, the amino acid sequence of the non-natural olivetolic acid cyclase comprises SEQ ID Nos: 1-3. In some embodiments, the amino acid sequence of the non-natural olivetolic acid cyclase is SEQ ID NO: 2.

In some embodiments, the disclosure provides a non-natural OAC having one or more amino acid variations at the following locations relative to SEQ ID NO:1 or an OAC template having at least 60% identity to SEQ ID NO:1 or to at least 25 contiguous amino acids of SEQ ID NO:1 and comprises a amino acid substitution at positions H5, I7, L9, F23, F24, Y27, V59, V61, V66, E67, I69, Q70, I73, I74, V79, G80, F81, G82, D83, R86, W89, L92, I94, D96, V46, T47, Q48, K49, N50, and K51.

In some embodiments, the disclosure provides a non-natural OAC having one or more amino acid variations at the following locations relative to SEQ ID NO:1 or an OAC template having at least 60% identity to SEQ ID NO:1 or to at least 25 contiguous amino acids of SEQ ID NO:1 and comprises a amino acid substitution at positions H5, I7, L9, F23, F24, Y27, V59, V61, V66, E67, I69, Q70, I73, I74, V79, G80, F81, G82, D83, R86, W89, L92, I94, D96, V46*, T47*, Q48*, K49*, N50*, and K51* corresponding to SEQ ID NO: 1, wherein the “*” indicates residues from chain B of OAC dimer.

In one aspect, provided are non-natural OAC having a higher affinity for a 3,5,7-trioxoacyl-CoA or a 3,5,7-trioxocarboxylate substrate that is different than 3,5,7 trioxododecanoyl-CoA, as compared to the wild type OAC., and/or that is able to form a 3,5,7-trioxoacyl-CoA or a 3,5,7-trioxocarboxylate substrate that is different than 3,5,7 trioxododecanoyl-CoA at a greater rate as compared to the wild type OAC.

In one aspect, provided are non-natural OAC having a higher affinity for a hydrophobic 3,5,7-trioxoacyl-CoA or a 3,5,7-trioxocarboxylate substrate that is larger than 3,5,7 trioxododecanoyl-CoA, comprising one or more amino acid variations at position(s): H5X¹, wherein X¹ is selected from the group consisting of G,A,C,P,V; I7X², wherein X² is selected from the group consisting of G,A,C,P,V,L, and M; L9X³, wherein X³ is selected from the group consisting of G,A,C,P,V,I, and M; F23X⁴, wherein X⁴ is selected from the group consisting of G,A,C,P,V,L,I,M,Y, W,S,T,H,N,Q,D,E,K, and R; F24X⁵, wherein X⁵ is selected from the group consisting of G,A,C,P,V,L,I,M,Y,W,S,T, H,N,Q,D,E,K, and R; Y27X⁶, wherein X⁶ is selected from the group consisting of G,A,C,P,V,L,I,M,F,W,S,T,H,N,Q,D,E,K, and R; V59X⁷, wherein X⁷ is selected from the group consisting of G,A,C, and P; V61X⁸, wherein X⁸ is selected from the group consisting of G,A,C, and P; G80X¹⁶, wherein X¹⁶ is selected from the group consisting of A,C,P,V,L,I,M,F,Y,W,S,T,H,N, Q,D,E,K, and R; F81X¹⁷, wherein X¹⁷ is selected from the group consisting of Y and W; G82X¹⁸, wherein X¹⁸ is selected from the group consisting of A,C,P,V,L,I, M,F,Y,W,S,T,H,N,Q,D,E,K, and R; W89X²¹, wherein X²¹ is selected from the group consisting of G,A,C,P,V,L,I,M,F,Y,W,S,T,H,N,Q,D,E,K, and R; L92X², wherein X²² is selected from the group consisting of G,A,C,P;V,I, and M; and 94X²³ wherein X²³ is selected from the group consisting of G,A,C,P,V,L, and M, wherein the amino acid positions correspond to SEQ ID NO: 1.

In one aspect, provided are non-natural OAC having a higher affinity for a hydrophobic 3,5,7-trioxoacyl-CoA or a 3,5,7-trioxocarboxylate substrate that is smaller than 3,5,7-trioxododecanoyl-CoA or a 3,5,7-trioxododecanoate substrate, comprising one or more amino acid variations at position(s): H5X¹, wherein X¹ is selected from the group consisting of V,M,F,Y,W,Q,E, and K, and R; I7X², wherein X² is selected from the group consisting of L,M,F,Y,W,K, and R; L9X³, wherein X³ is selected from the group consisting of I,M,F,Y,W,K, and R; F23X⁴, wherein X⁴ is selected from the group consisting of Y and W; F24X⁵, wherein X⁵ is selected from the group consisting of Y and W; Y27X⁶, wherein X⁶ is selected from the group consisting of F and W; V59X⁷, wherein X⁷ is selected from the group consisting of M,F,Y,W,H,Q,E,K, and R; V61X⁸, wherein X is selected from the group consisting of M,F,Y,W,H,Q,E,K, and R; G80X¹⁶, wherein X¹⁶ is selected from the group consisting of A,C,P, and V; F81X¹⁷, wherein X¹⁷ is selected from the group consisting of G,A,C,P,V,L,I,M,Y,W,S,T,H,N,Q,D,E,K, and R; G82X¹⁸, wherein X¹⁸ is selected from the group consisting of A,C,P, and V; W89X²¹, wherein X²¹ is selected from the group consisting of F, and Y; L92X²², wherein X²² is selected from the group consisting of I,M,F,Y,W,K, and R; and I94X², wherein X² is selected from the group consisting of L,M,F,Y,W,K, and R, wherein the amino acid positions correspond to SEQ ID NO: 1.

In one aspect, provided are non-natural OAC having a higher affinity for a 3,5,7-trioxoacyl-CoA or a 3,5,7-trioxocarboxylate substrate that is more polar and/or more charged than 3,5,7 trioxododecanoyl-CoA, comprising one or more amino acid variations at position(s): H5X¹, wherein X¹ is selected from the group consisting of S,T,Y,N,Q,D,E,K, and R; I7X², wherein X² is selected from the group consisting of S,T,Y,H,N,Q,D,E,K, and R; L9X³, wherein X³ is selected from the group consisting of S,T,Y,H, N,Q,D,E,K, and R; F23X⁴, wherein X⁴ is selected from the group consisting of S,T,Y,H,N,Q,D,E,K, and R; F24X⁵, wherein X⁵ is selected from the group consisting of S,T,Y,H,N,Q,D,E,K, and R; Y27X⁶, wherein X⁶ is selected from the group consisting of S,T,H,N,Q,D,E,K, and R; V59X⁷, wherein X⁷ is selected from the group consisting of S,T,Y,H,N,Q,D,E,K, and R; V61X⁸, wherein X⁸ is selected from the group consisting of S,T,Y,H,N,Q,D,E,K, and R; G80X¹⁶, wherein X¹⁶ is selected from the group consisting of S,T,Y,H,N,Q,D,E,K, and R; F81X¹⁷, wherein X¹ is selected from the group consisting of S,T,Y,H,N,Q,D,E,K, and R; G82X¹⁸, wherein X¹¹ is selected from the group consisting of S,T,Y,H,N,Q,D,E,K, and R; W89X²¹, wherein X²¹ is selected from the group consisting of S,T,Y,H,N,Q, D,E,K, and R; L92X²², wherein X²¹ is selected from the group consisting of S,T,Y, H,N,Q,D,E,K, and R; and I94X²³, wherein X²³ is selected from the group consisting of S,T,Y,H,N,Q,D,E,K, and R, wherein the amino acid positions correspond to SEQ ID NO: 1.

In some embodiments, 3,5,7-trioxoacyl-CoA or a 3,5,7-trioxocarboxylate substrates that are more polar and/or more charged than 3,5,7 trioxododecanoyl-CoA are larger than 3,5,7 trioxododecanoyl-CoA. In some embodiments, 3,5,7-trioxoacyl-CoA or a 3,5,7-trioxocarboxylate substrates that are more polar and/or more charged than 3,5,7 trioxododecanoyl-CoA are smaller than 3,5,7 trioxododecanoyl-CoA.

In some embodiments, all or portion of the OAC protein and all or portion of the OLS protein are part of the same polypeptide. In some embodiments, the OAC and/or OLS are non-natural proteins. In some embodiments, the OAC or a fragment thereof is fused with OLS or a fragment thereof. In some embodiments, the OAC protein is fused with the OLS protein through a linker molecule. In some embodiments, the N-terminus of the OAC protein or a fragment thereof is fused with the N-terminus of the OLS protein or its fragment. In some embodiments, the C-terminus of the OAC protein or a fragment thereof is fused with the C-terminus of the OLS protein or its fragment. In some embodiments, the N-terminus of the OAC protein or a fragment thereof is fused with the C-terminus of the OLS protein or its fragment. In some embodiments, the C-terminus of the OAC protein or fragment thereof is fused with the N-terminus of the OLS protein or its fragment.

In some embodiments, the OLS is a non-natural OLS. In some embodiments, the amino acid sequence of OLS is at least 60% identical to at least 25 or more contiguous amino acids of SEQ ID NO: 4. In some embodiments, (a) the amino acid sequence of the non-natural OLS comprises one or more amino acid substitutions at position(s) selected from the group consisting of: Q82S, P131A, I186F, M187E, M187N, M187T, M187I, M187S, M187A, M187L, M187G, M187V, M187C, S195K, S195M, S195R, S197G, S197V, T239E, K314D, and K314M, corresponding to the amino acid positions of SEQ ID NO:4; (b) the amino acid sequence of the non-natural OLS comprises two, or more than two amino acid substitutions, selected from: (i) Q82S and P131A, (ii) Q82S and M187S, (iii) Q82S and S195K, (iv) Q82S and S195M, (v) Q82S and S197V, (vi) Q82S and K314D, (vii) P131A and I186F, (viii) P131A and M187S, (ix) P131A and S195M, (x) P131A and S197V, (xi) P131A and K314D, (xii) P131A and K314M, (xiii) I186F and M187S, (xiv) I186F and S195K, (xv) I186F and S195M, (xvi) I186F and T239E, (xvii) I186F and K314D, (xviii) M187S and S195K, (xix) M187S and S195M, (xx) M187S and S197V, (xxi) M187S and T239E, (xxii) M187S and K314D, (xxiii) M187S and K314M, (xxiv) S195K and S197V, (xxv) S195M and S197V, (xxvi) S195M and T239E, (xxvii) S195K and K314D, (xxviii) S195K and K314M, (xxix) S195M and K314D, (xxx) S195M and K314M, (xxxi) S197V and T239E, (xxxii) S197V and K314M, (xxxiii) T239E and K314D, (xxxiv) T239E and K314M, (xxxv) Q82S and I186F, (xxxvi) Q82S and T239E, (xxxvii) Q82S and K314M, (xxxviii) I186F and S197V (xxxix) I186F and K314M, (xl) S195K and T239E, (xli) S197V and K314D, (xlii) P131A and T239E, and (xliii) P131A and S195K; or (c) the amino acid sequence of the non-natural OLS comprises three, or more than three amino acid substitutions, selected from: (i) Q82S, P131A, and I186F, (ii) Q82S, P131A, and M187S, (iii) Q82S, P131A, and S195K, (iv) Q82S, P131A, and S195M, (v) Q82S, P131A, and S197V, (vi) Q82S, P131A, and T239E, (vii) Q82S, P131A, and K314D, (viii) Q82S, P131A, and K314M, (ix) Q82S, I186F, and M187S, (x) Q82S, I186F, and S195M, (xi) Q82S, I186F, and S197V, (xii) Q82S, I186F, and T239E, (xiii) Q82S, I186F, and K314D, (xiv) Q82S, I186F, and K314M, (xv) Q82S, M187S, and S195K, (xvi) Q82S, M187S, and S195M, (xvii) Q82S, M187S, and S197V, (xviii) Q82S, M187S, and T239E, (xix) Q82S, M187S, and K314D, (xx) Q82S, M187S, and K314M, (xxi) Q82S, S195K, and S197V, (xxii) Q82S, S195M, and S197V, (xxiii) Q82S, S195K, and K314D, (xxiv) Q82S, S195K, and K314M, (xxv) Q82S, S195M, and K314D, (xxvi) Q82S, S195M, and K314M, (xxvii) Q82S, S197V, and T239E, (xxviii) Q82S, S197V, and K314D, (xxix) Q82S, S197V, and K314M, (xxx) Q82S, T239E, and K314D, (xxxi) Q82S, T239E, and K314M, (xxxii) P131A, I186F, and M187S, (xxxiii) P131A, I186F, and S195K, (xxxiv) P131A, I186F, and S195M, (xxxv) P131A, I186F, and S197V, (xxxvi) P131A, I186F, and K314D, (xxxvii) P131A, I186F, and K314M, (xxxviii) P131A, M187S, and S195K, (xxxix) P131A, M187S, and S195M, (xl) P131A, M187S, and S197V, (xli) P131A, M187S, and T239E, (xlii) P131A, M187S, and K314D, (xliii) P131A, S195M, and S197V, (xliv) P131A, S195M, and T239E, (xlv) P131A, S195K, and K314D, (xlvi) P131A, S195K, and K314M, (xlvii) P131A, S195M, and K314D, (xlviii) P131A, S195M, and K314M, (xlix) P131A, S197V, and T239E, (1) P131A, S197V, and K314D, (li) P131A, S197V, and K314M, (lii) P131A, T239E, and K314D, (liii) P131A, T239E, and K314M, (liv) I186F, M187S, and S195K, (lv) I186F, M187S, and S195M, (lvi) I186F, M187S, and S197V, (lvii) I186F, M187S, and K314M, (lviii) I186F, S195K, and S197V, (lix) I186F, S195M, and S197V, (lx) I186F, S195K, and T239E, (lxi) I186F, S195M, and T239E, (lxii) I186F, S195K, and K314D, (lxiii) I186F, S195K, and K314M, (lxiv) I186F, S195M, and K314D, (lxv) I186F, S195M, and K314M, (lxvi) I186F, S197V, and T239E, (lxvii) I186F, S197V, and K314D, (lxviii) I186F, S197V, and K314M, (lxix) I186F, T239E, and K314M, (lxx) M187S, S195K, and S197V, (lxxi) M187S, S195M, and S197V, (lxxii) M187S, S195K, and T239E, (lxxiii) M187S, S195M, and T239E, (lxxiv) M187S, S195K, and K314D, (lxxv) M187S, S195K, and K314M, (lxxvi) M187S, S195M, and K314D, (lxxvii) M187S, S195M, and K314M, (lxxviii) M187S, S197V, and T239E, (lxxix) M187S, S197V, and K314D, (lxxx) M187S, S197V, and K314M, (lxxxi) M187S, T239E, and K314D, (lxxxii) M187S, T239E, and K314M, (lxxxiii) S195K, S197V, and T239E, (lxxxiv) S195M, S197V, and T239E, (lxxxv) S195K, S197V, and K314D, (lxxxvi) S195K, S197V, and K314M, (lxxxvii) S195M, S197V, and K314D, (lxxxviii) S195M, S197V, and K314M, (lxxxix) S195K, T239E, and K314D, (xc) S195K, T239E, and K314M, (xci) S195M, T239E, and K314D, (xcii) S195M, T239E, and K314M, and (xciii) S197V, T239E, and K314M corresponding to the amino acid positions of SEQ ID NO: 4.

In some embodiments, the 3,5,7-trioxoacyl-CoA or a 3,5,7-trioxocarboxylate substrate is formed by olivetolic acid synthase from malonyl-CoA and a starter CoA. In some embodiments, the starter CoA molecules can be an acyl-CoA, aminoacyl-CoA (e.g., 2-aminoacetyl CoA, 3-aminopropionyl-CoA, 2-aminopropionyl-CoA, 4-aminobutyryl-CoA), hydroxyacyl-CoA (e.g., 2-hydroxypropionoyl-CoA, 3-hydroxybutyryl-CoA, hydroxyacetyl-CoA, hydroxypropionoyl-CoA, hydroxybutyryl-CoA), branched chain acyl-CoA (e.g., isobutyryl-CoA, 3-methylbutyryl-CoA), an aromatic acid CoA, for example, benzoic, chorismic, phenylacetic and phenoxyacetic acid CoA. Exemplary acyl-CoA include acetyl-CoA, propionyl-CoA, butyryl-CoA, valeryl-CoA, hexanoyl-CoA, heptanoyl-CoA, octanoyl-CoA, nonanoyl-CoA, decanoyl-CoA, one or more of C12, C14, C16, C18, C20 or C22 chain length fatty acid CoA. Chemical formulas for exemplary starter CoA molecules are shown in FIGS. 2 and 4.

In some embodiments, the 3,5,7-trioxoacyl-CoA or a 3,5,7-trioxocarboxylate substrate for the non-natural OAC is 3,5,7-trioxododecyl-CoA or 3,5,7-trioxododecanoate, and wherein the 2,4-dihydroxy-6-alkylbenzoic acid is olivetolic acid.

In some embodiments, the non-natural OAC is enzymatically capable of forming olivetolic acid, its analogs and derivatives or a combination thereof at a rate of least two-fold greater as compared to the rate with wild type OAC forms the same product. In some embodiments, the OAC is enzymatically capable of forming olivetolic acid, its analogs and derivatives, or a combination thereof from malonyl-CoA and an acyl-CoA in the presence of non-rate limiting amount of OLS at a rate of least two-fold greater as compared to the rate with wild type OAC forms the same product.

In another aspect, provided are nucleic acids that encode a non-natural olivetolic acid cyclase comprising at least one amino acid variation as compared to a wild type OAC. The nucleic acids encode a non-natural OAC that is enzymatically capable of: (a) forming a 2,4-dihydroxy-6-alkylbenzoic acid from a 3,5,7-trioxoacyl-CoA or a 3,5,7-trioxocarboxylate substrate; (b) forming a 2,4-dihydroxy-6-alkylbenzoic acid from a 3,5,7-trioxoacyl-CoA or a 3,5,7-trioxocarboxylate substrate at a greater rate as compared to the wild type OAC; (c) having a higher affinity for a 3,5,7-trioxoacyl-CoA or a 3,5,7-trioxocarboxylate substrate as compared to the wild type OAC; (d) with OLS, forming a 2,4-dihydroxy-6-alkylbenzoic acid from malonyl-CoA and acyl-CoA through a 3,5,7-trioxoacyl-CoA or a 3,5,7-trioxocarboxylate intermediate at a greater rate as compared to the wild type OAC, or any combination of (a), (b), (c), and (d), with the proviso that the non-natural OAC does not have a single mutation of Y27F relative to SEQ ID NO:1.

In some embodiments, the nucleic acid encoding a non-natural olivetolic acid cyclase is operably linked to a regulatory element, wherein the regulatory element is heterologous to the OAC. In some embodiments, the regulatory element is a promoter, enhancer, or a 5′-untranslated region.

In another aspect, provided are engineered cells comprising a non-natural olivetolic acid cyclase comprising at least one amino acid variation as compared to a wild type OAC, wherein the non-natural OAC is enzymatically capable of: (a) forming a 2,4-dihydroxy-6-alkylbenzoic acid from a 3,5,7-trioxoacyl-CoA or a 3,5,7-trioxocarboxylate substrate; (b) forming a 2,4-dihydroxy-6-alkylbenzoic acid from a 3,5,7-trioxoacyl-CoA or a 3,5,7-trioxocarboxylate substrate at a greater rate as compared to the wild type OAC; (c) having a higher affinity for a 3,5,7-trioxoacyl-CoA or a 3,5,7-trioxocarboxylate substrate as compared to the wild type OAC; (d) with non-rate limiting amount of OLS, forming a 2,4-dihydroxy-6-alkylbenzoic acid from malonyl-CoA and acyl-CoA through a 3,5,7-trioxoacyl-CoA or a 3,5,7-trioxocarboxylate intermediate at a greater rate as compared to the wild type OAC, or any combination of (a), (b), (c) and (d), with the proviso that the non-natural OAC does not have a single mutation of Y27F relative to SEQ ID NO:1.

An engineered cell can include one or more copies of a gene encoding the non-natural OAC. Optionally the engineered cell can include at least one copy of a gene encoding the non-natural OAC and at least one copy of a gene encoding a different OAC, for example, a wild type OAC, or a different (second) non-natural OAC with an amino acid variation that is different than the first non-natural OAC.

In some embodiments, the amino acid sequence of OAC of the engineered cell is at least 60%, 65%, 70%, 75%, 80%, 85%, 90%, 95%, 99%, or identical to any one of SEQ ID NOs: 1-3 or to at least 25 contiguous amino acids of any one of SEQ ID NO:1-3. In some embodiments, the amino acid sequence of OAC comprises one or more amino acid substitutions as compared to any one of SEQ ID NO:1-3. In some embodiments, the amino acid sequence of olivetolic acid cyclase is based on SEQ ID NO:1-3 including one or more variant amino acids of the disclosure.

In some embodiments, the OAC of the engineered cell comprises one or more amino acid variations at position(s) selected from the group consisting of H5X¹, wherein X¹ is selected from the group consisting of G,A,C,P,V,L, I,M,F,Y,W,Q,E,K,R,S,T,Y,N,Q,D,E,K, and R; I7X², wherein X² is selected from the group consisting of G,A,C,P,V,L,M,FY,W,K,R,S,T,H,N,Q,D, and E; L9X³, wherein X³ is selected from the group consisting of G,A,C,P,V,I,M,F,Y,W,K,R,S,T, Y,H,N,Q,D,E,K,R; F23X⁴, wherein X⁴ is selected from the group consisting of G,A,C,P,V,L,I,M,Y,W,S,T,H,N,Q,D,E,K, and R; F24X⁵, wherein X⁵ is selected from the group consisting of G,A,C,P, V,I,M,Y,S,T,H,N,Q,D,E,K,R, and W; Y27X⁶, wherein X⁶ is selected from the group consisting of G,A,C,P,V,L,I,M,F,W,S,T,H,N, Q,D,E,K, and R; V59X⁷, wherein X⁷ is selected from the group consisting of G,A,C, P,L,I,M,F,Y,W,H,Q,E,K, and R; V61X⁸, wherein X⁸ is selected from the group consisting of G,A,C,P,L,I,M,F,Y,W,H,Q,E,K,R,S,T,N, and D; V66X⁹, wherein X⁹ is selected from the group consisting of G,A,C,P,L,I,M,F,Y, and W; E67X¹⁰, wherein X¹⁰ is selected from the group consisting of G,A,C,P,V,L,I,M,F,Y, and W; I69X¹¹, wherein X¹¹ is selected from the group consisting of G,A,C,P,V,L,M,F,Y, and W; Q70X¹², wherein X¹² is selected from the group consisting of S,T,H,N,D,E, R,K, and Y; 173X¹³, wherein X¹³ is selected from the group consisting of G,A,C,P, V,L,M,F,Y, and W; I74X¹⁴, wherein X¹⁴ is selected from the group consisting of G,A,C,P,V,L,M,F,Y, and W; V79X¹⁵, wherein X¹⁵ is selected from the group consisting of G,A,C,P,L,I,M,F,Y, and W; G80X¹⁶, wherein X¹⁶ is selected from the group consisting of A,C,P,V,L,I,M,F,Y,W,S,T,H,N,Q,D,E,K, and R; F81X⁷ wherein X¹⁷ is selected from the group consisting of G,A,C,P,V,L,I,M,Y,W,S,T,H, N,Q,D,E,R, and K; G82X¹⁸, wherein X¹⁸ is selected from the group consisting of A,C,P,V,L,I,M,F,Y,W,S,T,H,N,Q,E,K, and R; D83X¹⁹, wherein X¹⁹ is selected from the group consisting of S,T,H,Q,N,E, R,K, and Y; R86X²⁰, wherein X²⁰ is selected from the group consisting of S,T,H,Q, N,D,E,K, and Y; W89X², wherein X² is selected from the group consisting of G,A,C,P,V,L,I,M,F,Y,W,S,T,H,N,Q,D,E,K, and R; L92X²², wherein X²² is selected from the group consisting of G,A,C,P,V,I,M, F,Y, and W; I94X²³, wherein X² is selected from the group consisting of G,A,C,P, V,L,M,F,Y,W,K,R,S,T,Y,H,N,Q,D, and E; D96X²⁴, wherein X²⁴ is selected from the group consisting of S,T,H,Q,N,E,R,K, and Y; V46X²⁵, wherein X²⁵ is selected from the group consisting of G,A,C,P,L,I,M,F,Y, and W; T47X²⁶, wherein X²⁶ is selected from the group consisting of S,H,Q,N,D,E,R,K, and Y; Q48X²¹, wherein X²⁷ is selected from the group consisting of S,T,H,N,D,E,R,K, and Y; K49X²⁸, wherein X²⁸ is selected from the group consisting of S,T,H,Q,N, D,E,R, and Y; N50X²⁹, wherein X²⁹ is selected from the group consisting of G,A,C,P,V,L,I,M,F,Y, and W; and K51X³⁰, wherein X³⁰ is selected from the group consisting of S,T,H,Q,N,D,E,R, and Y, wherein the amino acid positions correspond to SEQ ID NO: 1, and wherein the non-natural OAC is not a single variant of K4A, H5A, H5L, H5Q, H5S, H5N, H5D, 17L, I7F, L9A, L9W, K12A, F23A, F23I, F23W, F23L, F24L, F24W, F24A, Y27F, Y27M, Y27W, V28F, V29M, K38A, V40F, D45A, H57A, V59M, V59A, V59F, Y72F, H75A, H78A, H78N, H78Q, H78S, H78D, or D96A.

In some embodiments, the OAC of the engineered cell comprises one or more amino acid variations at position(s) selected from the group consisting of H5X¹, wherein X¹ is selected from the group consisting of G,A,C,P,V,L,I,M,F,Y,W, Q,E,K,R,S,T,Y,N,Q,D,E,K, and R; I7X², wherein X² is selected from the group consisting of G,A,C,P,V,L,M,FY,W,K,R,S,T,H,N,Q,D, and E; L9X³, wherein X³ is selected from the group consisting of G,A,C,P,V,I,M,F,Y,W,K,R,S,T,Y,H,N,Q,D,E, K,R; F23X⁴, wherein X⁴ is selected from the group consisting of G,A,C,P,V,L,I,M, Y,W,S,T,H,N,Q,D,E,K, and R; F24X⁵, wherein X⁵ is selected from the group consisting of G,A,C,P,V,I,M,Y,S,T,H,N,Q,D,E,K,R, and W; Y27X⁶, wherein X⁶ is selected from the group consisting of G,A,C,P,V,L,I,M,F,W,S,T,H,N,Q,D,E,K, and R; V59X⁷, wherein X⁷ is selected from the group consisting of G,A,C,P,L,I,M,F,Y, W,H,Q,E,K, and R; V61X⁸, wherein X⁸ is selected from the group consisting of G,A,C,P,L,I,M,F,Y,W,H,Q,E,K,R,S,T,N, and D; V66X⁹, wherein X⁹ is selected from the group consisting of G,A,C,P,L,I,M,F,Y, and W; E67X¹⁰, wherein X¹⁰ is selected from the group consisting of G,A,C,P,V,L,I,M,F,Y, and W; I69X¹¹, wherein X¹¹ is selected from the group consisting of G,A,C,P,V,L,M,F,Y, and W; Q70X², wherein X² is selected from the group consisting of S,T,H,N,D,E,R,K, and Y; I73X¹³, wherein X¹³ is selected from the group consisting of G,A,C,P,V,L,M,F,Y, and W; I74X¹⁴, wherein X¹⁴ is selected from the group consisting of G,A,C,P,V,L, M,F,Y, and W; V79X¹⁵, wherein X¹⁵ is selected from the group consisting of G,A,C,P,L,I,M,F,Y, and W; G80X¹⁶, wherein X¹⁶ is selected from the group consisting of A,C,P,V,L,I,M,F,Y,W,S,T,H,N,Q,D,E,K, and R; F81X¹⁷, wherein X¹⁷ is selected from the group consisting of G,A,C,P,V,L,I,M,Y,W,S,T,H,N,Q,D,E,R, and K; G82X¹⁸, wherein X¹⁸ is selected from the group consisting of A,C,P,V,L,I,M, F,Y,W,S,T,H,N,Q,E,K, and R; D83X¹⁹, wherein X¹⁹ is selected from the group consisting of S,T,H,Q,N,E, R,K, and Y; R86X²⁰, wherein X²⁰ is selected from the group consisting of S,T,H,Q, N,D,E,K, and Y; W89X²¹, wherein X²¹ is selected from the group consisting of G,A,C,P,V,L,I,M,F,Y,W,S,T,H,N,Q,D,E,K, and R; L92X²²; wherein X²² is selected from the group consisting of G,A,C,P,V,I,M,F,Y, and W; I94X²³, wherein X² is selected from the group consisting of G,A,C,P,V,L, M,F,Y,W,K,R,S,T,Y,H,N,Q,D, and E; D96X²⁴, wherein X²⁴ is selected from the group consisting of S,T,H,Q,N,E,R,K, and Y; V46*X²⁵, wherein X²⁵ is selected from the group consisting of G,A,C,P,L,I,M,F,Y, and W; T47*X²⁶, wherein X²⁶ is selected from the group consisting of S,H,Q,N,D,E,R,K, and Y; Q48*X²⁷, wherein X² is selected from the group consisting of S,T,H,N,D,E,R,K, and Y; K49*X²⁸, wherein X is selected from the group consisting of S,T,H,Q,N, D,E,R, and Y; N50*X²⁹, wherein X²⁹ is selected from the group consisting of G,A,C,P,V,L,I,M, F,Y, and W; and K51*X³⁰, wherein X³⁰ is selected from the group consisting of S,T,H,Q,N,D,E,R, and Y, wherein the amino acid positions correspond to SEQ ID NO: 1, and wherein the non-natural OAC is not a single variant of K4A, H5A, H5L, H5Q, H5S, H5N, H5D, I7L, I7F, L9A, L9W, K12A, F23A, F23I, F23W, F23L, F24L, F24W, F24A, Y27F, Y27M, Y27W, V28F, V29M, K38A, V40F, D45A, H57A, V59M, V59A, V59F, Y72F, H75A, H78A, H78N, H78Q, H78S, H78D, or D96A, and wherein the “*” indicates amino acid residues from chain B of OAC dimer and corresponding to SEQ ID NO: 1.

In some embodiments, the engineered cell comprises one or more other enzymes in an olivetolic acid pathway, or in a cannabinoid pathway. In some embodiments, engineered cell has an olivetolic acid pathway comprising a variant OAC of the disclosure and an olivetol synthase.

In some embodiments, the OLS is a non-natural OLS having at least 60% identity to at least 25 or more contiguous amino acids of SEQ ID NO: 4. In some embodiments, the non-natural OLS comprises one or more amino acid substitutions at position(s) selected from the group consisting of: A125G, A125S, A125T, A125C, A125Y, A125H, A125N, A125Q, A125D, A125E, A125K, A125R, S126G, S126A, D185G, D185G, D185A, D185S, D185P, D185C, D185T, D185N, M187G, M187A, M187S, M187P, M187C, M187T, M187D, M187N, M187E, M187Q, M187H, M187H, M187V, M187L, M187I, M187K, M187R, L190G, L190A, L190S, L190P, L190C, L190T, L190D, L190N, L190E, L190Q, L190H, L190V, L190M, L190I, L190K, L190R, G204A, G204C, G204P, G204V, G204L, G204I, G204M, G204F, G204W, G204S, G204T, G204Y, G204H, G204N, G204Q, G204D, G204E, G204K, G204R, G209A, G209C, G209P, G209V, G209L, G209I, G209M, G209F, G209W, G209S, G209T, G209Y, G209H, G209N, G209Q, G209D, G209E, G209K, G209R, D210A, D210C, D210P, D210V, D210L, D2101, D210M, D210F, D210W, D210S, D210T, D210Y, D210H, D210N, D210Q, D210E, D210K, D210R, G211A, G211C, G211P, G211V, G211L, G2111, G211M, G211F, G211W, G211S, G211T, G211Y, G211H, G211N, G211Q, G211D, G211E, G211K, G211R, G249A, G249C, G249P, G249V, G249L, G2491, G249M, G249F, G249W, G249S, G249T, G249Y, G249H, G249N, G249Q, G249D, G249E, G249K, G249R, G249S, G249T, G249Y, G250A, G250C, G250P, G250V, G250L, G250I, G250M, G250F, G250W, G250S, G250T, G250Y, G250H, G250N, G250Q, G250D, G250E, G250K, G250R, L257V, L257M, L2571, L257K, L257R, L257F, L257Y, L257W, L257S, L257T, L257C, L257H, L257N, L257Q, L257D, L257E, F259G, F259A, F259C, F259P, F259V, F259L, F2591, F259M, F259Y, F259W, F259S, F259T, F259Y, F259H, F259N, F259Q, F259D, F259E, F259K, F259R, M331G, M331A, M331S, M331P, M331C, M331T, M331D, M331N, M331E, M331Q, M331H, M331V, M331L, M3311, M331K, M331R, S332G, and S332 Å corresponding to the amino acid positions of SEQ ID NO: 4.

In some embodiments, the engineered cell comprises a fusion of an OAC with all or portion of OLS. In some embodiments, the OAC protein and the OLS protein are part of the same polypeptide. In some embodiments, the all or portion of the OAC protein and all or portion of the OLS protein are part of the same polypeptide. In some embodiments, the OAC and/or OLS are non-natural proteins. In some embodiments, the OAC or a fragment thereof is fused with OLS or a fragment thereof. In some embodiments, the OAC protein is fused with the OLS protein through a linker molecule. In some embodiments, the N-terminus of the OAC protein or a fragment thereof is fused with the C-terminus of the OLS protein or its fragment. In some embodiments, the C-terminus of the OAC protein or fragment thereof is fused with the N-terminus of the OLS protein or its fragment.

In some embodiments, the engineered cell comprising a variant OAC of the disclosure further comprises enzymes for the geranyl pyrophosphate pathway. In some embodiments, the geranyl pyrophosphate pathway comprises geranyl pyrophosphate synthase. In some embodiments, the geranyl pyrophosphate pathway comprises a mevalonate (MVA) pathway, a non-mevalonate (MEP) pathway, an alternative non-MEP, non-MVA geranyl pyrophosphate pathway using isoprenol or prenol as a precursor, or a combination thereof, wherein the alternative non-MEP, non-MVA geranyl pyrophosphate pathway comprises one or more of the enzymes: alcohol kinase, alcohol diphosphate kinase, isopentenyl phosphate kinase, dimethylallyl phosphate kinase, isopentenyl diphosphate isomerase, and geranyl pyrophosphate synthase enzymes.

In some embodiments, the engineered cell comprises one or more exogenous nucleic acids, wherein at least one exogenous nucleic acid encodes the non-natural olivetolic acid cyclase. In some embodiments, the engineered cell comprises two or more exogenous nucleic acids, and wherein at least one exogenous nucleic acid encodes the non-natural OAS, and another exogenous nucleic acid encodes OLS. In some embodiments, the engineered cell comprises a nucleic acid encoding a polypeptide comprising all or portion of the OAC protein and all or portion of the OLS protein. In some embodiments, the OAC and/or OLS are non-natural proteins. In some embodiments, the N-terminus of the OAC protein or a fragment thereof is fused with the C-terminus of the OLS protein or its fragment. In some embodiments, the C-terminus of the OAC protein or fragment thereof is fused with the N-terminus of the OLS protein or its fragment. In some embodiments, the engineered cell comprises three or more exogenous nucleic acids, and wherein at least one exogenous nucleic acid encodes the non-natural OAS, an exogenous nucleic acid encodes OLS, and one exogenous nucleic acid encodes enzymes for producing geranyl pyrophosphate.

In some embodiments, the engineered cell is a prokaryote or a eukaryote. In some embodiments, the engineered cell is a eukaryote selected from the group consisting of yeast, fungi, microalgae, and algae. In some embodiments, the engineered cell is a prokaryote, e.g., Escherichia, Cyanobacteria, Corynebacterium, Bacillus, Ralstonia, Zymomonas, and Staphylococcus.

In embodiments, the engineered cell can produce olivetolic acid, or an analog or derivative thereof, or a cannabinoid, or an analog or derivative thereof, wherein the cell produces less olivetol, analogs or derivatives of olivetol, pentyl diacetic acid lactone (PDAL), hexanoyl triacetic acid lactone (HTAL), a lactone analog or derivatives thereof, or a combination thereof as compared to a wild-type non-engineered cell or an engineered cell comprising the wild-type OAC.

In embodiments, the olivetolic acid, cannabinoid, analog or derivative thereof can be present in a cell extract, or engineered cell culture medium, or a purified or refined preparation using the variant OAC of the disclosure. In some embodiments, the engineered cell, engineered cell extract, or engineered cell culture medium comprises olivetolic acid, analogs or derivatives thereof, or a combination thereof, at a concentration of 50% by weight or greater of the total products of non-natural OAC catalyzed reactions in combination with the activity of olivetolic acid cyclase. In some embodiments, the olivetol or its analogs, pentyl diacetic acid lactone (PDAL), hexanoyl triacetic acid lactone (HTAL), or lactone analog or derivatives thereof, or a combination thereof is present at a concentration of no more than about 50% to about 0.1% by weight of the cell extract or cell culture medium.

In another aspect, provided are method for forming an aromatic compound, comprising: (a) contacting an acyl-CoA and malonyl-CoA substrates with an olivetol synthase to form a polyketide, or analog or derivative thereof, (b) contacting the polyketides, or analog or derivative thereof with a non-natural olivetolic acid cyclase enzyme of the disclosure, wherein the contacting forms the aromatic compound. In some embodiments, the aromatic compound is olivetolic acid, analogs and derivatives thereof, or combinations thereof. In some embodiments, the method is carried out inside a cell. In some embodiments, the acyl-CoA substrate has a following structure:

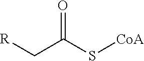

wherein R is a fatty acid side chain optionally comprising one or more functional and/or reactive groups as disclosed herein (i.e., an acyl-CoA compound derivative). In some embodiments, functional groups may include, but are not limited to, azido, halo (e.g., chloride, bromide, iodide, fluorine), methyl, alkyl (including branched and linear alkyl groups), alkynyl, alkenyl, methoxy, alkoxy, acetyl, amino, carboxyl, carbonyl, oxo, ester, hydroxyl, thio, cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, heterocyclyl, spirocyclyl, heterospirocyclyl, thioalkyl, sulfone, sulfonyl, sulfoxide, amido, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like.

In some embodiments, the reactive groups may include, but are not necessarily limited to, azide, carboxyl, carbonyl, amine, (e.g., alkyl amine (e.g., lower alkyl amine), aryl amine), halide, ester (e.g., alkyl ester (e.g., lower alkyl ester, benzyl ester), aryl ester, substituted aryl ester), cyano, thioester, thioether, sulfonyl halide, alcohol, thiol, succinimidyl ester, isothiocyanate, iodoacetamide, maleimide, hydrazine, alkynyl, alkenyl, and the like. A reactive group may facilitate covalent attachment of a molecule of interest. Functional and reactive groups may be optionally substituted with one or more additional functional or reactive groups.

In some embodiments, the acyl-CoA substrate is selected from the group consisting of acetyl-CoA, propionyl-CoA, butyryl-CoA, valeryl-CoA, hexanoyl-CoA, heptanoyl-CoA, octanoyl-CoA, nonanoyl-CoA, and decanoyl-CoA. In some embodiments, the non-natural OAC enzyme is present in a molar excess over the OLS enzyme. In some embodiments, the molar ratio of malonyl-CoA to acyl-CoA in the range of about 500:1 to about 1:500, about 250:1 to about 1:250, about 150:1, to about 10:1, to about 3:1, to about 1:150, about 100:1 to about 1:100, about 75:1 to about 1:75, about 50:1 to about 1:50, about 25:1 to about 1:25, about 15:1 to about 1:15, or about 10:1 to about 1:10.

In another aspect, provided are methods for forming a cannabinoid, an analog or derivatives thereof, comprising (a) contacting malonyl-CoA and an acyl-CoA substrates with a OLS that preferentially produces polyketides, analogs, and derivatives thereof, or combinations thereof over olivetol, analogs and derivatives of olivetol, pentyl diacetic acid lactone (PDAL), or lactone analogs and derivatives as compared to the wild type OLS; (b) contacting the polyketides, analogs and derivatives thereof, or combinations thereof with the non-natural OAC of this disclosure, wherein the contacting forms the olivetolic acid, analogs and derivatives thereof, or combinations thereof; (c) converting the olivetolic acid, or an analog or derivative thereof) to the cannabinoid, or an analog or derivative thereof, chemically or enzymatically, or by a combination of the both. In some embodiments, the aromatic compound is converted to the cannabinoid using a prenyltransferase. In some embodiments, the OLS is a non-natural OLS. In some embodiments, the method is carried out inside a cell. In some embodiments, the acyl-CoA substrate is selected from the group consisting of acetyl-CoA, propionyl-CoA, butyryl-CoA, valeryl-CoA, hexanoyl-CoA, heptanoyl-CoA, octanoyl-CoA, nonanoyl-CoA, and decanoyl-CoA. In some embodiments, the non-natural OAC enzyme is present in a molar excess over the OLS enzyme. In some embodiments, the molar ratio of malonyl-CoA to acyl-CoA in the range of about 500:1 to about 1:500, about 250:1 to about 1:250, about 150:1, to about 10:1, to about 3:1, to about 1:150, about 100:1 to about 1:100, about 75:1 to about 1:75, about 50:1 to about 1:50, about 25:1 to about 1:25, about 15:1 to about 1:15, or about 10:1 to about 1:10. In some embodiments, a cannabinoid derivative or cannabinoid precursor derivative produced by a genetically modified host cell disclosed herein or in a cell-free reaction mixture comprising one or more of the polypeptides disclosed herein. In some embodiments, a cannabinoid derivative or cannabinoid precursor derivative may comprise one or more chemical moieties. In some embodiments, the chemical moieties may include, but are not limited to, methyl, alkyl, alkenyl, methoxy, alkoxy, acetyl, carboxyl, carbonyl, oxo, ester, hydroxyl, aryl, heteroaryl, cycloalkyl, cycloalkenyl, cycloalkylalkenyl, cycloalkenylalkyl, cycloalkenylalkenyl, heterocyclylalkenyl, heteroarylalkenyl, arylalkenyl, heterocyclyl, aralkyl, cycloalkylalkyl, heterocyclylalkyl, heteroarylalkyl, and the like.

In another aspect, provided is a method for forming a 2,4-dihydroxy-6-alkylbenzoic acid from a 3,5,7-trioxoacyl-CoA or a 3,5,7-trioxocarboxylate substrate, wherein the 2,4-dihydroxy-6-alkylbenzoic acid is not olivetolic acid, and the 3,5,7-trioxoacyl-CoA or a 3,5,7-trioxocarboxylate substrate is not 3,5,7-trioxododecanoyl-CoA or 3,5,7-trioxododecanoate. The method comprises a) providing a 3,5,7-trioxoacyl-CoA or a 3,5,7-trioxocarboxylate substrate that is not 3,5,7-trioxododecanoyl-CoA or 3,5,7-trioxododecanoate, b) providing non-natural olivetolic acid cyclase comprising at least one amino acid variation as compared to a wild type OAC, wherein the non-natural OAC is enzymatically capable of: a) forming a 2,4-dihydroxy-6-alkylbenzoic acid from a 3,5,7-trioxoacyl-CoA or a 3,5,7-trioxocarboxylate substrate that is not 3,5,7-trioxododecanoyl-CoA or 3,5,7-trioxododecanoate at a greater rate as compared to the wild type OAC; (b) having a higher affinity for a 3,5,7-trioxoacyl-CoA or a 3,5,7-trioxocarboxylate substrate that is not 3,5,7-trioxododecanoyl-CoA or 3,5,7-trioxododecanoate as compared to the wild type OAC; or both a) and b), wherein the non-natural olivetolic acid cyclase is based on SEQ ID NO:1 or an OAC template having at least 60% identity to SEQ ID NO:1 or to at least 25 contiguous amino acids of SEQ ID NO:1, and the at least one amino acid variation is at position H5, I7, L9, F23, F24, Y27, V59, V61, V66, E67, I69, Q70, I73, I74, V79, G80, F81, G82, D83, R86, W89, L92, I94, D96, V46, T47, Q48, K49, N50, K51, V46*, T47*, Q48*, K49*, N50*, and K51*, wherein the “*” indicates residues from chain B of OAC dimer.

In some embodiments, the methods further include the step of isolating or purifying comprises one or more of continuous or non-continuous liquid-liquid extraction, pervaporation, evaporation, filtration, membrane filtration, reverse osmosis, nanofiltration, ultrafiltration, microfiltration, membrane filtration with diafiltration, membrane separation, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration.

In one aspect, provided are a composition comprising a cannabinoid, analogs, or derivatives thereof, or combinations thereof obtained from the engineered cell of the present disclosure, or the method of any of the present disclosure, wherein the composition comprises olivetol or analogs and derivatives of olivetol, pentyl diacetic acid lactone (PDAL), hexanoyl triacetic acid lactone (HTAL), a lactone analog, or a combination thereof at a concentration of no more than about 0.1% to about 0.0001% by weight of the composition.

In some embodiments, the composition is a cannabinoid, wherein the cannabinoid is cannabigerolic acid (CBGA), THCA, CBDA, CBCA, cannabigerol, THC, CBD, CBC, analogs or derivatives thereof, or a combination thereof. In some embodiments, the composition further comprises at least one pharmaceutically acceptable excipient selected from the group consisting of a diluent, a binder, a lubricant, a disintegrant, a flavoring agent, a coloring agent, a stabilizer, a surfactant, a glidant, a plasticizer, a preservative, an essential oil, a humectant, an absorption accelerator, a wetting agent, an absorber, and a buffering agent. In some embodiments, the composition is a pharmaceutical, an edible, personal care product, or a cosmetic.

### DESCRIPTION OF THE DRAWINGS

FIG. 1 shows an exemplary olivetolic acid synthesis pathway and exemplary cannabigerolic acid synthesis pathway. The terms tetraketide synthase (TKS) and olivetol synthase (OLS) are used interchangeably.

FIG. 2 shows the chemical structures of exemplary acyl-CoA substrate molecules that can be used in an olivetol synthase-catalyzed reaction.

FIG. 3 shows an alignment of SEQ ID NO: 1 (Cannabis sativa OAC) to another OAC homolog (SEQ ID NO:2) and to the stress-response A/B barrel domain-containing protein HS1 isoform X1 from Cicer arietinum (XP_004508017.1) SEQ ID NO:3.

FIG. 4 shows the exemplary pathway for producing olivetolic acid, analogs of olivetolic acid, cannabigerolic acid, analogs of cannabigerolic acid, cannabigerol and analogs of cannabigerol.

FIG. 5 shows the chemical structures of 3,5,7-trioxododecanoyl-CoA, PDAL, Olivetol, HTAL, olivetolic acid, and geranyl pyrophosphate.

FIG. 6 shows exemplary pathways of forming geranyl pyrophosphate from isoprenol.

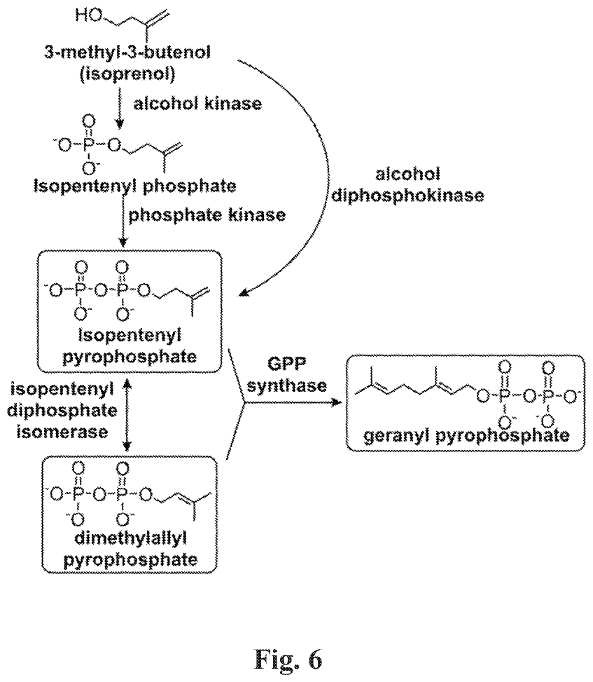

FIG. 7 shows exemplary pathways of forming geranyl pyrophosphate from prenol.

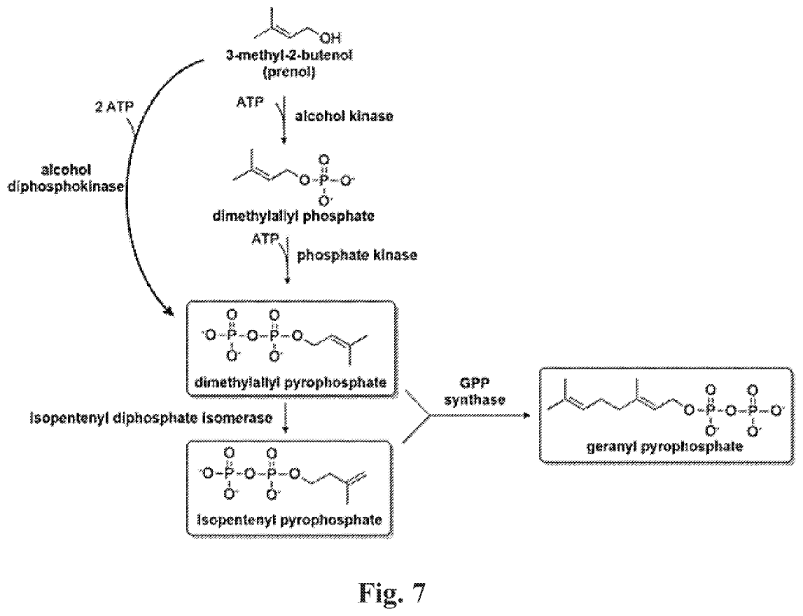

FIG. 8 shows exemplary mevalonate pathway (MVA) and non-mevalonate pathway (MEP). The abbreviations are DXS: 1-Deoxy-D-xylulose 5-phosphate synthase; DXR: 1-Deoxy-D-xylulose 5-phosphate reductoisomerase; CMS: 2-C-methyl-D-erythritol 4-phosphate cytidylyltransferase; CMK: 4-diphosphocytidyl-2-C-methyl-D-erythritol kinase; MECS: 2-C-methyl-D-erythritol 2,4-cyclodiphosphate synthase; HDS: 4-Hydroxy-3-methyl-but-2-enyl pyrophosphate synthase; HDR: 4-Hydroxy-3-methyl-but-2-enyl pyrophosphate reductase; DMAP: Dimethylallyl pyrophosphate; AACT: acetoacetyl-CoA thiolase; HMGS: HMG-CoA synthase; HMGR: HMG-CoA reductase; MVK: mevalonate-3-kinase; PMK: Phosphomevalonate kinase; MVD: mevalonate-5-pyrophosphate decarboxylase; and IDI: isopentenyl pyrophosphate isomerase.

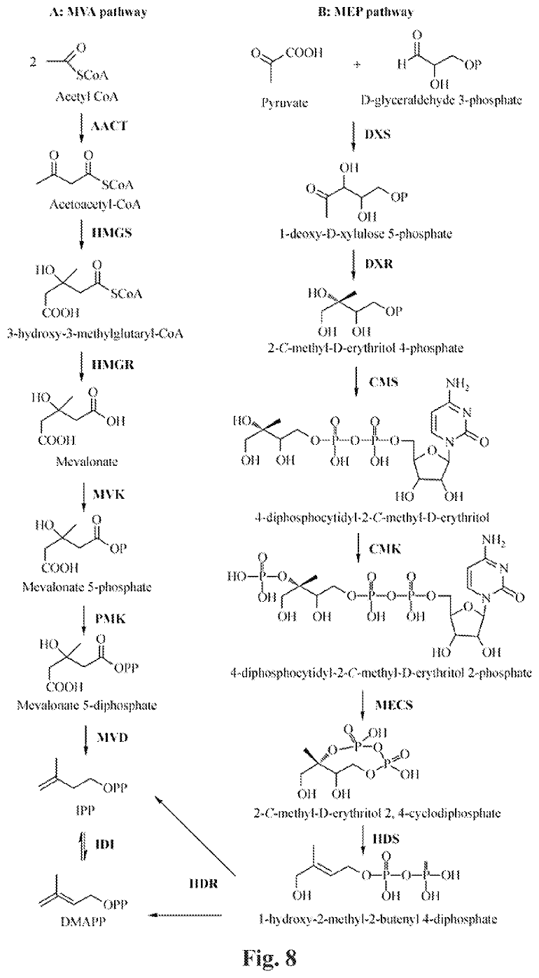

FIG. 9 shows the structures of olivetolic acid and exemplary analogs of olivetolic acid.

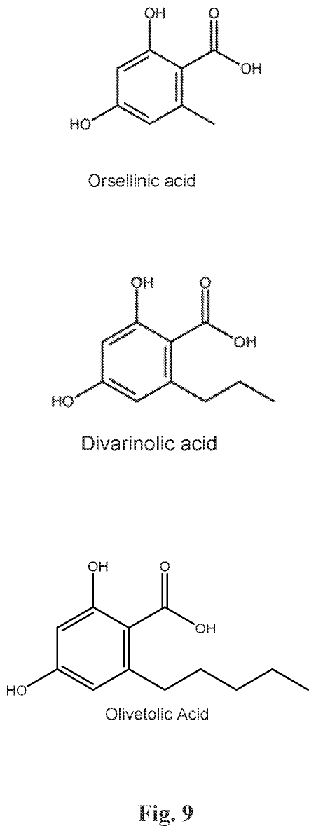

FIG. 10 shows the exemplary structures of 2,4-dihydroxy-6-alkylbenzoic acid, 3,5,7-trioxoacyl-CoA and 3,5,7-trioxocarboxylate in which the R group can be an acyl group with varying chain lengths, an aromatic group, a branched chain acyl group, substituted alkyl group (e.g., amino alkyl, hydroxyalkyl).

### DETAILED DESCRIPTION

The embodiments of the description described herein are not intended to be exhaustive or to limit the disclosure to the precise forms disclosed in the following detailed description. Rather, the embodiments are chosen and described so that others skilled in the art can appreciate and understand the principles and practices of the description.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Generally, the disclosure provides non-natural olivetolic acid cyclases (OACs) having at least one amino acid variation that differs from an amino acid residue of a wild type olivetolic acid cyclase. The non-natural OAC, in conjunction with a non-limiting amount of olivetol synthase, is enzymatically capable of: (a) forming a 2,4-dihydroxy-6-alkylbenzoic acid from a 3,5,7-trioxoacyl-CoA or a 3,5,7-trioxocarboxylate substrate; (b) forming a 2,4-dihydroxy-6-alkylbenzoic acid from a 3,5,7-trioxoacyl-CoA or a 3,5,7-trioxocarboxylate substrate at a greater rate as compared to the wild type OAC; (c) having a higher affinity for a 3,5,7-trioxoacyl-CoA or a 3,5,7-trioxocarboxylate substrate as compared to the wild type OAC; (d) with OLS, forming a 2,4-dihydroxy-6-alkylbenzoic acid from malonyl-CoA and acyl-CoA through a 3,5,7-trioxoacyl-CoA or a 3,5,7-trioxocarboxylate intermediate at a greater rate as compared to the wild type OAC, or any combination of (a), (b), (c) and (d). In some embodiments the non-natural OAC does not have a single mutation of Y27F with reference to SEQ ID NO:1; however, in other embodiments Y27F can be used in combination with one or more other amino acid variations as described herein.

As used herein the term “3,5,7-trioxoacyl-CoA substrate” or a “3,5,7-trioxocarboxylate substrate” refers to a substrate for OAC. In some embodiments, the OAC is a non-natural OAC. In some embodiments, the 3,5,7-trioxoacyl-CoA or the 3,5,7-trioxocarboxylate the substrate is converted to the 2,4-dihydroxy-6-alkylbenzoic acid product by the non-natural OAC of the disclosure. Exemplary structures of 3,5,7-trioxoacyl-CoA or a 3,5,7-trioxocarboxylate substrate and 2,4-dihydroxy-6-alkylbenzoic acid product is shown below:

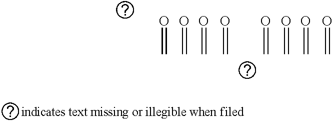

in which the R group can be an acyl group with varying chain lengths, an aromatic group, for example, benzoic, chorismic, phenylacetic and phenoxyacetic group, substituted alkyl group (e.g., amino alkyl, hydroxyalkyl) groups, branched chain acyl group. In some embodiments, non-limiting examples of amino alkyl group include aminoacyl 2-aminoacetyl, 3-aminopropionyl, 2-aminopropionyl, 4-aminobutyryl. In some embodiments, non-limiting examples of hydroxyalkyl group include 2-hydroxypropionyl, 3-hydroxybutyryl, hydroxyacetyl, hydroxypropionoyl, hydroxybutyryl. In some embodiments, branched chain acyl groups include isobutyryl or 3-methylbutyryl. In some embodiments, R is a fatty acid side chain optionally comprising one or more functional and/or reactive groups as disclosed herein (i.e., an acyl-CoA compound derivative). In some embodiments, functional groups may include, but are not limited to, azido, halo (e.g., chloride, bromide, iodide, fluorine), methyl, alkyl (including branched and linear alkyl groups), alkynyl, alkenyl, methoxy, alkoxy, acetyl, amino, carboxyl, carbonyl, oxo, ester, hydroxyl, thio, cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, heterocyclyl, spirocyclyl, heterospirocyclyl, thioalkyl, sulfone, sulfonyl, sulfoxide, amido, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like.

In some embodiments, the reactive groups may include, but are not necessarily limited to, azide, carboxyl, carbonyl, amine, (e.g., alkyl amine (e.g., lower alkyl amine), aryl amine), halide, ester (e.g., alkyl ester (e.g., lower alkyl ester, benzyl ester), aryl ester, substituted aryl ester), cyano, thioester, thioether, sulfonyl halide, alcohol, thiol, succinimidyl ester, isothiocyanate, iodoacetamide, maleimide, hydrazine, alkynyl, alkenyl, and the like. A reactive group may facilitate covalent attachment of a molecule of interest. Functional and reactive groups may be optionally substituted with one or more additional functional or reactive groups.

In some embodiments, 3,5,7-trioxoacyl-CoA or a 3,5,7-trioxocarboxylate substrate for OAC is formed from the OLS by condensation of a starter CoA molecule and malonyl-CoA.

In some embodiments, the starter CoA substrate has a following structure:

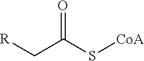

In some embodiments, R group can be an acyl group with varying chain lengths, an aromatic group, for example, benzoic, chorismic, phenylacetic and phenoxyacetic group, substituted alkyl group (e.g., amino alkyl, hydroxyalkyl) groups, branched chain acyl group. In some embodiments, non-limiting examples of amino alkyl group include aminoacyl 2-aminoacetyl, 3-aminopropionyl, 2-aminopropionyl, 4-aminobutyryl. In some embodiments, non-limiting examples of hydroxyalkyl group include 2-hydroxypropionyl, 3-hydroxybutyryl, hydroxyacetyl, hydroxypropionoyl, hydroxybutyryl. In some embodiments, branched chain acyl groups include isobutyryl, 3-methylbutyryl.

In some embodiments, R is a fatty acid side chain optionally comprising one or more functional and/or reactive groups as disclosed herein (i.e., an acyl-CoA compound derivative). In some embodiments, functional groups may include, but are not limited to, azido, halo (e.g., chloride, bromide, iodide, fluorine), methyl, alkyl (including branched and linear alkyl groups), alkynyl, alkenyl, methoxy, alkoxy, acetyl, amino, carboxyl, carbonyl, oxo, ester, hydroxyl, thio, cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, heterocyclyl, spirocyclyl, heterospirocyclyl, thioalkyl, sulfone, sulfonyl, sulfoxide, amido, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like.

In some embodiments, the reactive groups may include, but are not necessarily limited to, azide, carboxyl, carbonyl, amine, (e.g., alkyl amine (e.g., lower alkyl amine), aryl amine), halide, ester (e.g., alkyl ester (e.g., lower alkyl ester, benzyl ester), aryl ester, substituted aryl ester), cyano, thioester, thioether, sulfonyl halide, alcohol, thiol, succinimidyl ester, isothiocyanate, iodoacetamide, maleimide, hydrazine, alkynyl, alkenyl, and the like. A reactive group may facilitate covalent attachment of a molecule of interest. Functional and reactive groups may be optionally substituted with one or more additional functional or reactive groups.

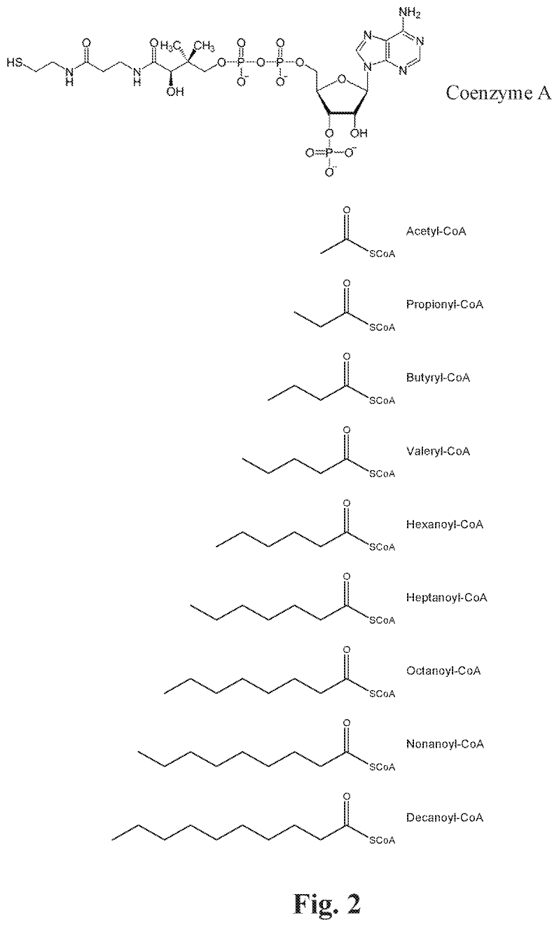

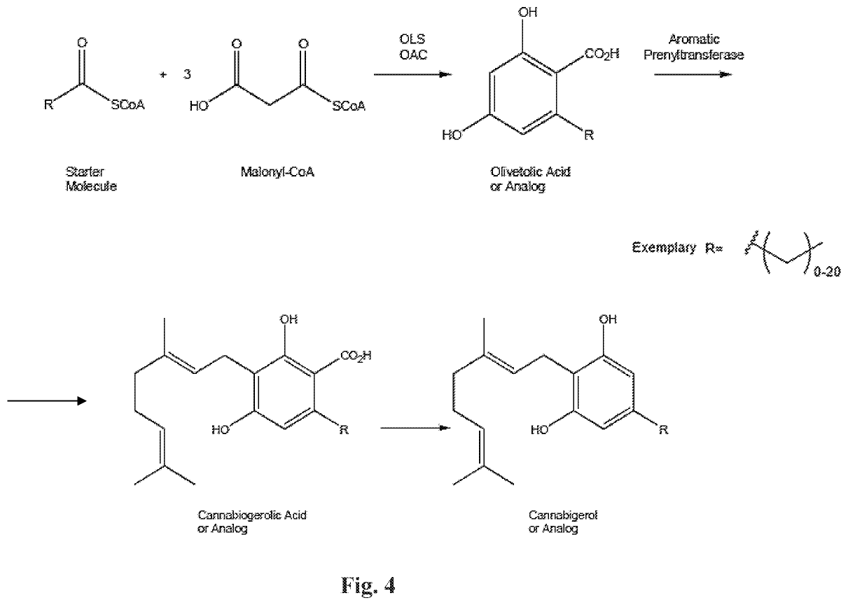

Exemplary starter CoA molecules include, but are not limited to, an aromatic acid CoA, for example, benzoic, chorismic, phenylacetic and phenoxyacetic acid CoA, acyl-CoA, aminoacyl-CoA (e.g., 2-aminoacetyl CoA, 3-aminopropionyl-CoA, 2-aminopropionyl-CoA, 4-aminobutyryl-CoA), hydroxyacyl-CoA (e.g., 2-hydroxypropionyl-CoA, 3-hydroxybutyryl-CoA, hydroxyacetyl-CoA, hydroxypropionoyl-CoA, hydroxybutyryl-CoA), branched chain acyl-CoA (e.g., isobutyryl-CoA, 3-methylbutyryl-CoA). Exemplary acyl-CoA include acetyl-CoA, propionyl-CoA, butyryl-CoA, valeryl-CoA, hexanoyl-CoA, heptanoyl-CoA, octanoyl-CoA, nonanoyl-CoA, decanoyl-CoA, one or more of C12, C14, C16, C18, C20 or C22 chain length fatty acid CoA. Exemplary acyl-CoA structures are shown in FIG. 2 and FIG. 4.

OAC from Cannabis sativa is a small protein of 12 kDa that is 101 amino acids in length. C. sativa OAC (UniProtKB Accession number 16WU39) is represented by SEQ ID NO:1 of the disclosure. C. sativa OAC produces olivetolic acid (OA) from 3,5,7-trioxododecanoyl-CoA. OAC, along with OLS, localizes to the cytoplasm using transient expression of fluorescent protein fusions in Nicotiana benthamiana leaves. Structurally, OAC is a dimeric α+β barrel (DABB) protein that is similar to DABB-type polyketide cyclase enzymes from Streptomyces and to stress-responsive proteins in plants (Gagne et al.). Olivetolic Acid Cyclase is classified under EC:4.4.1.26 under the Enzyme Commission nomenclature.

OAC from Cannabis sativa is a homodimeric protein, with each subunit consisting of same amino acid residues. The apo crystal structure of OAC was solved by the selenomethionine single-wavelength anomalous diffraction phasing of a selenomethionyl derivative (Se-SAD) method (Yang et al. FEBS J. 2016 March; 283(6):1088-106, which is incorporated by reference in its entirety). Significant conformational differences between monomers A and B were observed. The monomer A consists of a four-stranded antiparallel β-sheet and three α-helices (α1-α3), while the monomer B consists of a four-stranded antiparallel β-sheet and two α-helices. The outer surfaces of the antiparallel β-sheets face each other and form a central α+β barrel core.

Binary crystal structures of the OAC apo and OAC-OLA complex were solved showing the OAC protein has a unique active-site cavity containing the pentyl-binding hydrophobic pocket and the polyketide binding site according to Yang et al. (FEBS Journal 283:1088-1106; 2016). Site-directed mutagenesis studies indicate that the OAC amino acid residues Tyr72 and His78 function as acid/base catalysts at the catalytic center. Further, structural and/or functional studies of OAC suggested that the enzyme lacks thioesterase and aromatase activities.

In order to understand OAC structure and residues involved in substrate binding, molecular modeling was used to dock various linear tetraketide substrates into the OAC apo structure (SEQ ID NO:1). Random configurations of the ligand in the OAC active site were investigated and catalytically relevant configurations were identified. Residues within 5 Å of catalytically relevant and all other substrate binding conformations were identified. All residues within 5 Å of OLA were also within 5 Å of catalytically relevant substrate binding conformations.

Catalytically-relevant residues identified, and which can be subject to change to provide a variant amino acid in the non-natural OAC include positions H5, I7, L9, F23, F24, Y27, V59, V61, V66, E67, I69, Q70, Y72, I73, I74, H78, V79, G80, F81, G82, D83, R86, W89, L92, I94, D96, V46, T47, Q48, K49, N50, K51, V46*, T47*, Q48*, K49*, N50*, and K51*, wherein the “*” indicates residues from chain B of OAC dimer.

Residues near catalytically relevant substrate binding conformations are as follows H5, I7, L9, F23, F24, Y27, V59, V66, I69, Q70, I73, I74, V79, G80, F81, G82, D83, R86, W89, L92, I94, D96, V46, T47, Q48, K49, K51, V46*, T47*, Q48*, K49*, and K51*, and wherein the “*” indicates amino acid residues from chain B of OAC dimer and corresponding to SEQ ID NO: 1. Identified residues include the catalytic residues Y72 and His78.

In some embodiments one or more variant amino acid(s) in the non-natural OAC are at position(s) L9, F23, V59, V61, V66, E67, I69, Q70, I73, I74, V79, G80, F81, G82, D83, R86, W89, L92, I94, V46*, T47*, Q48*, K49*, N50*, and K51*, wherein the “*” indicates residues from chain B of OAC dimer.

In some embodiments, the non-natural OAC is not a single variant of K4A, H5A, K12A, K38A, D45A, H57A, H75A, H78A, or D96A of SEQ ID NO: 1.

In some embodiments, the non-natural OAC is not a single variant of H5L, H5Q, H5S, 17L, 17F, F24L, Y27F, Y27M, Y27W, V59M, Y72F, H78N, H78Q, or H78S of SEQ ID NO: 1.

In some embodiments, the non-natural OAC includes a single amino acid variation (mutation), wherein the variant amino acid has a side chain with similarities to the native (wild type) amino acid. Variants with similar side chains can be used to increase product formation by improving interaction of the linear tetraketide substrate with OAC. The interior of the active site binds the alkyl (e.g., pentyl) group of the substrate and product and is lined mostly with amino acids with hydrophobic side chains. Substitutions at these positions with other amino acids with hydrophobic side chains will result in altered binding of the alkyl group and thus higher 2,4-dihydroxy-6-alkylbenzoic acid (e.g., OLA) production. Residues outside and at the entrance to the active site are involved with binding of the ketone groups and CoA of the substrate. Substitutions at these positions with other amino acids with side chains with similar biochemical properties (hydrophobic, polar, charged, etc.) will result in altered binding of the substrate and thus higher 2,4-dihydroxy-6-alkylbenzoic acid (e.g., OLA) production. In some embodiments, the non-natural OAC includes a single amino acid variation (mutation) as shown in Table 1 and Table 6.

Accordingly, in embodiments, the non-natural OAC has one or more amino acid variations at position(s): H5X¹, wherein X¹ is selected from the group consisting of G,A,C,P,V,L,I,M,F,Y,W,Q,E,K,R,S,T,Y,N,Q,D,E,K, and R; 17X², wherein X² is selected from the group consisting of G,A,C,P,V,L,M,FY,W,K,R,S, T,H,N,Q,D, and E; L9X³, wherein X³ is selected from the group consisting of G,A, C,P,V,I,M,F,Y,W,K,R,S,T,Y,H,N,Q,D,E,K,R; F23X⁴, wherein X⁴ is selected from the group consisting of G,A,C,P,V,LI,M,Y,W,S,T,H,N,Q,D,E,K, and R; F24X⁵, wherein X⁵ is selected from the group consisting of G,A,C,P,V,I,M,Y,S,T,H,N,Q,D, E,K,R, and W; Y27X⁶, wherein X⁶ is selected from the group consisting of G,A,C,P, V,L,I,M,F,W,S,T,H,N,Q,D,E,K, and R; V59X⁷, wherein X⁷ is selected from the group consisting of G,A,C,P,L,I,M,F,Y,W,H,Q,E,K, and R; V61X⁸, wherein X⁸ is selected from the group consisting of G,A,C,P,L,I,M,F,Y,W,H,Q,E,K,R,S,T,N, and D; V66X⁹, wherein X⁹ is selected from the group consisting of G,A,C,P,L,I,M,F,Y, and W; E67X¹⁰, wherein X¹⁰ is selected from the group consisting of G,A,C,P,V,L,I, M,F,Y, and W; I69X¹¹, wherein X¹¹ is selected from the group consisting of G,A,C, P,V,L,M,F,Y, and W; Q70X¹², wherein X¹² is selected from the group consisting of S,T,H,N,D,E,R,K, and Y; 173X¹³, wherein X¹³ is selected from the group consisting of G,A,C,P,V,L,M,F,Y, and W; I74X¹⁴, wherein X¹⁴ is selected from the group consisting of G,A,C,P,V,L,M,F,Y, and W; V79X¹⁵, wherein X¹⁵ is selected from the group consisting of G,A,C,P,L,I,M,F,Y, and W; G80X¹⁶, wherein X¹⁶ is selected from the group consisting of A,C,P,V,L,I,M,F,Y,W,S,T,H,N,Q,D,E,K, and R; F81X¹⁷, wherein X¹⁷ is selected from the group consisting of G,A,C,P,V,L,I,M,Y,W, S,T,H,N,Q,D,E,R, and K; G82X¹⁸, wherein X¹⁸ is selected from the group consisting of A,C,P,V,L,I,M,F,Y,W,S,T,H,N,Q,E,K, and R; D83X¹⁹, wherein X¹⁹ is selected from the group consisting of S,T,H,Q,N,E, R,K, and Y; R86X²⁰, wherein X²⁰ is selected from the group consisting of S,T,H,Q,N,D,E,K, and Y; W89X², wherein X² is selected from the group consisting of G,A,C,P,V,L,I,M,F,Y,W,S,T,H,N,Q,D, E,K, and R; L92X², wherein X² is selected from the group consisting of G,A,C,P, V,I,M,F,Y, and W; I94X², wherein X²³ is selected from the group consisting of G,A,C,P,V,L,M,F,Y,W,K,R,S,T,Y,H,N,Q,D, and E; D96X²⁴, wherein X²⁴ is selected from the group consisting of S,T,H,Q,N,E,R,K, and Y; V46X²⁵, wherein X²⁵ is selected from the group consisting of G,A,C,P,L,I,M,F,Y, and W; T47X²⁶, wherein X²⁶ is selected from the group consisting of S,H,Q,N,D,E,R,K, and Y; Q48X²⁷ wherein X²⁷ is selected from the group consisting of S,T,H,N,D,E,K, and Y; K49X²⁸, wherein X²⁸ is selected from the group consisting of S,T,H,Q,N, D,E,R, and Y; N50X²⁹, wherein X²⁹ is selected from the group consisting of G,A,C,P,V,L, I,M,F,Y, and W; and K51X³⁰, wherein X³⁰ is selected from the group consisting of S,T,H,Q,N,D,E,R, and Y; V46*X³¹, wherein X³¹ is selected from the group consisting of G,A,C,P,L,I,M,F,Y, and W; T47*X³², wherein X³² is selected from the group consisting of S,H,Q,N,D,E,R,K, and Y; Q48*X³³, wherein X³³ is selected from the group consisting of S,T,H,N,D,E,R,K, and Y; K49*X³⁴, wherein X³⁴ s selected from the group consisting of S,T,H,Q,N, D,E,R, and Y; N50*X³⁵, wherein X³⁵ is selected from the group consisting of G,A,C;P,V,L,I,M,F,Y, and W; and K51*X³⁶, wherein X³⁶ is selected from the group consisting of S,T,H,Q,N,D,E,R, and Y, wherein the amino acid positions correspond to SEQ ID NO: 1, and wherein the non-natural OAC is not a single variant of K4A, H5A, H5L, H5Q, H5S, H5N, H5D, 17L, 17F, L9A, L9W, K12A, F23A, F23I, F23W, F23L, F24L, F24W, F24A, Y27F, Y27M, Y27W, V28F, V29M, K38A, V40F, D45A, H57A, V59M, V59A, V59F, Y72F, H75A, H78A, H78N, H78Q, H78S, H78D, or D96A, and wherein the “*” indicates amino acid residues from chain B of OAC dimer and corresponding to SEQ ID NO: 1.

In some embodiments, the non-natural OAC includes two, three, four, five, six, seven, eight, nine, ten, or more amino acid variation (mutation) as shown in Table 1 and Table 6.

TABLE 1

| Position | Mutation |
|---|---|
| H5 | G,A,C,P,V,L,I,M,F,Y,W |
| I7 | G,A,C,P,V,L,M,F,Y,W |
| L9 | G,A,C,P,V,I,M,F,Y,W |
| F23 | G,A,C,P,V,L,I,M,Y,W |
| F24 | G,A,C,P,V,L,I,M,Y,W |
| Y27 | G,A,C,P,V,L,I,M,F,W |
| V59 | G,A,C,P,L,I,M,F,Y,W |
| V61 | G,A,C,P,L,I,M,F,Y,W |
| V66 | G,A,C,P,L,I,M,F,Y,W |
| E67 | G,A,C,P,V,L,I,M,F,Y,W |
| I69 | G,A,C,P,V,L,M,F,Y,W |
| Q70 | S,T,H,N,D,E,R,K,Y |
| I73 | G,A,C,P,V,L,M,F,Y,W |
| I74 | G,A,C,P,V,L,M,F,Y,W |
| V79 | G,A,C,P,L,I,M,F,Y,W |
| G80 | A,C,P,V,L,I,M,F,Y,W |
| F81 | G,A,C,P,V,L,I,M,Y,W |
| G82 | A,C,P,V,L,I,M,F,Y,W |
| D83 | S,T,H,Q,N,E,R,K,Y |
| R86 | S,T,H,Q,N,D,E,K,Y |
| W89 | G,A,C,P,V,L,I,M,F,Y |
| L92 | G,A,C,P,V,I,M,F,Y,W |
| I94 | G,A,C,P,V,L,M,F,Y,W |
| D96 | S,T,H,Q,N,E,R,K,Y |
| V46* | G,A,C,P,L,I,M,F,Y,W |
| T47* | S,H,Q,N,D,E,R,K,Y |
| Q48* | S,T,H,N,D,E,R,K,Y |
| K49* | S,T,H,Q,N,D,E,R,Y |
| N50* | G,A,C,P,V,L,I,M,F,Y,W |
| K51* | S,T,H,Q,N,D,E,R,Y |

“*” indicates amino acid residues from chain B of OAC dimer and corresponding to SEQ ID NO: 1

In some embodiments, the non-natural OAC is enzymatically capable of: (a) forming 2,4-dihydroxy-6-alkylbenzoic acid from a 3,5,7-trioxoacyl-CoA or a 3,5,7-trioxocarboxylate substrate; (b) forming a 2,4-dihydroxy-6-alkylbenzoic acid from a 3,5,7-trioxoacyl-CoA or a 3,5,7-trioxocarboxylate substrate at a greater rate as compared to the wild type OAC; (c) having a higher affinity for a 3,5,7-trioxoacyl-CoA or a 3,5,7-trioxocarboxylate substrate as compared to the wild type OAC; (d) with OLS, forming a 2,4-dihydroxy-6-alkylbenzoic acid from malonyl-CoA and acyl-CoA through a 3,5,7-trioxoacyl-CoA or a 3,5,7-trioxocarboxylate intermediate at a greater rate as compared to the wild type OAC; (e) any combination of a), b), c), and d).

In some embodiments, the non-natural OAC includes one or more amino acid variation(s) designed to improve interaction of a 3,5,7-trioxoacyl-CoA or 3,5,7-trioxocarboxylate substrate that is different than 3,5,7-trioxododecanoyl-CoA or 3,5,7-trioxododecanoate, also referred to as “3,5,7-trioxododecanoyl-CoA analogs”.

In some embodiments, the 3,5,7-trioxododecanoyl-CoA analog includes a number of carbon atoms that is different than 3,5,7-trioxododecanoyl-CoA. In some embodiments, the 3,5,7-trioxododecanoyl-CoA analog can have a greater number of carbons, such as in the form of a longer alkyl group, as compared to 3,5,7-trioxododecanoyl-CoA. In some embodiments, 3,5,7-trioxoacyl-CoA or a 3,5,7-trioxocarboxylate substrates that are smaller and less hydrophobic than 3,5,7-trioxododecanoyl-CoA.

In some embodiments, a non-natural OAC can be designed to provide improved catalytic activity and/or affinity for 3,5,7-trioxoacyl-CoA or a 3,5,7-trioxocarboxylate substrates that are larger and more hydrophobic than 3,5,7-trioxododecanoyl-CoA. For example, when the substrate is a 3,5,7-3,5,7-trioxoacyl-CoAanalog that comprises a group longer than the pentyl group of 3,5,7-trioxododecanoyl-CoA, the non-natural OAC can have one or more relevant amino acid substitutions where the amino acid residues with bulky or large hydrophobic side chains are replaced with ones having smaller, less bulky hydrophobic side chains to accommodate the larger/bulkier substrate. Exemplary amino acid substitutions can be replacement of methionine, phenylalanine, or tryptophan to a small hydrophobic side chains such as glycine, alanine, valine, leucine, isoleucine, or proline.

Conversely, in some embodiments, a non-natural OAC can be designed to provide improved catalytic activity and/or affinity for 3,5,7-trioxoacyl-CoA or a 3,5,7-trioxocarboxylate substrates that are smaller and less hydrophobic than 3,5,7-trioxododecanoyl-CoA. As described herein, the 3,5,7-3,5,7-trioxoacyl-CoAanalog includes an alkyl group that is shorter than the pentyl group of 3,5,7-trioxododecanoyl-CoA, the non-natural OAC can have one or more relevant amino acid substitutions where the amino acid residues with smaller hydrophobic side chains are replaced with amino acids having larger, bulkier hydrophobic side chains. Exemplary amino acid substitutions can be replacement of glycine, alanine, valine, leucine, isoleucine, or proline to a large hydrophobic side chains such as methionine, phenylalanine, or tryptophan.

Yet other OAC variants can be designed to provide improved catalytic activity and/or affinity for 3,5,7-trioxoacyl-CoA or a 3,5,7-trioxocarboxylate substrates that have chemical changes such as those that introduce charge, increase charge, remove charge, or reduce charge. Corresponding changes in the non-natural OAC that can increase interaction of the modified substrate with the active site include those that introduce an opposite charge, increase an opposite charge, remove opposite charge, or reduce opposite charge. When a 3,5,7-trioxoacyl-CoA or a 3,5,7-trioxocarboxylate substrate having one or more polar or charged portion(s) is used, the non-natural OAC can be engineered to have amino acids with polar side chains such as serine, threonine, cysteine, tyrosine, histidine, glutamine, or asparagine or a charged side chains such as aspartic acid, glutamic acid, lysine, and arginine.

In turn, engineered cells including OAC variants of the disclosure can effectively utilize various 3,5,7-trioxoacyl-CoA or a 3,5,7-trioxocarboxylate substrates to form desired 2,4-dihydroxy-6-alkylbenzoic acids, which in turn can be used as substrates for forming different types of cannabinoid analogs and derivatives thereof.

Table 6 provides exemplary amino acids positions in the OAC, and the corresponding variant based on the nature of the substrate modification. In some embodiments, the non-natural OAC has one, two, three, four, five, six, seven, eight, nine, ten, or more amino acid variation(s) as shown in Table 6.

In some embodiments, the non-natural OAC has a higher affinity for a 3,5,7-trioxoacyl-CoA or a 3,5,7-trioxocarboxylate substrate that is larger and more hydrophobic than 3,5,7 trioxododecanoyl-CoA, and has one or more amino acid variations at position(s): H5X¹, wherein X¹ is selected from the group consisting of G,A,C,P,V; I7X², wherein X² is selected from the group consisting of G,A,C,P,V,L, and M; L9X³, wherein X³ is selected from the group consisting of G,A,C,P,V,I, and M; F23X⁴, wherein X⁴ is selected from the group consisting of G,A,C,P,V,L,I,M,Y, W,S,T,H,N,Q,D,E,K, and R; F24X⁵, wherein X⁵ is selected from the group consisting of G,A,C, P,V,L,I,M,Y,W,S,T,H,N,Q,D,E,K, and R; Y27X⁶, wherein X⁶ is selected from the group consisting of G,A,C,P,V,L,I,M,F,W,S,T,H,N,Q,D,E,K, and R; V59X⁷, wherein X⁷ is selected from the group consisting of G,A,C, and P; V61X⁸, wherein X⁸ is selected from the group consisting of G,A,C, and P; G80X¹⁶, wherein X¹⁶ is selected from the group consisting of A,C,P,V,L,I,M,F,Y,W,S,T,H,N, Q,D,E,K, and R; F81X¹⁷, wherein X¹⁷ is selected from the group consisting of Y and W; G82X¹⁸, wherein X¹⁸ is selected from the group consisting of A,C,P,V,L,I, M,F,Y,W,S,T,H,N,Q,D,E,K, and R; W89X²¹, wherein X²¹ is selected from the group consisting of G,A,C,P,V,L,I,M,F,Y,W,S,T,H,N,Q,D,E,K, and R; L92X², wherein X²² is selected from the group consisting of G,A,C,P,V,I, and M; and I94X²³, wherein X² is selected from the group consisting of G,A,C,P,V,L, and M.

In some embodiments, the non-natural OAC has a higher affinity for a 3,5,7-trioxoacyl-CoA or a 3,5,7-trioxocarboxylate substrate that is smaller and less hydrophobic than 3,5,7 trioxododecanoyl-CoA, and has one or more amino acid variations at position(s): H5X¹, wherein X¹ is selected from the group consisting of V,M,F,Y,W,Q,E, and K, and R; I7X², wherein X² is selected from the group consisting of L,M,F,Y,W,K, and R; L9X³, wherein X³ is selected from the group consisting of I,M,F,Y,W,K, and R; F23X⁴, wherein X⁴ is selected from the group consisting of Y and W; F24X⁵, wherein X⁵ is selected from the group consisting of Y and W; Y27X⁶, wherein X⁶ is selected from the group consisting of F and W; V59X⁷, wherein X⁷ is selected from the group consisting of M,F,Y,W,H,Q,E,K, and R; V61X⁸, wherein X⁸ is selected from the group consisting of M,F,Y,W,H,Q,E,K, and R; G80X¹⁶, wherein X¹⁶ is selected from the group consisting of A,C,P, and V; F81X¹⁷, wherein X¹⁷ is selected from the group consisting of G,A,C,P,V,L,I,M,Y,W, S,T,H,N,Q,D,E,K, and R; G82X¹⁸, wherein X¹⁸ is selected from the group consisting of A,C,P, and V; W89X²¹, wherein X²¹ is selected from the group consisting of F, and Y; L92X², wherein X² is selected from the group consisting of I,M,F,Y,W,K, and R; and I94X², wherein X² is selected from the group consisting of L,M,F,Y,W,K, and R.

In some embodiments, the non-natural OAC has a higher affinity for a 3,5,7-trioxoacyl-CoA or a 3,5,7-trioxocarboxylate substrate that is more polar and/or more charged than 3,5,7 trioxododecanoyl-CoA, and has one or more amino acid variations at position(s): H5X¹, wherein X¹ is selected from the group consisting of S,T,Y,N,Q,D,E,K, and R; I7X², wherein X² is selected from the group consisting of S,T,Y,H,N,Q,D,E,K, and R; L9X³, wherein X³ is selected from the group consisting of S,T,Y,H,N,Q,D,E,K, and R; F23X⁴, wherein X⁴ is selected from the group consisting of S,T,Y,H,N,Q,D,E,K, and R; F24X⁵, wherein X⁵ is selected from the group consisting of S,T,Y,H,N,Q,D,E,K, and R; Y27X⁶, wherein X⁶ is selected from the group consisting of S,T,H,N,Q,D,E,K, and R; V59X⁷, wherein X⁷ is selected from the group consisting of S,T,Y,H,N,Q,D,E,K, and R; V61X⁸, wherein X⁸ is selected from the group consisting of S,T,Y,H,N,Q,D,E,K, and R; G80X¹⁶, wherein X¹⁶ is selected from the group consisting of S,T,Y,H,N,Q,D,E,K, and R; F81X¹⁷, wherein X¹⁷ is selected from the group consisting of S,T,Y,H,N,Q,D,E,K, and R; G82X⁸, wherein X¹⁸ is selected from the group consisting of S,T,Y,H,N,Q,D,E,K, and R; W89X², wherein X² is selected from the group consisting of S,T,Y,H,N,Q,D,E,K, and R; L92X², wherein X²² is selected from the group consisting of S,T,Y,H,N,Q,D,E,K, and R; and I94X²³, wherein X²³ is selected from the group consisting of S,T,Y,H,N,Q,D,E,K, and R.

Optionally, the non-natural OAC variant that has a higher affinity for the 3,5,7-trioxododecanoyl-CoA analog also has a lower affinity for 3,5,7-trioxododecanoyl-CoA, as compared to the wild type OAC. Optionally, the non-natural OAC variant that has a higher rate of conversion of the 3,5,7-trioxododecanoyl-CoA analog also has a lower rate of conversion of 3,5,7-trioxododecanoyl-CoA, as compared to the wild type OAC.

In some embodiments, the amino acid sequence of the non-natural olivetolic acid cyclase is at least 60%, 65%, 70%, 75%, 80%, 85%, 90%, 95%, 99%, or 100% identical to at least 25, at least 30, at least 35, at least 40, at least 45, at least 50, at least 55, at least 60, at least 75, at least 80, at least 85, at least 90, or at least 95 contiguous amino acids of SEQ ID NO:1. In some embodiments the non-natural OAC include any one or more of the amino acid variations as set forth in Tables 1-3.

Although the positions recited herein are with reference to the corresponding amino acid sequence of SEQ ID NO:1, it is expressly contemplated that the amino acid sequence of a non-natural OAC that is different than SEQ ID NO:1 can have one or more amino acid variations at equivalent positions (variant positions) in the corresponding homologs of SEQ ID NO: 1. Identification of a template OAC can be based on best alignment of one or more template OAC(s) with SEQ ID NO:1. After alignment of SEQ ID NO:1 with one or more template OAC(s), identification of variant positions in can readily be understood.

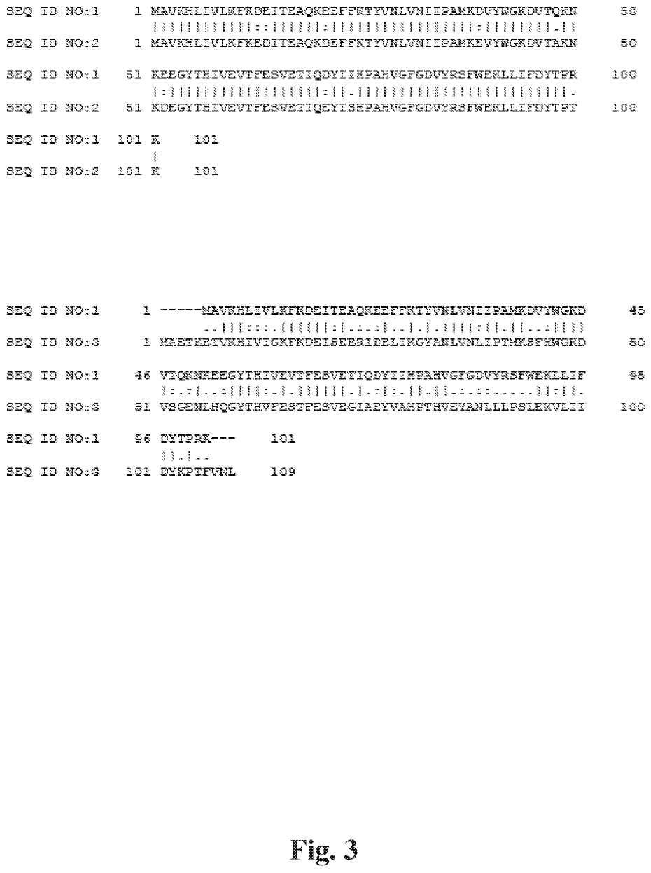

For example, FIG. 3 shows an alignment of SEQ ID NO:1 with the OAC homolog SEQ ID NO:2, which are both polypeptides of 101 amino acids and share a very high identity (91%). Variations as described herein for SEQ ID NO:1 can be made at the same amino acid position(s) for SEQ ID NO:2.

In some cases, alignment will show that a variant position is shifted a certain amount of amino acid positions from the variant position on SEQ ID NO:1. The shift can be reflected by an increase (e.g., “+x”) or a decrease.

For example, FIG. 3 shows an alignment of SEQ ID NO:1 with the stress-response A/B barrel domain-containing protein HS1 isoform X1 from Cicer arietinum (XP_004508017.1) SEQ ID NO:3, which share an identity of 52%. As shown in Figure X, there are five additional amino acids at the amino terminus of SEQ ID NO:3 prior to an alignment region between the two polypeptides. Accordingly, in SEQ ID NO:3 the variant positions are shifted +5, from these locations, and therefore SEQ ID NO:3 can have one or more amino acid variations at position(s) of: 10, 12, 14, 28, 29, 32, etc.

Further, other OACs that are different than SEQ ID NOs: 1-3 can be aligned to SEQ ID NO: 1 to identify variant positions and used to create non-natural OACs that are different than non-natural OACs based on SEQ ID NOs: 1-3 of the disclosure. In some embodiments, other OACs that are different than SEQ ID NOs 1-3, but having amino acid identity of 45% or greater, can be aligned to SEQ ID NO: 1 to identify corresponding variant amino acid positions and to make non-natural OACs based on information of the current disclosure.

In embodiments where the non-natural OAC is different than SEQ ID NO:1-3, the difference between those sequences and the SEQ ID NO:1-3 sequence can optionally be described with regards to “preferred invariable amino acid(s),” which are those amino acid location(s) that are preferably not substituted in a template that has less than 100% sequence identity to any one of SEQ ID NOs:1-3, with the exception of the particular variant or variant combinations described herein. Amino acids other that these preferred invariable amino acids can be substituted to provide for sequences having lower percentage identities than the template sequence. For example, in the non-natural OAC, some (50%, 60%, 70%, 80%, 85%, 90%, 93%, 95%, 97%, 98%, 99% or greater), or all (100%) of the following amino acids at the following locations do not vary from the referenced template at the following amino acid locations: 1M, 2A, 3V, 4K, 5H, 10K, 11F, 12K, 15I, 17E, 22E, 25K, 27Y, 29N, 30L, 31V, 32N, 34I, 35P, 37M, 38K, 42W, 43G, 44K, 45D, 46V, 50N, 54G, 55Y, 56T, 57H, 60E, 62T, 63F, 64E, 65S, 66V, 67E, 69I, 72Y, 75H, 76P, 78H, 79V, 90E, 91K, 93L, 94I, 96D, 97Y, and 99P; and more preferably, 1M, 2A, 3V, 4K, 5H, 6L, 7I, 8V, 9L, 10K, 11F, 12K, 13D, 14E, 15I, 16T, 17E, 18A, 19Q, 20K, 22E, 23F, 24F, 25K, 26T, 27Y, 28V, 29N, 30L, 31V, 32N, 33I, 34I, 35P, 36A, 37M, 38K, 40V, 41Y, 42W, 43G, 44K, 45D, 46V, 47T, 49K, 50N, 51K, 53E, 54G, 55Y, 56T, 57H, 58I, 59V, 60E, 61V, 62T, 63F, 64E, 65S, 66V, 67E, 68T, 69I, 70Q, 72Y, 73I, 75H, 76P, 77A, 78H, 79V, 80G, 81F, 82G, 83D, 84V, 85Y, 86R, 87S, 88F, 89W, 90E, 91K, 92L, 93L, 94I, 95F, 96D, 97Y, 98T, 99P, and 101K. With reference to SEQ ID NO:1, amino acid positions that can be varied include, but are not limited to, positions 39, 48, 52, 74, and 100.

For example, some of all of these invariable acids can be present in non-natural OACs having one or more amino acid variation(s) selected from the group consisting of H5, I7, L9, F23, F24, Y27, V59, V61, V66, E67, I69, Q70, I73, I74, V79, G80, F81, G82, D83, R86, W89, L92, I94, D96, V46, T47, Q48, K49, N50, and K51. For those amino acid positions, such as H5, where substitutions provide improved catalytic activity and/or affinity for the particular substrate, those substitutions when desired will control over the noted “invariable” amino acid at that position.

In some embodiments, the non-natural OAC with one or more variant amino acids as described herein, are enzymatically capable of forming a 2,4-dihydroxy-6-alkylbenzoic acid from a 3,5,7-trioxoacyl-CoA or a 3,5,7-trioxocarboxylate substrate at a rate of at least about 1.2, 1.5, 2, 3, 4, 5, 6, 7, 8, 9, 10, 12, 15, 20, or greater as compared to the wild type OAC or forming a 2,4-dihydroxy-6-alkylbenzoic acid.

In some embodiments, the non-natural OAC when used with a non-rate-limiting OLS, are enzymatically capable of forming a 2,4-dihydroxy-6-alkylbenzoic acid from malonyl-CoA and acyl-CoA through a 3,5,7-trioxoacyl-CoAintermediate at a rate of at least about 1.2, 1.5, 2, 3, 4, 5, 6, 7, 8, 9, 10, 12, 15, 20, or greater as compared to the wild type OAC.

In some embodiments, the OLS and OAC enzymes are present in equimolar amounts. In some embodiments, the amount of the non-natural OAC is present in a molar excess over OLS in an in vitro reaction or inside an engineered cell. In some embodiments, the amount of the OLS is present in a molar excess over the non-natural OAC in an in vitro reaction or inside an engineered cell. In some embodiments, the molar ratio of OAC to OLS is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or more. In some embodiments, the molar ratio of OLS to OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or more. In some embodiments, the OAC and/or the OLS is a non-natural enzyme.

In some embodiments, the rate of formation of olivetolic acid from 3,5,7,-3,5,7,trioxoacyl-CoA (non-limiting examples include 3,5,7-trioxododecanoyl-CoA, 3,5,7-trioxo-octanoyl-CoA, 3,5,7-trioxodecanoyl-CoA) or 3,5,7,-3,5,7-trioxocarboxylate (non-limiting examples include 3,5,7-trioxododecanoate, 3,5,7-trioxo-octanoate, 3,5,7-trioxodecanoate) by a non-natural OAC can be in the range of about 1.2 times to about 300 times, about 1.5 times to about 200 times, or about 2 times to about 30 times as compared to a wild-type OAC. In some embodiments, the rate of formation of olivetolic acid from 3,5,7,-3,5,7-trioxoacyl-CoA or 3,5,7,-3,5,7-trioxocarboxylate can be determined in an in vitro enzymatic reaction using a purified non-natural OAC. In some embodiments, the 3,5,7,-3,5,7-trioxoacyl-CoA or 3,5,7,-3,5,7-trioxocarboxylate is generated by OLS from acyl-CoA and malonyl-CoA.

In some embodiments, the total by-products (e.g., olivetol, analogs of olivetol, PDAL, HTAL, and other lactone analogs) of the olivetolic acid pathway using OLS and non-natural OAC, are in an amount (w/w) of less than about 50%, 45%, 40%, 35%, 30%, 25%, 20%, 15%, 12.5%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.025%, or 0.01% of the total weight of the products formed by OLS and OAC enzyme combinations. In some embodiments, the OLS can be a non-natural OLS.

Olivetol synthases are classified as EC:2.3.1.206 under the Enzyme Commission nomenclature. Olivetol synthases are homodimeric and have structural similarities with plant type III PKS enzymes. The OLS enzyme comprises conserved Cys157-His 297-Asn 330 catalytic triad, and the ‘gatekeeper’ Phe 208 corresponding to the amino acid positions of SEQ ID NO: 4. These amino acid residues are conserved for all other OLS homologs.

In some embodiments, olivetol synthase can catalyze the condensation of malonyl-CoA and starter CoA molecules to form polyketides. In some embodiments, the CoA molecules can be an acyl-CoA, aminoacyl-CoA (e.g., 2-aminoacetyl CoA, 3-aminopropionyl-CoA, 2-aminopropionyl-CoA, 4-aminobutyryl-CoA), hydroxyacyl-CoA (e.g., 2-hydroxypropionoyl-CoA, 3-hydroxybutyryl-CoA, hydroxyacetyl-CoA, hydroxypropionoyl-CoA, hydroxybutyryl-CoA), branched chain acyl-CoA (e.g., isobutyryl-CoA, 3-methylbutyryl-CoA), an aromatic acid CoA, for example, benzoic, chorismic, phenylacetic and phepoxyacetic acid CoA. Exemplary acyl-CoA include acetyl-CoA, propionyl-CoA, butyryl-CoA, valeryl-CoA, hexanoyl-CoA, heptanoyl-CoA, octanoyl-CoA, nonanoyl-CoA, decanoyl-CoA, one or more of C12, C14, C16, C18, C20 or C22 chain length fatty acid CoA. Chemical formulas for exemplary starter CoA molecules are shown in FIGS. 2 and 4.

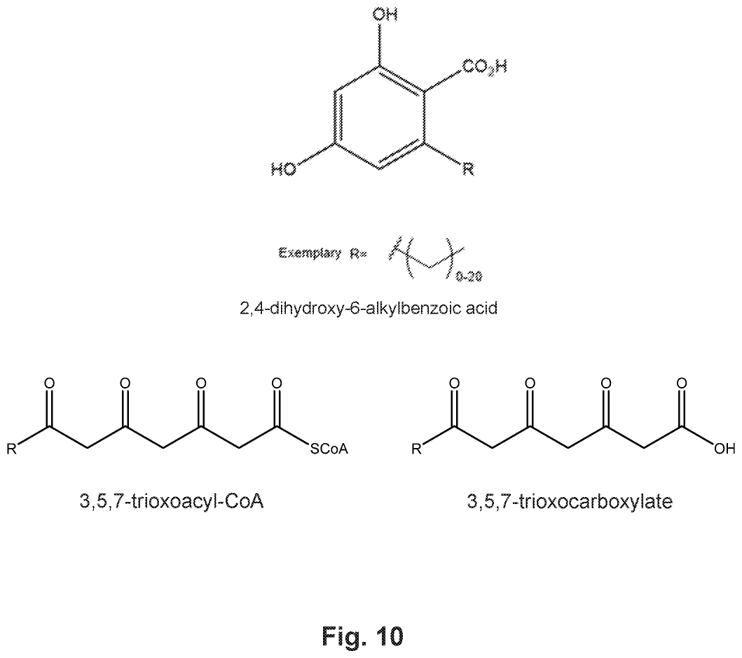

Based on the starter CoA molecule, the polyketides formed by OLS will differ. Exemplary polyketides are shown in FIG. 10. The polyketides are converted to olivetolic acid and its analogs by the OAC. In some embodiments, the OAC is non-natural olivetolic acid cyclase enzyme of the disclosure and the polyketides are 3,5,7 trioxoacyl-CoA. The table below shows exemplary products of OAC and OLS.

TABLE 2
Exemplary OAC and OLS products

| | | |
|---|---|---|
| Starting molecules | OLS Product (trioxoacyl- | OAC Product |
| | CoA) | |
| Hexanoyl-CoA, malonyl- | 3,5,7-trioxododecanoyl-CoA | Olivetolic Acid |
| CoA | | |
| Acetyl-CoA, malonyl-CoA | 3,5,7-trioxo-octanoyl-CoA | Orsellinic acid |
| Butyryl-CoA, malonyl-CoA | 3,5,7-trioxodecanoyl-CoA | Diyarinolic acid |

In the absence of OAC, the polyketides are otherwise hydrolyzed to lactones, e.g., pentyl diacetic acid lactone (PDAL), hexanoyl triacetic acid lactone (HTAL), or other lactone analogs depending on the starting substrates. Tetraketide and triketide pyrones were reported to be the reaction products of various type III PKSs, and triketide pyrone could be a derailment product from a premature intermediate.

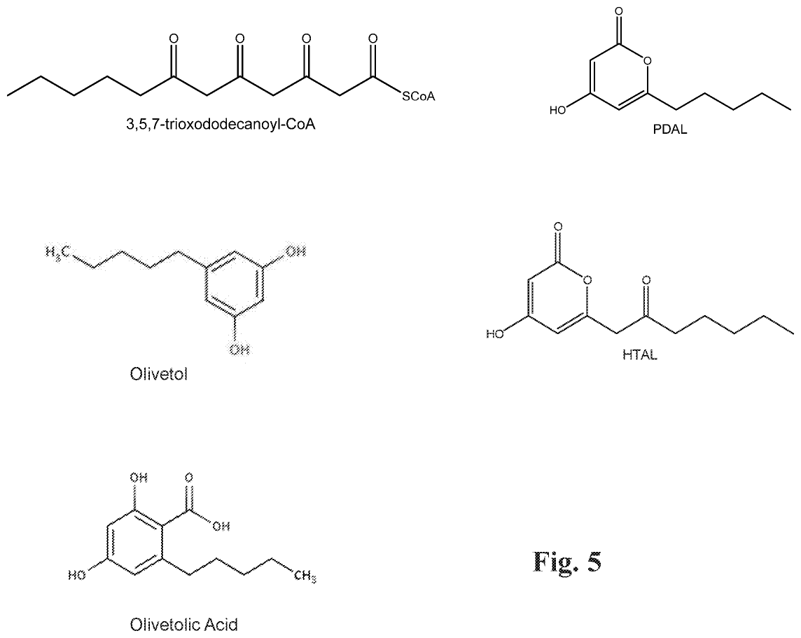

An exemplary polyketide generated by OLS is 3,5,7-trioxododecanoyl-CoA. Exemplary byproducts of the olivetolic acid pathway are olivetol, PDAL, HTAL, or its analogs or derivatives. Olivetol has the chemical names 5-pentylbenzene-1,3-diol, 5-pentylresorcinol, and 5-pentyl-1,3-benzenediol. PDAL, a by-product of olivetol synthase-catalyzed reaction, has the chemical name pentyl diacetic acid lactone. HTAL, another by-product of OLS-catalyzed reaction has the chemical name hexanoyl triacetic acid lactone. The chemical structures of 3,5,7-trioxododecanoyl-CoA, olivetol, olivetolic acid (2,4-dihydroxy-6-pentylbenzoic acid PDAL, and HTAL are shown in FIG. 5.

In some embodiments, the OLS can be a non-natural OLS. In some embodiments, the engineered cell comprises a non-natural OLS in addition to a non-natural OAC. In some embodiments, the non-natural OLS has at least about 50%, 60%, 65%, 70%, 75%, 80%, 85%, 87.5%, 90%, 92.5%, 95%, 99% or 100% sequence identity to at least 10, 25, 30, 35, 40, 50, 55, 60, 70, 75, 80, 90, 95, 100, 110, 120, 130, 140, 150, 160, 170, 180, 190, 200, 250, 300, 350 or more, or all, contiguous amino acids of SEQ ID NO:4. In some embodiments, the amino acid sequence of the non-natural OLS has one or more amino acid variations at position(s) selected from the group consisting of: 125, 126, 185, 187, 190, 204, 209, 210, 211, 249, 250, 257, 259, 331, and 332 corresponding to the amino acid sequence of SEQ ID NO:4.

In some embodiments, the amino acid substitutions designed to increase olivetolic acid production by OLS are shown below in Table 3. The amino acid positions of OLS corresponds to SEQ ID NO: 4. It is expressly contemplated that the amino acid sequence of the non-natural olivetol synthase can have one or more amino acid variations at equivalent positions corresponding to the homologs of SEQ ID NO: 4.

TABLE 3

| Position | Substitution |
|---|---|
| A125 | G,S,T,C,Y,H,N,Q,D,E,K,R |
| S126 | G,A |
| D185 | G,A,S,P,C,T,N |
| M187 | G,A,S,P,C,T,D,N,E,Q,H,V,L,I,K,R |
| L190 | G,A,S,P,C,T,D,N,E,Q,H,V,M,I,K,R |
| G204 | A,C,P,V,L,I,M,F,W |
| G209 | A,C,P,V |
| D210 | A,C,P,V |
| G211 | A,C,P,V |
| G249 | A,C,P,V,L,I,M,F,W,S,T,Y,H,N,Q,D,E,K,R |
| G250 | A,C,P,V,L,I,M,F,W,S,T,Y,H,N,Q,D,E,K,R |
| L257 | V,M,I,K,R,F,Y,W,S,T,C,H,N,Q,D,E |
| F259 | G,A,C,P,V,L,I,M,Y,W,S,T,Y,H,N,Q,D,E,K,R |
| M331 | G,A,S,P,C,T,D,N,E,Q,H,V,L,I,K,R |
| S332 | G,A |

In some embodiments the engineered cell includes a non-natural OAC as described herein and a non-natural OLS (either where the OAC polypeptide is independent of the OLS polypeptide, or where OAC and OLS are fused together) that includes one or more amino acid substitutions at position(s) selected from the group consisting of: Q82S, P131A, I186F, M187E, M187N, M187T, M187I, M187S, M187A, M187L, M187G, M187V, M187C, S195K, S195M, S195R, S197G, S197V, T239E, K314D, and K314M, corresponding to the amino acid positions of SEQ ID NO:4.

In embodiments non-natural olivetol synthase comprises two, or more than two amino acid substitutions, selected from: (i) Q82S and P131A, (ii) Q82S and M187S, (iii) Q82S and S195K, (iv) Q82S and S195M, (v) Q82S and S197V, (vi) Q82S and K314D, (vii) P131A and I186F, (viii) P131A and M187S, (ix) P131A and S195M, (x) P131A and S197V, (xi) P131A and K314D, (xii) P131A and K314M, (xiii) I186F and M187S, (xiv) I186F and S195K, (xv) I186F and S195M, (xvi) I186F and T239E, (xvii) I186F and K314D, (xviii) M187S and S195K, (xix) M187S and S195M, (xx) M187S and S197V, (xxi) M187S and T239E, (xxii) M187S and K314D, (xxiii) M187S and K314M, (xxiv) S195K and S197V, (xxv) S195M and S197V, (xxvi) S195M and T239E, (xxvii) S195K and K314D, (xxviii) S195K and K314M, (xxix) S195M and K314D, (xxx) S195M and K314M, (xxxi) S197V and T239E, (xxxii) S197V and K314M, (xxxiii) T239E and K314D, (xxxiv) T239E and K314M, (xxxv) Q82S and I186F, (xxxvi) Q82S and T239E, (xxxvii) Q82S and K314M, (xxxviii) I186F and S197V (xxxix) I186F and K314M, (xl) S195K and T239E, (xli) S197V and K314D, (xlii) P131A and T239E, and (xliii) P131A and S195K. The two or more of the recited substitutions of any of (i) to (xliii) can be made in SEQ ID NO:4, an olivetol synthase having sequence identity to SEQ ID NO:1 (e.g., at least about 50%, 75%, 90%, 93%, 94%, 95%, 96%, 97%, 98%, 99% identity, etc.),

In embodiments non-natural olivetol synthase comprises three, or more than three, amino acid substitutions selected from: (i) Q82S, P131A, and I186F, (ii) Q82S, P131A, and M187S, (iii) Q82S, P131A, and S195K, (iv) Q82S, P131A, and S195M, (v) Q82S, P131A, and S197V, (vi) Q82S, P131A, and T239E, (vii) Q82S, P131A, and K314D, (viii) Q82S, P131A, and K314M, (ix) Q82S, I186F, and M187S, (x) Q82S, I186F, and S195M, (xi) Q82S, I186F, and S197V, (xii) Q82S, I186F, and T239E, (xiii) Q82S, I186F, and K314D, (xiv) Q82S, I186F, and K314M, (xv) Q82S, M187S, and S195K, (xvi) Q82S, M187S, and S195M, (xvii) Q82S, M187S, and S197V, (xviii) Q82S, M187S, and T239E, (xix) Q82S, M187S, and K314D, (xx) Q82S, M187S, and K314M, (xxi) Q82S, S195K, and S197V, (xxii) Q82S, S195M, and S197V, (xxiii) Q82S, S195K, and K314D, (xxiv) Q82S, S195K, and K314M, (xxv) Q82S, S195M, and K314D, (xxvi) Q82S, S195M, and K314M, (xxvii) Q82S, S197V, and T239E, (xxviii) Q82S, S197V, and K314D, (xxix) Q82S, S197V, and K314M, (xxx) Q82S, T239E, and K314D, (xxxi) Q82S, T239E, and K314M, (xxxii) P131A, I186F, and M187S, (xxxiii) P131A, I186F, and S195K, (xxxiv) P131A, I186F, and S195M, (xxxv) P131A, I186F, and S197V, (xxxvi) P131A, I186F, and K314D, (xxxvii) P131A, I186F, and K314M, (xxxviii) P131A, M187S, and S195K, (xxxix) P131A, M187S, and S195M, (xl) P131A, M187S, and S197V, (xli) P131A, M187S, and T239E, (xlii) P131A, M187S, and K314D, (xliii) P131A, S195M, and S197V, (xliv) P131A, S195M, and T239E, (xlv) P131A, S195K, and K314D, (xlvi) P131A, S195K, and K314M, (xlvii) P131A, S195M, and K314D, (xlviii) P131A, S195M, and K314M, (xlix) P131A, S197V, and T239E, (1) P131A, S197V, and K314D, (li) P131A, S197V, and K314M, (lii) P131A, T239E, and K314D, (liii) P131A, T239E, and K314M, (liv) I186F, M187S, and S195K, (lv) I186F, M187S, and S195M, (lvi) I186F, M187S, and S197V, (lvii) I186F, M187S, and K314M, (lviii) I186F, S195K, and S197V, (lix) I186F, S195M, and S197V, (lx) I186F, S195K, and T239E, (lxi) I186F, S195M, and T239E, (lxii) I186F, S195K, and K314D, (lxiii) I186F, S195K, and K314M, (lxiv) I186F, S195M, and K314D, (lxv) I186F, S195M, and K314M, (lxvi) I186F, S197V, and T239E, (lxvii) I186F, S197V, and K314D, (lxviii) I186F, S197V, and K314M, (lxix) I186F, T239E, and K314M, (lxx) M187S, S195K, and S197V, (lxxi) M187S, S195M, and S197V, (lxxii) M187S, S195K, and T239E, (lxxiii) M187S, S195M, and T239E, (lxxiv) M187S, S195K, and K314D, (lxxv) M187S, S195K, and K314M, (lxxvi) M187S, S195M, and K314D, (lxxvii) M187S, S195M, and K314M, (lxxviii) M187S, S197V, and T239E, (lxxix) M187S, S197V, and K314D, (lxxx) M187S, S197V, and K314M, (lxxxi) M187S, T239E, and K314D, (lxxxii) M187S, T239E, and K314M, (lxxxiii) S195K, S197V, and T239E, (lxxxiv) S195M, S197V, and T239E, (lxxxv) S195K, S197V, and K314D, (lxxxvi) S195K, S197V, and K314M, (lxxxvii) S195M, S197V, and K314D, (lxxxviii) S195M, S197V, and K314M, (lxxxix) S195K, T239E, and K314D, (xc) S195K, T239E, and K314M, (xci) S195M, T239E, and K314D, (xcii) S195M, T239E, and K314M, and (xciii) S197V, T239E, and K314M. The three or more of the recited substitutions of any of (i) to (xciii) can be made in SEQ ID NO:4, or an olivetol synthase having sequence identity to SEQ ID NO:4.

In some embodiments, the non-natural OLS with one or more variant amino acids as described herein are enzymatically capable of preferentially forming polyketides as opposed to PDAL, HTAL, or other lactone analogs as compared to the wild-type enzyme. The polyketides can be hydrolyzed to PDAL, HTAL, and other lactone analogs depending on the starting substrates, or the polyketides can be converted to olivetol and its analogs by olivetol synthase. The polyketides also can be substrates for the non-natural OAC of the disclosure, which converts the polyketides to olivetolic acid and its analogs depending on the starting substrates.

In some embodiments, non-natural olivetol synthase with one or more variant amino acids as described herein are enzymatically capable of at least about 1.2, 1.5, 2, 3, 4, 5, 6, 7, 8, 9, 10, 12, 15, 20, or greater rate of formation of olivetolic acid and/or olivetol from malonyl-CoA and hexanoyl-CoA in the presence of a non-rate limiting amount of non-natural OAC enzyme, as compared to the wild type olivetol synthase. For example, in the presence of a non-rate limiting amount of non-natural OAC, the increase in rate of formation of olivetolic acid from malonyl-CoA and hexanoyl-CoA, as compared to the wild olivetol synthase, can be in the range of about 1.2 times to about 300 times, about 1.5 times to about 200 times, or about 2 times to about 30 times as determined in an in vitro enzymatic reaction using purified olivetol synthase variant.

In some embodiments, the total by-products (e.g., olivetol, analogs of olivetol, PDAL, HTAL, and other lactone analogs) of the non-natural olivetol synthase reaction products in the presence of molar excess of OAC, are in an amount (w/w) of less than about 50%, 45%, 40%, 35%, 30%, 25%, 20%, 15%, 12.5%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.025%, or 0.01% of the total weight of the products formed by OLS and OAC enzyme combinations.

In some embodiments, in addition to the non-natural OAC, the engineered cell also includes a non-natural OLS with one or more amino acid substitutions designed to alter the starter molecule specificity of the OLS enzyme. When the non-natural OAC of the disclosure is designed to improve interaction of a 3,5,7-trioxoacyl-CoA or a 3,5,7-trioxocarboxylate substrate that is different than 3,5,7-trioxododecanoyl-CoA, a corresponding variation can be made in the non-natural OLS to increase interaction of substrates that are used to form the OAC substrate (OLS product).

As described herein, when the non-natural OAC is designed to provide improved catalytic activity and/or affinity for 3,5,7-trioxoacyl-CoA or a 3,5,7-trioxocarboxylate substrates that are larger and more hydrophobic than 3,5,7-trioxododecanoyl-CoA, an OLS variant can be designed to provide improved catalytic activity and/or affinity for an acyl-CoA substrate that is larger than hexanoyl CoA (e.g., heptanoyl-CoA, octanoyl-CoA, nonanoyl-CoA, decanoyl-CoA). When the non-natural OAC is designed to provide improved catalytic activity and/or affinity for 3,5,7-trioxoacyl-CoA or a 3,5,7-trioxocarboxylate substrates that are smaller and less hydrophobic than 3,5,7-trioxododecanoyl-CoA, an OLS variant can be designed to provide improved catalytic activity and/or affinity for an acyl-CoA substrate that is smaller than hexanoyl CoA (e.g., acetyl-CoA, propionyl-CoA, butyryl-CoA, valeryl-CoA).

Table 4 provides exemplary amino acids positions in the non-natural OLS, and the corresponding variant based on the nature of the substrate modification. In some embodiments, the non-natural OLS has one, two, three, four, five, six, seven, eight, nine, or ten amino acid variation(s) as shown in Table 5.

TABLE 4

| | Analogs with | Analogs with smaller, | Analogs with |
|---|---|---|---|
| | larger, | hydrophobic starter | polar or charged |
| | hydrophobic | | |
| Position | starter molecules | molecules | starter molecules |
| G204 | A,C,P,V | A,C,P,V, L,I,M,F,W | S,T,Y,H,N,Q,D,E,K,R |
| G209 | A,C,P,V | A,C,P,V,L,I,M,F,W | S,T,Y,H,N,Q,D,E,K,R |
| D210 | A,C,P,V | A,C,P,V,L,I,M,F,W | S,T,Y,H,N,Q,E,K,R |
| G211 | A,C,P,V | A,C,P,V,L,I,M,F,W | S,T,Y,H,N,Q,D,E,K,R |
| G249 | A,C,P,V | A,C,P,V,L,I,M,F,W | S,T,Y,H,N,Q,D,E,K,R |
| G250 | A,C,P,V | A,C,P,V,L,I,M,F,W | S,T,Y,H,N,Q,D,E,K,R |
| F259 | G,A,C,P,V,L, | M,Y,W | S,T,Y,H,N,Q,D,E,K,R |
| | I,M,Y,W,S, | | |
| | T, H,N,Q,D,E,K,R | | |

OLS and OLS variants are described in commonly-assigned International Application No. PCT/US2020/028766, filed Apr. 17, 2020 (Noble et al.; Ref. No. GNO0107/WO).

As used herein the term “non-naturally occurring”, when used in reference to an organism (e.g., microbial) is intended to mean that the organism has at least one genetic alteration not normally found in a naturally occurring organism of the referenced species. Naturally-occurring organisms can be referred to as “wild-type” such as wild type strains of the referenced species.

As used herein the term “non-naturally occurring” and “variant” and “mutant” are used interchangeably in the context of a polypeptide or nucleic acid. The term “non-naturally occurring” and “variant” “mutant” in this context refers to a polypeptide or nucleic acid sequence having at least one variation/mutation at an amino acid position or a nucleic acid position as compared to a wild-type sequence.

Naturally-occurring organisms, nucleic acids, and polypeptides can be referred to as “wild-type” or “original” or “natural” such as wild type strains of the referenced species. Likewise, amino acids found in polypeptides of the wild type organism can be referred to as “original” or “natural” with regards to any amino acid position.

A genetic alteration that makes an organism non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

For example, in order to provide an OAC variant, C. sativa OAC (Accession number 16WU39) is represented by SEQ ID NO:1 of the disclosure., can be selected as a template. Variants, as described herein, can be created by introducing into the template one or more amino acid substitutions to test for increased activity and improved specificity to 3,5,7-trioxododecanoyl-CoA or an analog thereof. In some cases, a “homolog” of the OAC SEQ ID NO: 1, is first identified. A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes that are orthologous can encode proteins with sequence similarity of about 45% to 100% amino acid sequence identity, and more preferably about 60% to 100% amino acid sequence identity. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, which may or may not be related to the original one.

Genes sharing a desired amount of identify (e.g., 45%, 50%, 55%, or 60% or greater) to the Cannabis sativa OAC, including homologs, orthologs, and paralogs, can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor.

Computational approaches to sequence alignment and determination of sequence identity include global alignments and local alignments. Global alignment uses global optimization to forces alignment to span the entire length of all query sequences. Local alignments, by contrast, identify regions of similarity within long sequences that are often widely divergent overall. For understanding the identity of a target sequence to the Cannabis sativa OAC template a global alignment can be used. Optionally, amino terminal and/or carboxy-terminal sequences of the target sequence that share little or no identity with the template sequence can be excluded for a global alignment and generation of an identity score.

Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 45% to 100% sequence identity. Proteins that are unrelated can have an identity which is essentially the same as would be expected to occur by chance if a database of sufficient size is scanned (about 5%).

Pairwise global sequence alignment can be carried out using Cannabis sativa OAC SEQ ID NO: 1 as the template. Alignment can be performed using the Needleman-Wunsch algorithm (Needleman, S. & Wunsch, C. A general method applicable to the search for similarities in the amino acid sequence of two proteins J. Mol. Biol, 1970, 48, 443-453) implemented through the BALIGN tool (http://balign.sourceforge.net/). Default parameters are used for the alignment and BLOSUM62 was used as the scoring matrix. The disclosure also relates to wild-type sequences previously annotated as “hypothetical protein” or “putative protein” and determined to be OAC homologs based on the current disclosure. Based in least on Applicant's identification, testing, motif identification, and sequence alignments (see FIG. 3), the current disclosure further allows for the identification of OAC suitable for use in engineered cells and methods of the disclosure, such as creating variants as described herein.

For the purpose of amino acid position numbering, SEQ ID NO: 1 is used as the reference sequence. For example, mention of amino acid position 79 is in reference to SEQ ID NO:1, but in the context of a different OAC sequence (a target sequence or other template sequence) the corresponding amino acid position for variant creation may have the same or different position number, (e.g. 78, 79 or 80). In some cases, the original amino acid and its position on the SEQ ID NO: 1 reference template will precisely correlate with the original amino acid and position on the target OAC. In other cases, the original amino acid and its position on the SEQ ID NO: 1 template will correlate with the original amino acid, but its position on the target will not be in the corresponding template position. However, the corresponding amino acid on the target can be a predetermined distance from the position on the template, such as within 10, 9, 8, 7, 6, 5, 4, 3, 2, or 1 amino acid positions from the template position. In other cases, the original amino acid on the SEQ ID NO: 1 template will not precisely correlate with the original amino acid on the target. However, one can understand what the corresponding amino acid on the target sequence is based on the general location of the amino acid on the template and the sequence of amino acids in the vicinity of the target amino acid, especially referring to the alignment provided in FIG. 3. It is understood that additional alignments can be generated with OAC sequences not specifically disclosed herein, and such alignments can be used to understand and generate new OAC variants in view of the current disclosure. In some modes of practice, the alignments can allow one to understand common or similar amino acids in the vicinity of the target amino acid, and those amino acids may be viewed as “sequence motif” having a certain amount of identity or similarity to between the template and target sequences. Those sequence motifs can be used to describe portions of OAC sequences where variant amino acids are located, and the type of variation(s) that can be present in the motif.

In some cases, it can be useful to use the Basic Local Alignment Search Tool (BLAST) algorithm to understand the sequence identity between an amino acid motif in a template sequence and a target sequence. Therefore, in preferred modes of practice, BLAST is used to identify or understand the identity of a shorter stretch of amino acids (e.g. a sequence motif) between a template and a target protein. BLAST finds similar sequences using a heuristic method that approximates the Smith-Waterman algorithm by locating short matches between the two sequences. The (BLAST) algorithm can identify library sequences that resemble the query sequence above a certain threshold. Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

FIG. 3 shows an alignment of SEQ ID NO: 1 (Cannabis sativa OAC) to other OAC homologs (SEQ ID NOs 2 and 3).

Methods known in the art can be used for the testing the enzymatic activity of OAC, and OAC variant enzymes, as well as OLS and OLS variant enzymes.

In some embodiments, an in vitro reaction composition will include an OAC or its variant (purified or in cell lysate or cell extract), and a 3,5,7-trioxoacyl-CoA or a 3,5,7-trioxocarboxylate substrate or an analog thereof, produced by OLS catalyzed reaction. The enzyme combination can convert the substrates to the desired product, e.g., olivetolic acid or its analogs or derivatives, or a combination thereof.

In some embodiments, an in vitro reaction composition will include the non-natural OAC and an a natural or non-natural OLS (purified or in cell lysate or cell extract), malonyl-CoA, and an acyl-CoA (non-limiting examples include acetyl-CoA, propionyl-CoA, butyryl-CoA, valeryl-CoA, hexanoyl-CoA, heptanoyl-CoA, octanoyl-CoA, nonanoyl-CoA, decanoyl-CoA, one or more of C12, C14, C16, C18, C20 or C22 chain length fatty acid CoA, an aromatic acid CoA, for example, benzoic, chorismic, phenylacetic and phenoxyacetic acid CoA, or its analogs), that can convert the substrates to the desired product, e.g., olivetolic acid or its analogs or derivatives, or a combination thereof.

In some embodiments, at least two-fold increase of enzymatic activity can be seen in in vitro reactions using cell lysates expressing OAC variants, or from purified preparations of the OAC variants (e.g., purified from cell lysates).

In some embodiments, when using cell lysates, cells expressing OAC variant and an a natural or variant OLS are treated by cell lysis agent (e.g., BPER II, BugBuster®), in the presence of protease inhibitors, 10 mM DTT, benzonase and lysozyme. The lysate is added to the substrates comprising one or more acyl-CoA and malonyl-CoA in the presence or absence of purified OAC enzyme to initiate reactions. Reactions can run for 30 minutes before quenching with formic acid-acidified 75% acetonitrile. Samples can be centrifuged to remove cellular debris and then analyzed for the products formed using LCMS. The rate of formation of OLA can be determined.

In some embodiments, OLS (natural and non-natural) and OAC (natural and non-natural) enzymes can work in coordination. In some embodiments, starting with malonyl-CoA and an acyl-CoA, natural and non-natural OLS can produce 3,5,7-trioxoacyl-CoA or 3,5,7-trioxocarboxylate. 3,5,7-trioxoacyl-CoA or 3,5,7-trioxocarboxylate can be converted by natural and non-natural OAC to 2,4-dihydroxy-6-alkylbenzoic acid. Additionally, 3,5,7-trioxoacyl-CoA or 3,5,7-trioxocarboxylate can be converted to olivetol or its analogs by natural and non-natural OLS. Thus, a ratio of olivetolic acid to olivetol formed can be indicative of the OAC activity and OLS activity. In some embodiments, a higher ratio of olivetolic acid to olivetol formed can be indicative of higher OAC activity. In some embodiments, at a given concentration of OLS, the rate of OAC can be expressed in terms of a ratio of olivetolic acid to olivetol formed/min/unit of OAC.

In some embodiments, at a given concentration of OLS, the rate can be expressed in terms of μM olivetolic acid/min/μμM OAC. In some embodiments, at a given concentration of OLS, the rate can be expressed in terms of mol of olivetolic acid/min/mol of OAC. In some embodiments, the rate can be expressed in terms of μmol of olivetolic acid/min/ng of OAC. In some embodiments, OAC and OLS provides a rate of formation of olivetolic acid of about 0.005 μM, 0.010 μM, 0.020 μM, 0.050 μM, 0.100 μM, 0.250 μM, 0.500 μM, 1 μM, 1.5 μM, 2 μM, 2.5 μM, 3 μM, 3.5 μM, 4 μM, 4.5 μM, 5 μM, 5.5 μM, 6 μM or greater olivetolic acid/min/μM enzyme.

Site-directed mutagenesis or sequence alteration (e.g., site-specific mutagenesis or oligonucleotide-directed) can be used to make specific changes to a target OAC and/or OLS DNA sequence to provide a variant DNA sequence encoding OAC and/or OLS with the desired amino acid substitution. As a general matter, an oligonucleotide having a sequence that provides a codon encoding the variant amino acid is used. Alternatively, artificial gene synthesis of the entire coding region of the variant OAC and/or OLS DNA sequence can be performed as preferred OAC and/or OLS targeted for substitution are generally less than 150 amino acids long.

Exemplary techniques using mutagenic oligonucleotides for generation of a variant OAC sequence include the Kunkel method which may utilize an OAC gene and/or OLS gene sequence placed into a phagemid. The phagemid in E. coli OAC ssDNA and/or OLS ssDNA which is the template for mutagenesis using an oligonucleotide which is a primer extended on the template.

Depending on the restriction enzyme sites flanking a location of interest in the OAC and/or OLS DNA, cassette mutagenesis may be used to create a variant sequence of interest. For cassette mutagenesis, a DNA fragment is synthesized inserted into a plasmid, cleaved with a restriction enzyme, and then subsequently ligated to a pair of complementary oligonucleotides containing the OAC and/or OLS variant mutation. The restriction fragments of the plasmid and oligonucleotide can be ligated to one another.

Another technique that can be used to generate the non-natural OAC and/or OLS sequence is PCR site directed mutagenesis. Mutagenic oligonucleotide primers are used to introduce the desired mutation and to provide a PCR fragment carrying the mutated sequence. Additional oligonucleotides may be used to extend the ends of the mutated fragment to provide restriction sites suitable for restriction enzyme digestion and insertion into the gene.

Commercial kits for site-directed mutagenesis techniques are also available. For example, the Quikchange™ kit uses complementary mutagenic primers to PCR amplify a gene region using a high-fidelity non-strand-displacing DNA polymerase such as pfu polymerase. The reaction generates a nicked, circular DNA which is relaxed. The template DNA is eliminated by enzymatic digestion with a restriction enzyme such as DpnI which is specific for methylated DNA.

In some embodiments, an expression vector or vectors can be constructed to include one or more non-natural OAC and/or OLS encoding nucleic acids as exemplified herein operably linked to regulatory element functional in the host organism. Expression vectors applicable for use in the microbial host organisms provided include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate regulatory element. Selectable marker genes also can be included that, for example, provide resistance to antibiotics or toxins, complement auxotrophic deficiencies, or supply critical nutrients not in the culture media. Regulatory element can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different regulatory element, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

An engineered cell can include one or more copies of a gene encoding the non-natural OAC. Optionally the engineered cell can include at least one copy of a gene encoding the non-natural OAC and at least one copy of a gene encoding a different OAC, for example, a wild type OAC, or a different (second) non-natural OAC with an amino acid variation that is different than the first non-natural OAC.

The expression of two different OAC alleles may lead to the formation of various dimeric forms of OAC, including homodimers and heterodimers. For example, the expression of an allele encoding a non-natural OAC (ᵥ) of the disclosure and an allele encoding a wild type OAC (_{wt}) may lead to the formation of the following dimers (two different homodimers, and two different heterodimers): aᵥbᵥ, a_{wt}b_{wt}, aᵥb_{wt}, and a_{wt}bᵥ. As another example, the expression of an allele encoding a first non-natural OAC (ᵥₗ) of the disclosure and an allele encoding a second non-natural OAC (ᵥ₂) may lead to the formation of the following dimers (two different homodimers, and two different heterodimers): aᵥ₁bᵥ₁, aᵥ₂bᵥ₂, aᵥ₁bᵥ₂, and aᵥ₂bᵥ₁. In embodiments, the presence of the amino acid variation in the non-natural OAC will not cause the non-natural OAC to lose its ability to dimerize.

Heterodimeric cyclases such as heterodimeric lycopene cyclases have been found in bacteria. For example, heterodimeric lycopene cyclase proteins CrtYc and crtYd have been found in Brevibacterium linens (Krubasik, P., and G. Sandmann (2000) Mol. Gen. Genet. 263:423-432), and also in from Mycobacterium aurum A+ (Viveiros, M., et al. (2000) FEMS Microbiol. Lett. 187:95-101.

As used herein the term “about” means ±10% of the stated value. The term “about” can mean rounded to the nearest significant digit. Thus, about 5% means 4.5% to 5.5%. Additionally, “about” in reference to a specific number also includes that exact number. For example, about 5% also includes exact 5%.

As used herein, the term “exogenous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

It is understood that when more than one exogenous nucleic acid is included in a microbial organism, the more than one exogenous nucleic acid(s) refers to the referenced encoding nucleic acid or biosynthetic activity, as discussed above. It is further understood, as disclosed herein, that more than one exogenous nucleic acid(s) can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

Exogenous variant OAC-encoding nucleic acid sequences can be introduced stably or transiently into a host cell using techniques well known in the art including, but not limited to, conjugation, electroporation, chemical transformation, transduction, transfection, and ultrasound transformation. Optionally, for exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffineister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

The term “isolated” when used in reference to a microbial organism is intended to mean an organism that is substantially free of at least one component that the referenced microbial organism is found with in nature. The term includes a microbial organism that is removed from some or all components as it is found in its natural environment. The term also includes a microbial organism that is removed from some or all components as the microbial organism is found in non-naturally occurring environments.

In some embodiments, the OAC variant gene is introduced into a cell with a gene disruption. The term “gene disruption,” or grammatical equivalents thereof, is intended to mean a genetic alteration that renders the encoded gene product inactive or attenuated. The genetic alteration can be, for example, deletion of the entire gene, deletion of a regulatory sequence required for transcription or translation, deletion of a portion of the gene which results in a truncated gene product, or by any of various mutation strategies that inactivate or attenuate the encoded gene product. One particularly useful method of gene disruption is complete gene deletion because it reduces or eliminates the occurrence of genetic reversions. The phenotypic effect of a gene disruption can be a null mutation, which can arise from many types of mutations including inactivating point mutations, entire gene deletions, and deletions of chromosomal segments or entire chromosomes. Specific antisense nucleic acid compounds and enzyme inhibitors, such as antibiotics, can also produce null mutant phenotype, therefore being equivalent to gene disruption.

A metabolic modification refers to a biochemical reaction that is altered from its naturally occurring state. Therefore, microorganisms may have genetic modifications to nucleic acids encoding metabolic polypeptides, or functional fragments thereof. Exemplary metabolic modifications are disclosed herein.

The microorganisms provided herein can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

A variety of microorganism may be suitable for incorporating the variant OAC, optionally with one or more other exogenous nucleic acid encoding one or more enzymes of the olivetolic acid pathway (such as OLS) or cannabigerol pathway. Such organisms include both prokaryotic and eukaryotic organisms. In some embodiments, the eukaryotic microorganisms include, but are not limited to yeast, fungi, plant, or algae. In some embodiments, the eukaryotic microorganisms include microalgae.

Nonlimiting examples of microalgae for incorporating the non-natural OAC, optionally with one or more other exogenous nucleic acid encoding one or more enzymes of the olivetolic acid pathway or cannabigerol pathway include members of the genera Amphora, Ankistrodesmus, Aplanochytrium, Asteromonas, Boekelovia, Bolidomonas, Borodinella; Botrydium, Botryococcus, Bracteococcus, Carteria, Chaetoceros, Chlamydomonas, Chlorella, Chlorococcum, Chlorogonium, Chrococcidiopsis, Chroomonas, Chrysophyceae, Chrysosphaera, Colwellia, Cricosphaera, Oypthecodinium, Cryptococcus, Cryptomonas, Cunninghamella, Cyclotella, Desmodesmus, Dunaliella, Elina, Ellipsoidon, Emiliania, Eremosphaera, Ernodesmius, Euglena, Eustigmatos, Fragilaria, Fragilariopsis, Franceia, Gloeothamnion, Haematococcus, Hantzschia, Heterosigma, Hymenomonas, Isochrysis, Japanochytrium, Labrinthula, Labyrinthomyxa, Labyrinthula, Lepocinclis, Micractinium, Monodus, Monoraphidium, Moritella, Mortierella, Mucor, Nannochloris, Nannochloropsis, Navicula, Neochloris, Nephrochloris, Nephroselmis, Nitzschia, Ochromonas, Oedogonium, Oocystis, Ostreococcus, Parachlorella, Parietochloris, Pascheria, Pavlova, Pelagomonas, Phaeodactylum, Phagus, Pichia, Picochlorum, Pithium, Platymonas, Pleurochrysis, Pleurococcus, Porphyridium, Prototheca, Pseudochlorella, Pseudoneochloris, Pseudostaurastrum, Pyramimonas, Pyrobotrys, Rhodosporidium, Scenedesmus, Schizochlamydella, Schizochytrium, Skeletonema, Spirulina, Spyrogyra, Stichococcus, Tetrachlorella, Tetraselmis, Thalassiosira, Thraustochytrium, Tribonema, Ulkenia, Vaucheria, Vibrio, Viridiella, Vischeria, and Volvox.

In some embodiments, the prokaryotic microorganisms include, but are not limited to bacteria, including archaea and eubacteria.

Exemplary microorganisms are reported in U.S. application Ser. No. 13/975,678 (filed Aug. 26, 2013), which is incorporated herein by reference in its entirety, and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum; Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Thermus thermophilus, Pseudomonas species, including Pseudomonas aeruginosa, Pseudomonas putida, Pseudomonas stutzeri, Pseudomonas fluorescens, Rhodobacter spaeroides, Thermoanaerobacter brockii, Metallosphaera sedula, Leuconostoc mesenteroides, Chloroflexus aurantiacus, Roseiflexus castenholzii, Erythrobacter, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Haloferax mediterranei, Agrobacterium tumefaciens, Achromobacter denitricans, Fusobacterium nucleatum, Streptomyces clavuligenus, Acinetobacter baumanii, Lachancea kluyveri, Trichomonas vaginalis, Trypanosoma brucei, Pseudomonas stutzeri, Bradyrhizobium japonicum, Mesorhizobium loti, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM20162, Cyanobium PCC7001, Dictyostelium discoideum AX4, as well as other exemplary species disclosed herein or available as source organisms for corresponding genes.

In certain embodiments, suitable organisms for incorporating the non-natural OAC include Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM3353, Flavobacterium frigoris, Fusobacterium nucleatum subsp. polymorphum ATCC 10953, Geobacillus sp. Y4.1MC1, Geobacillus themodenitrificans NG80-2, Geobacter bemidjiensis Bem, Geobacter. sulfurreducens, Geobacter sulfurreducens PCA, Geobacillus stearothermophilus DSM 2334, Haemophilus influenzae, Helicobacter pylori, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JCJ DSM3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC:33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, and Yersinia intermedia.

Eukaryotic and prokaryotic host cells can be engineered to comprise non-natural OAC. In some embodiments, the non-natural OAC can be expressed from an exogenous nucleic acid, and under control of regulatory elements that allow desired expression of the non-natural OAC in the cell. The non-natural OAC can be a part of a “pathway” that leads from one desired chemical substrate to a target chemical product. As such, in addition to the non-natural OAC, one or more other enzymes can be a part of a pathway and can function (a) “upstream” of the non-natural OAC, (b) “downstream” of the non-natural OAC, or both (a) and (b). The other pathway enzymes can be endogenous to the host cell, or can be introduced exogenously. Exemplary additional pathway enzymes include olivetol synthase which can function upstream, or concurrently, with the non-natural OAC. Other upstream enzymes can promote the formation of an alkanoyl-CoA substrate, such as hexanoyl-CoA, which is formed from hexanoic acid using hexanoyl-CoA synthetase. Yet other enzymes that can function upstream to the non-natural OAC are those involved in fatty acid biosynthesis.

Downstream enzymes include those that are active on a product of the non-natural OAC, or a derivative or analog thereof, or that provide substrate compounds that can be used to modify the product of the non-natural OAC. Exemplary enzymes include aromatic prenyltransferases which can add a partially saturated carbon chain to a carbon position on the product of the aromatic ring of the OAC product, 2,4-dihydroxy-6-alkylbenzoic acid, to form a cannabinoid. Downstream enzymes also include cannabinoid synthases which can promote formation of certain cannabinoid species. Other useful enzymes can form substrates useful for cannabinoid formation, such as substrates like geranyl pyrophosphate (GPP) formed using GPP synthase, wherein GPP can provide a partially saturated carbon chain for modification of the 2,4-dihydroxy-6-alkylbenzoic acid. GPP formation stems from the mevalonate pathway (MVA) or methylerythritol-4-phosphate (MEP) pathway, which produce isopentyl pyrophosphate (IPP) and dimethylallyl pyrophosphate (DMAPP), which are precursors to GPP. In some embodiments, GPP is formed from prenol or isoprenol using an alternative non-MEP, non-MVA geranyl pyrophosphate pathway. The pathway comprises alcohol kinase, alcohol diphosphate kinase, phosphate kinase, isopentenyl diphosphate isomerase, and geranyl pyrophosphate synthase enzymes

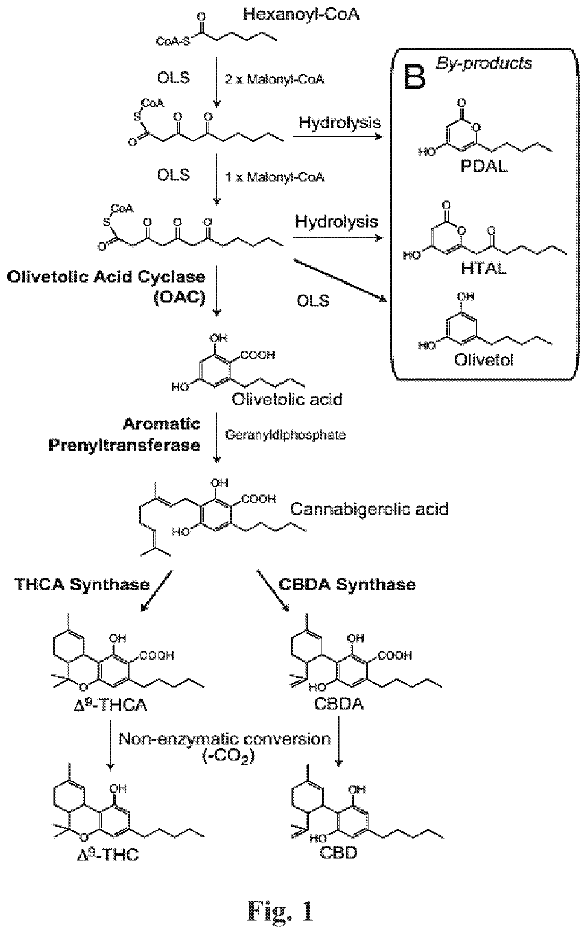

FIG. 1 shows exemplary pathways to CBGA formation from malonyl-CoA, hexanoyl-CoA, and geranyl diphosphate. In some cases, the engineered cell of the disclosure can utilize hexanoyl-CoA that is produced from a cellular fatty acid biosynthesis pathway. For example, hexanoyl-CoA can be formed endogenously via reverse beta-oxidation of fatty acids.

In other embodiments, the engineered cell can further include hexanoyl-CoA synthetase, such as encoded by an exogenous nucleic acid. Exemplary hexanoyl-CoA synthetase genes include enzymes endogenous to bacteria, including E. coli, as well as eukaryotes, including yeast and C. sativa (see for example Stout et al., Plant J., 2012; 71:353-365, which is incorporated by reference in its entirety). Endogenous malonyl-CoA formation can be supplemented by formation from acetyl CoA using overexpression of acetyl-CoA carboxylase. Accordingly, the engineered cell can further include acetyl-CoA carboxylase, such as expressed on a transgene or integrated into the genome.

Acetyl-CoA carboxylase (EC 6.4.1.2) catalyzes the ATP-dependent carboxylation of acetyl-CoA to malonyl-CoA. This enzyme is biotin dependent and is the first reaction of fatty acid biosynthesis initiation in several organisms. Exemplary enzymes are encoded by accABCD of E. coli (Davis et al, J Biol Chem 275:28593-8 (2000)), ACC1 of Saccharomyces cerevisiae and homologs (Sumper et al, Methods Enzym 71:34-7 (1981), which is incorporated by reference in its entirety).

FIG. 1 also shows prenyltransferase converts OLA and GPP to CBGA. Accordingly, the engineered cell can further include prenyltransferase, such as expressed on a transgene or integrated into the genome.

Optionally, the engineered cell can include one or more exogenous genes which allow the cell to grow on carbon sources the cell would not normally metabolize, or one or more exogenous genes or modifications to endogenous genes that allow the cell to have improved growth on carbon sources the cell normally uses. For example, WO2015/051298 (MDH variants) and WO2017/075208 (MDH fusions) describe genetic modifications that provide pathways allowing to cell to grow on methanol; WO2009/094485 (syngas) describes genetic modifications that provide pathways allowing to cell to grow on synthesis gas.

In some embodiments, the engineered cell may further comprise enzymes for geranyl phosphate pathways. For example, MVP pathway, MEP pathway, non-MVP, non-MEP pathways using isoprenol and prenol as precursors for the synthesis of geranyl pyrophosphate as disclosed in PCT application publication WO2017161041, which is incorporated by reference in its entirety. The alternative non-MEP, non-MVA geranyl pyrophosphate pathway comprises alcohol kinase, alcohol diphosphate kinase, phosphate kinase, isopentenyl diphosphate isomerase, and geranyl pyrophosphate synthase enzymes.

As used herein, the term “conservative substitution” refers to conservatively modified variants The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

As used herein, the term “bioderived” means derived from or synthesized by a biological organism and can be considered a renewable resource since it can be generated by a biological organism. Such a biological organism, in particular the microbial organisms disclosed herein, can utilize feedstock or biomass, such as, sugars or carbohydrates obtained from an agricultural, plant, bacterial, or animal source. Alternatively, the biological organism can utilize atmospheric carbon. As used herein, the term “biobased” means a product as described above that is composed, in whole or in part, of a bioderived compound of the disclosure. A biobased or bioderived product is in contrast to a petroleum derived product, wherein such a product is derived from or synthesized from petroleum or a petrochemical feedstock.

The cell cultures include engineered cells as disclosed herein that produce olivetolic acid, analogs and derivative of olivetolic, acid and/or one or more cannabinoids or analogs or derivatives of the cannabinoids in a culture medium that includes a carbon source that can also be an energy source, such as glycerol, a sugar, a sugar alcohol, a polyol, an organic acid, or an amino acid. In various embodiments, the culture medium can include at least one feed molecule, including but not limited to, one or more organic acids, amino acids, or alcohols that can be converted into a precursor of a cannabinoid, cannabinoid analog, olivetolic acid, or an olivetolic acid precursor (e.g., acetyl-CoA, malonyl-CoA, hexanoyl-CoA, or other acyl-CoA molecules), or geranyldiphosphate).

In certain embodiments of any of the foregoing or following, the suitable medium comprises a fermentable sugar. In some embodiments, the suitable medium comprises a pretreated cellulosic feedstock. In certain embodiments of any of the foregoing or following, the suitable medium comprises a non-fermentable carbon source. In some embodiments, the non-fermentable carbon source comprises ethanol. Examples of feed molecules include, but are not limited to, bicarbonate, acetate, malonate, oxaloacetate, aspartate, glutamate, beta-alanine, alpha-alanine, a fatty acid (or its conjugate base, such as hexanoate, butyrate, pentanoate, heptanoate, octanoate, decanoate, C11-C30 fatty acids, 2-methyl hexanoate, 4-methyl hexanoate, 4-methyl hexanoate, 2-hexanoate, 3-hexanoate, 5-hexanoate, 5-chloro pentanoate, 5-(methyl sulfanyl pentanoate, etc.), a fatty alcohol (e.g., a fatty alcohol of chain length C2-C22, a C2, C3, C4, C5, C7, C8, C10, C12, C14, C16, C18, C20, C22, or longer chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids, etc.), prenol, isoprenol and geraniol. Accordingly, “fatty acid” or “carboxylic acid” as used throughout herein includes acetate, propionate, butyrate, hexanoate, pentanoate, heptanoate, octonoate, decanoate, valerate, or isovalerate, a fatty acid of a chain length other than C6, a fatty acid of chain length C2-C30, including odd and even chain lengths, a C2, C4, C3, C5, C7, C8, C10, C12, C14, C16, C18, C20, C22, or longer chain length fatty acid, and an aromatic acid, for example benzoic, chorismic, phenylacetic and phenoxyacetic acids. Accordingly, “fatty alcohol” as used throughout herein includes a fatty alcohol of chain length C2-C22, a C2, C3, C4, C5, C7, C8, C10, C12, C14, C16, C18, C20 or C22 chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids, etc. In various embodiments, one, two, three, or more feed molecules can be present in the culture medium during at least a portion of the time the culture is producing olivetolic acid or a derivative thereof or a cannabinoid. Alternatively, or in addition, the culture medium can include a supplemental compound that can be a cofactor, or a precursor of a cofactor used by an enzyme that functions in a cannabinoid pathway, such as, for example, biotin, thiamine, pantothenate, or 4-phosphopantetheine. A culture medium in some embodiments can include one or more inhibitors of one or more enzymes, such as an enzyme that functions in fatty acid biosynthesis, such as but not limited to cerulenin, thiolactomycin, triclosan, diazaborines such as thienodiazaborine, isoniazid, and analogs thereof.

In some modes of practice, one or more feed molecule(s) is provided to the cell culture to serve as precursor compound(s) so desired amounts of malonyl-CoA and acyl-CoA substrates become present in the cell. For example, providing a feed of a selected fatty acid or selected fatty alcohol can serve as a precursor to formation of a desired acyl-CoA substrate, and in turn the amount of desired acyl-CoA substrate can be increased relative to malonyl-CoA. Subsequently, a desired ratio of malonyl-CoA to acyl-CoA can be beneficial for forming 2,4-dihydroxy-6-alkylbenzoic acid in conjunction with OLS and the non-natural OAC. In modes of practice, the method includes providing a feed of one or more precursor compounds to the cell culture so the molar ratio of malonyl-CoA to acyl-CoA in the cell is in the range of about 500:1 to about 1:500, about 250:1 to about 1:250, about 150:1 to about 1:150, about 100:1 to about 1:100, about 75:1 to about 1:75, about 50:1 to about 1:50, about 25:1 to about 1:25, about 15:1 to about 1:15, or about 10:1 to about 1:10.

Further, the engineered cell can further include one or more enzymes of a cellular fatty acid biosynthesis pathway to promote conversion of the feed molecule(s) to a desired acyl-CoA substrate. As noted herein, exemplary enzymes include hexanoyl-CoA synthetase and/or acetyl-CoA carboxylase to promote conversion of feed compounds, such as fatty acids and fatty alcohols.

Further provided are methods for producing cannabinoids that include culturing a cell engineered for the production of olivetolic acid or a derivative thereof or a cannabinoid as provided herein under conditions in which the cell produces olivetolic acid, a derivative thereof, or a cannabinoid. In some examples, the methods include culturing the engineered cells in a culture medium that includes at least one feed molecule or supplement such as but not limited to: bicarbonate, acetate, malonate, oxaloacetate, aspartate, glutamate, beta-alanine, alpha-alanine, a fatty acid (or its conjugate base, such as hexanoate, butyrate, pentanoate, heptanoate, octanoate, decanoate, etc.), a fatty alcohol (includes a fatty alcohol of chain length C2-C22, a C2, C3, C4, C5, C7, C8, C10, C12, C14, C16, C18, C20 or C22 chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids), prenol, isoprenol, geraniol, biotin, thiamine, pantothenate, and 4-phosphopantetheine in the culture medium during at least a portion of the culture period when the cells are producing olivetolic acid, a derivative thereof, or a cannabinoid. Alternatively, or in addition, the methods can optionally include adding one or more fatty acid biosynthesis inhibitors to the culture medium during at least a portion of the culture period when the cells are producing olivetolic acid or a derivative thereof or a cannabinoid. The methods can further include recovering olivetolic acid or a derivative thereof or at least one cannabinoid from the cell, the culture medium, or whole culture. Also provided are cannabinoids produced by the methods provided herein, including derivatives of naturally-occurring cannabinoids, such as, but not limited to, cannabinoid derivatives having different acyl chain lengths than are found in naturally-occurring cannabinoids. The term “derivative” as used herein includes but is not limited to analogs.

In some embodiments, the cells provided herein that are engineered to produce olivetolic acid or a derivative thereof or a cannabinoid are further engineered to increase the production of the olivetolic acid, olivetolic acid derivative, or cannabinoid product, for example by increasing metabolic flux to a cannabinoid or olivetolic acid pathway, or by decreasing byproduct formation.

A cell engineered to produce olivetolic acid, an analog or derivative of olivetolic acid, or a cannabinoid, its analog or derivative is further engineered to increase the supply of coenzyme A (CoA) to increase its availability for producing acetyl-CoA and/or malonyl-CoA as well as hexanoyl-CoA or an alternative acyl-CoA.

Depending on the desired microorganism or strain to be used, the appropriate culture medium may be used. For example, descriptions of various culture media may be found in “Manual of Methods for General Bacteriology” of the American Society for Bacteriology: (Washington D.C., USA, 1981). As used here, “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements.

Exemplary carbon sources include sugar carbons such as sucrose, glucose, galactose, fructose, mannose, isomaltose, xylose, maltose, arabinose, cellobiose and 3-, 4-, or 5-oligomers thereof. Other carbon sources include alcohol carbon sources such as methanol, ethanol, glycerol, formate and fatty acids. Still other carbon sources include carbon sources from gas such as synthesis gas, waste gas, methane, CO, CO₂ and any mixture of CO, CO₂ with H₂. Other carbon sources can include renewal feedstocks and biomass. Exemplary renewal feedstocks include cellulosic biomass, hemicellulosic biomass and lignin feedstocks.

In some embodiments, culture conditions include aerobic, anaerobic or substantially anaerobic growth or maintenance conditions. Exemplary anaerobic conditions have been described previously and are well known in the art. Exemplary anaerobic conditions for fermentation processes are disclosed, for example, in U.S. Patent Application Publication No 2009/0047719, filed Aug. 10, 2007. Any of these conditions can be employed with the microbial organisms as well as other anaerobic conditions well known in the art.

The culture conditions can include, for example, liquid culture procedures as well as fermentation and other large scale culture procedures. Useful yields of the products can be obtained under aerobic, anaerobic or substantially anaerobic culture conditions.

An exemplary growth condition for achieving, one or more cannabinoid product(s) includes anaerobic culture or fermentation conditions. In certain embodiments, the microbial organism can be sustained, cultured or fermented under anaerobic or substantially anaerobic conditions. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

The culture medium at the start of fermentation may have a pH of about 5 to about 7. The pH may be less than 11, less than 10, less than 9, or less than 8. In other embodiments the pH may be at least 2, at least 3, at least 4, at least 5, at least 6, or at least 7. In other embodiments, the pH of the medium may be about 6 to about 9.5; 6 to about 9, about 6 to 8 or about 8 to 9.

Suitable purification and/or assays to test, e.g., for the production of 3-geranyl-olivetolate can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

The 3-geranyl-olivetolate (CBGA) or other target molecules may be separated from other components in the culture using a variety of methods well known in the art. Such separation methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. All of the above methods are well known in the art.

The disclosure also contemplates methods for, generally, forming an aromatic compound. The method involves contacting three molecules of malonyl-CoA and one molecule of hexanoyl-CoA to form an aromatic compound. For example, in particular, the disclosure contemplates use of various acyl-CoA substrates such as acetyl-CoA, propionyl-CoA, butyryl-CoA, valeryl-CoA, hexanoyl-CoA, heptanoyl-CoA, nonanoyl-CoA, decanoyl-CoA, one or more of C12, C14, C16, C18, C20 or C22 chain length fatty acid CoA, an aromatic acid CoA, for example, benzoic, chorismic, phenylacetic and phenoxyacetic acid CoA in such an olivetol synthase and OAC-catalyzed reaction. The method can be performed in vivo (e.g., within the engineered cell) or in vitro.

The disclosure also contemplates methods for forming a prenylated aromatic compound. The method can be performed in vivo (e.g., within the engineered cell) or in vitro. In view of the improved specificity of the OAC variants, the disclosure also provides compositions that are enriched for the precursors for the desired cannabinoids, analogs and derivatives thereof, or combinations thereof.

In particular, the disclosure provides compositions enriched for olivetolic acid, analogs and derivatives of olivetolic acid. The nature of the olivetolic acid analogs will depend on the initial acyl-CoA substrate, e.g., acetyl-CoA, propionyl-CoA, butyryl-CoA, valeryl-CoA, hexanoyl-CoA, heptanoyl-CoA, octanoyl-CoA, nonanoyl-CoA, decanoyl-CoA, one or more of C12, C14, C16, C18, C20 or C22 chain length fatty acid CoA, an aromatic acid CoA, for example, benzoic, chorismic, phenylacetic and phenoxyacetic acid CoA.

The chemical structures and pathways for producing olivetolic acid and its analogs, cannabigerolic acid and its analogs, and cannabigerol and its analogs are shown in FIG. 5.

The olivetolic acid, analogs and derivatives of olivetolic acid can serve as a substrate for aromatic prenyltransferase and to produce cannabigerolic acid (CBGA) and its analogs and derivatives. CBGA and its analogs and derivatives can be decarboxylated either enzymatically, catalytically or thermally (by heat) to cannabigerol (CBG) and its analogs and derivatives.

As used herein, the terms “cannabinoid”, “cannabinoid product”, and “cannabinoid compound” or “cannabinoid molecule” are used interchangeably to refer a molecule containing a polyketide moiety, e.g., olivetolic acid or another 2-alkyl-4,6-dihydroxybenzoic acid, and a terpene-derived moiety e.g., a geranyl group. Geranyl groups are derived from the diphosphate of geraniol, known as geranyl-diphosphate or geranyl-pyrophosphate that forms the acidic cannabinoid cannabigerolic acid (CBGA). CBGA can be converted to further bioactive cannabinoids both enzymatically (e.g., by decarboxylation via enzyme treatment in vivo or in vitro to form the neutral cannabinoid cannabigerol), catalytically or thermally (e.g., by heating).

The term cannabinoid includes acid cannabinoids and neutral cannabinoids. The term cannabinoids also includes derivatives and analogs of naturally-occurring cannabinoids, such as, but not limited to, cannabinoids having different alkyl chain lengths of side groups than are found in naturally-occurring cannabinoids. The term “acidic cannabinoid” generally refers to a cannabinoid having a carboxylic acid moiety. The carboxylic acid moiety may be present in protonated form (i.e., as —COOH) or in deprotonated form (i.e., as carboxylate —COO⁻). Examples of acidic cannabinoids include, but are not limited to, cannabigerolic acid, cannabidiolic acid, and Δ⁹-tetrahydrocannabinolic acid. The term “neutral cannabinoid” refers to a cannabinoid that does not contain a carboxylic acid moiety (i.e., does contain a moiety —COOH or —COO⁻). Examples of neutral cannabinoids include, but are not limited to, cannabigerol, cannabidiol, and Δ⁹-tetrahydrocannabinol.

Cannabinoids may include, but are not limited to, cannabichromene (CBC), cannabichromenic acid (CBCA), cannabigerol (CBG), cannabigerolic acid (CBGA), cannabidiol (CBD), cannabidiolic acid (CBD), Δ9-trans-tetrahydrocannabinol (Δ9-THC), Δ9-tetrahydrocannabinolic acid (THCA), Δ8-trans-tetrahydrocannabinol (Δ8-THC), cannabicyclol (CBL), cannabielsoin (CBE), cannabinol (CBN), cannabinodiol (CBND), cannabitriol (CBT), cannabigerolic acid monomethylether (CBGAM), cannabigerol monomethylether (CBGM), cannabigerovarinic acid (CBGVA), cannabigerovarin (CBGV), cannabichromenic acid (CBCA), cannabichromevarinic acid (CBCVA), cannabichromevarin (CBCV), cannabidiol monomethylether (CBDM), cannabidiol-C4 (CBD-C4), cannabidivarinic acid (CBDVA), cannabidivarin (CBDV), cannabidiorcol (CBD-C1), Δ9-tetrahydrocannabinolic acid A (THCA-A), Δ9-tetrahydrocannabinolic acid B (THCA-B), Δ9-tetrahydrocannabinol (THC), Δ9-tetrahydrocannabinolic acid-C4 (THCA-C4), Δ9-tetrahydrocannabinol-C4 (THC-C4), Δ9-tetrahydrocannabivarinic acid (THCVA), Δ9-tetrahydrocannabivarin (THCV), Δ9-tetrahydrocannabiorcolic acid (THCA-C1), Δ9-tetrahydrocannabiorcol (THC-C1), Δ7-cis-iso-tetrahydrocannabivarin, Δ8-tetrahydrocannabinolic acid (Δ8-THCA), Δ8-tetrahydrocannabinol (Δ8-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN-C4), cannabivarin (CBV), cannabinol-C2 (CNB-C2), cannabiorcol (CBN-C1), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

Cannabigerolic acid (CBGA) has the following chemical names (E)-3-(3,7-dimethyl-2,6-octadienyl)-2,4-dihydroxy-6-pentylbenzoic acid, and 3-[(2E)-3,7-dimethylocta-2,6-dien-1-yl]-2,4-dihydroxy-6-pentylbenzoic acid, and the following chemical structure:

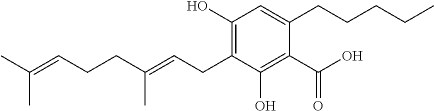 [SMILES: CCCCCc1cc(O)c(C/C=C(\C)CCC=C(C)C)c(O)c1C(=O)O]

Additional cannabinoid analogs and derivatives that can be produced with the methods or the engineered host cells of the present disclosure may also include, but are not limited to, 2-geranyl-5-pentyl-resorcylic acid, 2-geranyl-5-(4-pentynyl)-resorcylic acid, 2-geranyl-5-(trans-2-pentenyl)-resorcylic acid, 2-geranyl-5-(4-methylhexyl)-resorcylic acid, 2-geranyl-5-(5-hexynyl) resorcylic acid, 2-geranyl-5-(trans-2-hexenyl)-resorcylic acid, 2-geranyl-5-(5-hexenyl)-resorcylic acid, 2-geranyl-5-heptyl-resorcylic acid, 2-geranyl-5-(6-heptynoic)-resorcylic acid, 2-geranyl-5-octyl-resorcylic acid, 2-geranyl-5-(trans-2-octenyl)-resorcylic acid, 2-geranyl-5-nonyl-resorcylic acid, 2-geranyl-5-(trans-2-nonenyl) resorcylic acid, 2-geranyl-5-decyl-resorcylic acid, 2-geranyl-5-(4-phenylbutyl)-resorcylic acid, 2-geranyl-5-(5-phenylpentyl)-resorcylic acid, 2-geranyl-5-(6-phenylhexyl)-resorcylic acid, 2-geranyl-5-(7-phenylheptyl)-resorcylic acid, (6aR,10aR)-1-hydroxy-6,6,9-trimethyl-3-propyl-6a,7,8,10a-tetrahydro-6H-dibenzo[b,d]pyran-2-carboxylic acid, (6aR,10aR)-1-hydroxy-6,6,9-trimethyl-3-(4-methylhexyl)-6a,7,8,10a-tetrahydro-6H-dibenzo[b,d]pyran-2-carboxylic acid, (6aR,10aR)-1-hydroxy-6,6,9-trimethyl-3-(5-hexenyl)-6a,7,8,10a-tetrahydro-6H-dibenzo[b,d]pyran-2-carboxylic acid, (6aR,10aR)-1-hydroxy-6,6,9-trimethyl-3-(5-hexenyl)-6a,7,8,10a-tetrahydro-6H-dibenzo[b,d]pyran-2-carboxylic acid, (6aR,10aR)-1-hydroxy-6,6,9-trimethyl-3-(6-heptynyl)-6a,7,8,10a-tetrahydro-6H-dibenzo[b,d]pyran-2-carboxylic acid, 3-[(2E)-3,7-dimethylocta-2,6-dien-1-yl]-6-(hexan-2-yl)-2,4-dihydroxybenzoic acid, 3-[(2E)-3,7-dimethylocta-2,6-dien-1-yl]-2,4-dihydroxy-6-(2-methylpentyl)benzoic acid, 3-[(2E)-3,7-dimethylocta-2,6-dien-1-yl]-2,4-dihydroxy-6-(3-methylpentyl)benzoic acid, 3-[(2E)-3,7-dimethylocta-2,6-dien-1-yl]-2,4-dihydroxy-6-(4-methylpentyl)benzoic acid, 3-[(2E)-3,7-dimethylocta-2,6-dien-1-yl]-2,4-dihydroxy-6-[(1E)-pent-1-en-1-yl]benzoic acid, 3-[(2E)-3,7-dimethylocta-2,6-dien-1-yl]-2,4-dihydroxy-6-[(2E)-pent-2-en-1-yl]benzoic acid, 3-[(2E)-3,7-dimethylocta-2,6-dien-1-yl]-2,4-dihydroxy-6-[(2E)-pent-3-en-1-yl]benzoic acid, 3-[(2E)-3,7-dimethylocta-2,6-dien-1-yl]-2,4-dihydroxy-6-(pent-4-en-1-yl)benzoic acid, 3-[(2E)-3,7-dimethylocta-2,6-dien-1-yl]-2,4-dihydroxy-6-propylbenzoic acid, 3-[(2E)-3,7-dimethylocta-2,6-dien-1-yl]-2,4-dihydroxy-6-butylbenzoic acid, 3-[(2E)-3,7-dimethylocta-2,6-dien-1-yl]-2,4-dihydroxy-6-hexylbenzoic acid, 3-[(2E)-3,7-dimethylocta-2,6-dien-1-yl]-2,4-dihydroxy-6-heptylbenzoic acid, 3-[(2E)-3,7-dimethylocta-2,6-dien-1-yl]-2,4-dihydroxy-6-octylbenzoic acid, 3-[(2E)-3,7-dimethylocta-2,6-dien-1-yl]-2,4-dihydroxy-6-nonanylbenzoic acid, 3-[(2E)-3,7-dimethylocta-2,6-dien-1-yl]-2,4-dihydroxy-6-decanylbenzoic acid, 3-[(2E)-3,7-dimethylocta-2,6-dien-1-yl]-2,4-dihydroxy-6-undecanylbenzoic acid, 6-(4-chlorobutyl)-3-[(2E)-3,7-dimethylocta-2,6-dien-1-yl]-2,4-dihydroxybenzoic acid, 3-[(2E)-3,7-dimethylocta-2,6-dien-1-yl]-2,4-dihydroxy-6-[4-(methylsulfanyl)butyl]benzoic acid, and others as listed in Bow, E. W. and Rimoldi, J. M., “The Structure-Function Relationships of Classical Cannabinoids: CB1/CB2 Modulation,” Perspectives in Medicinal Chemistry 2016:817-39 doi: 10.4137/PMC.S32171, incorporated by reference herein.

Cannabinoid precursor analogs and derivatives that can be produced with the methods or genetically modified host cells of the present disclosure may also include, but are not limited to, divarinolic acid, 5-pentyl-resorcylic acid, 5-(4-pentynyl)-resorcylic acid, 5-(trans-2-pentenyl)-resorcylic acid, 5-(4-methylhexyl)-resorcylic acid, 5-(5-hexynyl)-resorcylic acid, 5-(trans-2-hexenyl)-resorcylic acid, 5-(5-hexenyl)-resorcylic acid, 5-heptyl-resorcylic acid, 5-(6-heptynoic)-resorcylic acid, 5-octyl-resorcylic acid, 5-(trans-2-octenyl)-resorcylic acid, 5-nonyl-resorcylic acid, 5-(trans-2-nonenyl)-resorcylic acid, 5-decyl-resorcylic acid, 5-(4-phenylbutyl)-resorcylic acid, 5-(5-phenylpentyl)-resorcylic acid, 5-(6-phenylhexyl)-resorcylic acid, and 5-(7-phenylheptyl)-resorcylic acid.

### Example 1: Structural Analysis

Crystal structures of the OAC apo (PDB ID: 5B08), OAC-OLA binary complex (PDB ID: 5B09), and seven variants with single point mutations (PDB IDs: 5BOA-G) have been solved and are described by Yang et al. (FEBS Journal 283:1088-1106; 2016). OAC crystal structure information was used for further analysis involving examination of different linear tetraketide substrates in the OAC active site to identify catalytically-relevant residues.

Discovery Studio from Biovia was used to dock three versions of the linear tetraketide into the OAC apo structure: 3,5,7-trioxododecanoyl-CoA (LTKCoA), 3,5,7-trioxododecanoic acid (LTKacid), and 3,5,7-trioxododecanoate (LTKate). The OAC active site was identified using the Define Site From Receptor Cavities tool and verified that it included the volume where OLA is bound in the binary complex. The site sphere was expanded for docking LTKCoA due to its large size. The three ligands were docked into the OAC apo structure using CDOCKER with 10 random substrate binding conformations of the ligands and 10 top hits as output. The output substrate binding conformations were manually compared to bound OLA from the binary complex to verify which substrate binding conformations were likely to be catalytically relevant (pentyl group facing into the active site). While the majority of the LTKacid (6/10) and LTKate (9/10) substrate binding conformations were catalytically relevant, only 3/10 of the LTKCoA substrate binding conformations were. And while these 3 substrate binding conformations were selected as catalytically relevant due to the pentyl group facing into the active site, this group did not penetrate as far into the active site as that of the bound OLA ligand or the catalytically relevant LTKacid and LTKate substrate binding conformations. Residues within 5 Å of catalytically relevant and all other substrate binding conformations were identified. All residues within 5 Å of OLA were also within 5 Å of catalytically relevant substrate binding conformations. Included among these are residues Y72 and His78 which have been identified as the catalytic residues. Results are shown in Table 5.

TABLE 5

| | -CDOCKER Energy | | |
|---|---|---|---|
| Pose | LTKCoA | LTKacid | LTKate |
| 1 | 88.9514 | 47.4166 | 56.8703 |
| 2 | 88.2941 | 47.2513 | 55.7755 |
| 3 | 86.9624 | 47.2095 | 55.142 |
| 4 | 85.3544 | 46.8221 | 54.6999 |
| 5 | 85.2898 | 46.5224 | 54.5564 |
| 6 | 84.8627 | 46.1847 | 53.9633 |
| 7 | 84.3414 | 45.2931 | 53.8944 |
| 8 | 84.1242 | 45.2822 | 53.5068 |
| 9 | 83.6905 | 44.5590 | 53.4123 |
| 10 | 88.9514 | 44.5583 | 53.1857 |

Relative energy of the top 10 docking substrate binding conformations of each ligand with catalytically relevant substrate binding conformations in bold. Residues within 5 Å of docked substrate binding conformations are as follows: H5, I7, L9, F23, F24, Y27, V59, V61, V66, E67, I69, Q70, I73, I74, V79, G80, F81, G82, D83, R86, W89, L92, I94, D96, V46*, T47*, Q48*, K49*, N50*, and K51*, wherein the “*” indicates residues from chain B of OAC dimer.

Residues near catalytically relevant substrate binding conformations are as follows H5, I7, L9, F23, F24, Y27, V59, V66, I69, Q70, I73, I74, V79, G80, F81, G82, D83, R86, W89, L92, I94, D96, V46*, T47*, Q48*, K49*, and K51*, and wherein the “*” indicates amino acid residues from chain B of OAC dimer and corresponding to SEQ ID NO: 1. Identified residues include the catalytic residues Y72 and His78.

### Example 2: Variant Design

Amino acid variations introduced into OAC have amino acids with side chains that are similar to the wild type amino acid side chains and are designed to increase OAC production of OLA (see Table 1). The interior of the active site binds the pentyl-group of the tetraketide substrate and resulting product is lined mostly with amino acids with hydrophobic side chains. Substitutions at these positions with other amino acids with hydrophobic side chains are designed to alter binding of the pentyl-group and in turn provide higher OLA production. Residues outside and at the entrance to the active site are involved with binding of the ketone groups and CoA of the substrate. Substitutions at these positions with other amino acids with side chains with similar biochemical properties (hydrophobic, polar, charged, etc.) are designed to alter binding of the substrate and provide higher OLA production.

The amino acid positions shown in the Table 1 of OAC corresponds to SEQ ID NO: 1. It is expressly contemplated that the amino acid sequence of the non-natural OAC can have one or more amino acid variations at equivalent positions corresponding to any homolog of SEQ ID NO: 1.

Analog products formed by the OLS using varied starter molecules will differ at the pentyl-group portion of the molecule. Substitutions at residues of OAC that interact with this region of the substrate are designed to alter OAC production of analog products (see Table 6). Both the size and biochemical properties of the starter molecule used to produce the analog will dictate the type of amino acid variations in OAC that are designed to increase specificity towards the substrate. Analogs produced with large hydrophobic starter molecules such as CoA-bound aliphatic chains or aromatic rings are designed to having improved binding by amino acids with small hydrophobic side chains such as glycine, alanine, valine, leucine, isoleucine, or proline. Conversely, analogs produced with smaller hydrophobic starter molecule are designed to have improved binging by amino acids with large hydrophobic side chains such as methionine, phenylalanine, or tryptophan. Analogs produced with polar or charged starter molecules are designed to benefit from amino acids with polar side chains such as serine, threonine, cysteine, tyrosine, histidine, glutamine, or asparagine as well as charged side chains such as aspartic acid, glutamic acid, lysine, and arginine.

The amino acid positions shown in the Table 6 below of OAC corresponds to SEQ ID NO: 1. It is expressly contemplated that the amino acid sequence of the non-natural OAC can have one or more amino acid variations at equivalent positions corresponding to any homolog of SEQ ID NO: 1.

TABLE 6

| | Analogs with | Analogs with | Analogs with |
|---|---|---|---|
| | larger, hydrophobic | smaller, | polar or |
| | starter | hydrophobic starter | charged starter |
| Position | molecules | molecules | molecules |
| H5 | G,A,C,P,V | V,M,F,Y,W,Q,E,K,R | S,T,Y,N,Q, |
| | | | D,E,K,R |
| I7 | G,A,C,P,V,L,M | L,M,F,Y,W,K,R | S,T,Y,H,N,Q, |
| | | | D,E,K,R |
| L9 | G,A,C,P,V,I,M | I,M,F,Y,W,K,R | S,T,Y,H,N,Q, |
| | | | D,E,K,R |
| F23 | G,A,C,P,V,L,I,M,Y,W, | Y,W | S,T,Y,H,N,Q, |
| | S,T,H,N,Q,D,E,K,R | | D,E,K,R |
| F24 | G,A,C,P,V,L,I,M,Y,W, | Y,W | S,T,Y,H,N,Q, |
| | S,T,H,N,Q,D,E,K,R | | D,E,K,R |
| Y27 | G,A,C,P,V,L,I,M,F,W, | F,W | S,T,H,N,Q, |
| | S,T,H,N,Q,D,E,K,R | | D,E,K,R |
| V59 | G,A,C,P | M,F,Y,W,H,Q,E,K,R | S,T,Y,H,N,Q, |
| | | | D,E,K,R |
| V61 | G,A,C,P | M,F,Y,W,H,Q,E,K,R | S,T,Y,H,N,Q, |
| | | | D,E,K,R |
| G80 | A,C,P,V | A,C,P,V,L,I,M,F,Y,W, | S,T,Y,H,N,Q, |
| | | S,T,H,N,Q,D,E,K,R | D,E,K,R |
| F81 | G,A,C,P,V,L,I,M,Y,W, | Y,W | S,T,Y,H,N,Q, |
| | S,T,H,N,Q,D,E,K,R | | D,E,K,R |
| G82 | A,C,P,V | A,C,P,V,L,I,M,F,Y,W, | S,T,Y,H,N,Q, |
| | | S,T,H,N,Q,D,E,K,R | D,E,K,R |
| W89 | G,A,C,P,V,L,I,M,F,Y, | F,Y | S,T,Y,H,N,Q, |
| | W,S,T,H,N,Q,D,E,K,R | | D,E,K,R |
| L92 | G,A,C,P,V,I,M | I,M,F,Y,W,K,R | S,T,Y,H,N,Q, |
| | | | D,E,K,R |
| I94 | G,A,C,P,V,L,M | L,M,F,Y,W,K,R | S,T,Y,H,N,Q, |
| | | | D,E,K,R |

Alternative/additional mutations at the positions interacting with the starter-molecule derived region of the substrate (pentyl-group for OLA production) are predicted to increase production of analog products by OAC.

### Example 3: Library Constructs and Strains

Mutant variants of OAC can be constructed as libraries on plasmid by single-site and multi-site (combinatorial) mutagenesis methods, using specific primers at the positions undergoing mutagenesis, amplifying fragments via PCR, and circularizing plasmid via Gibson ligation. A compressed-codon approach can be used to eliminate codon redundancy to lower library size. Plasmid used can be pZS* vector (Novagen), with expression of the olivetol synthase gene under control of a pA1 promoter and lac operator. The resulting OAC proteins will include a fusion to a 6× Histidine tag at the N-terminus. Active variants can be identified by activity assay described below and will be sequenced. Plasmids harboring the mutant libraries of OAC genes may be transformed into E. coli and plated onto agar plates with suitable antibiotic selection.

### Cell Culture for Screening Homologs and Mutant Libraries

From both mutant library transformants and control transformants, single colonies may be picked for growth into 96-well plates using Luria Bertani (LB) growth medium with suitable antibiotic. Following overnight growth, cultures can be sub-cultured into fresh medium of LB with 1% glucose and antibiotic. After 4 hours growth, gene expression may be induced by addition of IPTG, and cells can be pelleted after overnight growth at 30° C., and media discarded. Cells pellets can be stored at −20° C. until ready for assay. Number of samples screened can be approximately three times oversampling based on calculation of total possible variants.

### Example 4: High-Throughput Activity Assay

Cell pellets may be thawed, and subjected to chemical lysis by B-PERII reagent in the presence of protease inhibitor cocktail, 10 mM DTT, benzonase, and lysozyme. Assays can be performed in 96-well plates in a total volume of 40 μl in 50 mM HEPES, pH 7.5 buffer containing 500 μM malonyl-CoA, 500 μM hexanoyl-CoA (Sigma-Aldrich) and 1 μM purified OLS enzyme. Reactions may be initiated by addition of cell lysate then incubated for 30 min, quenched with 75%, of acetonitrile acidified with formic acid, then centrifuged to pellet denatured protein. Supernatants may be transferred to new 96-well plates for LCMS analysis of olivetolic acid, olivetol, PDAT, and HTAL.

### Analytical Analysis of OAC/OLS Reactions

Olivetol, PDAL, OLA, HTAL, CBGA, analogs and combinations thereof may be analyzed by LCMS or LCMS/MS methods using C18 reversed phase chromatography coupled to either Exactive (Thermofisher) or QTrap 4500 (Sciex) mass spectrometers.

Enzymatic reactions, whether conducted in cell lysate or using purified proteins, can be first treated with 6 volumes of organic solvent (acetonitrile containing internal standards) to precipitate proteins, the supernatant can be recovered and further diluted for LCMS analysis, if necessary.

Reversed phase LCMS may be used, and compounds can be identified by their LC retention times and MRM transitions specific to the compounds. LCMSMS analysis can be conducted on Shimadzu UHPLC system coupled with AB Sciex QTRAP4500 mass spectrometer. Agilent Eclipse XDB C18 column (4.6×3.0 mm, 1.8 um) may be used with a 1-min gradient elution at 1 mL/min using water containing 0.1% ammonia acetate as mobile phase A and 90% methanol containing 0.1% ammonia acetate as mobile phase B. The LC column temperature can be maintained at 45° C. Negative ionization mode can be used for all the analytes.

What is claimed is:

1. A non-natural olivetolic acid cyclase (OAC) comprising at least one amino acid variation as compared to a wild type OAC, wherein the non-natural OAC is enzymatically capable of:
a) forming a 2,4-dihydroxy-6-alkylbenzoic acid from a 3,5,7-trioxoacyl-CoA or a 3,5,7-trioxocarboxylate substrate;
b) forming a 2,4-dihydroxy-6-alkylbenzoic acid from a 3,5,7-trioxoacyl-CoA or a 3,5,7-trioxocarboxylate substrate at a greater rate as compared to the wild type OAC;
(c) having a higher affinity for a 3,5,7-trioxoacyl-CoA or a 3,5,7-trioxocarboxylate substrate as compared to the wild type OAC;
(d) with non-rate limiting amount of OLS, forming a 2,4-dihydroxy-6-alkylbenzoic acid from malonyl-CoA and acyl-CoA through a 3,5,7-trioxoacyl-CoA or a 3,5,7-trioxocarboxylate intermediate at a greater rate as compared to the wild type OAC, or
(e) any combination of a), b), c), and d);
with the proviso that the non-natural OAC does not have a single mutation of Y27F relative to SEQ ID NO:1.

2. The non-natural OAC of claim 1, wherein the 3,5,7-trioxoacyl-CoA or a 3,5,7-trioxocarboxylate substrate is 3,5,7-trioxododecyl-CoA or 3,5,7-trioxododecanoate, and wherein the 2,4-dihydroxy-6-alkylbenzoic acid is olivetolic acid.

3. The non-natural OAC of any one of claims 1-2, wherein the 3,5,7-trioxoacyl-CoA or a 3,5,7-trioxocarboxylate substrate is formed by olivetol synthase (OLS) from malonyl-CoA and acyl-CoA, wherein the acyl-CoA is selected from the group consisting of acetyl-CoA, propionyl-CoA, butyryl-CoA, valeryl-CoA, hexanoyl-CoA, heptanoyl-CoA, octanoyl-CoA, nonanoyl-CoA, and decanoyl-CoA.

4. The non-natural OAC of any one of claims 1-3, wherein all or portion of non-natural OAC further comprises all or portion of OLS.

5. The non-natural OAC of any one of claims 3-4, wherein the OLS is a non-natural OLS.

6. The non-natural OAC of claim 5, wherein
(a) the amino acid sequence of the non-natural OLS comprises one or more amino acid substitutions at position(s) selected from the group consisting of: Q82S, P131A, I186F, M187E, M187N, M187T, M187I, M187S, M187A, M187L, M187G, M187V, M187C, S195K, S195M, S195R, S197G, S197V, T239E, K314D, and K314M, corresponding to the amino acid positions of SEQ ID NO:4;
(b) the amino acid sequence of the non-natural OLS comprises two, or more than two amino acid substitutions, selected from: (i) Q82S and P131A, (ii) Q82S and M187S, (iii) Q82S and S195K, (iv) Q82S and S195M, (v) Q82S and S197V, (vi) Q82S and K314D, (vii) P131A and I186F, (viii) P131A and M187S, (ix) P131A and S195M, (x) P131A and S197V, (xi) P131A and K314D, (xii) P131A and K314M, (xiii) I186F and M187S, (xiv) I186F and S195K, (xv) I186F and S195M, (xvi) I186F and T239E, (xvii) I186F and K314D, (xviii) M187S and S195K, (xix) M187S and S195M, (xx) M187S and S197V, (xxi) M187S and T239E, (xxii) M187S and K314D, (xxiii) M187S and K314M, (xxiv) S195K and S197V, (xxv) S195M and S197V, (xxvi) S195M and T239E, (xxvii) S195K and K314D, (xxviii) S195K and K314M, (xxix) S195M and K314D, (xxx) S195M and K314M, (xxxi) S197V and T239E, (xxxii) S197V and K314M, (xxxiii) T239E and K314D, (xxxiv) T239E and K314M, (xxxv) Q82S and I186F, (xxxvi) Q82S and T239E, (xxxvii) Q82S and K314M, (xxxviii) I186F and S197V (xxxix) I186F and K314M, (xl) S195K and T239E, (xli) S197V and K314D, (xlii) P131A and T239E, and (xliii) P131A and S195K corresponding to the amino acid positions of SEQ ID NO:4; or
(c) the amino acid sequence of the non-natural OLS comprises three, or more than three amino acid substitutions, selected from: (i) Q82S, P131A, and I186F, (ii) Q82S, P131A, and M187S, (iii) Q82S, P131A, and S195K, (iv) Q82S, P131A, and S195M, (v) Q82S, P131A, and S197V, (vi) Q82S, P131A, and T239E, (vii) Q82S, P131A, and K314D, (viii) Q82S, P131A, and K314M, (ix) Q82S, I186F, and M187S, (x) Q82S, I186F, and S195M, (xi) Q82S, I186F, and S197V, (xii) Q82S, I186F, and T239E, (xiii) Q82S, I186F, and K314D, (xiv) Q82S, I186F, and K314M, (xv) Q82S, M187S, and S195K, (xvi) Q82S, M187S, and S195M, (xvii) Q82S, M187S, and S197V, (xviii) Q82S, M187S, and T239E, (xix) Q82S, M187S, and K314D, (xx) Q82S, M187S, and K314M, (xxi) Q82S, S195K, and S197V, (xxii) Q82S, S195M, and S197V, (xxiii) Q82S, S195K, and K314D, (xxiv) Q82S, S195K, and K314M, (xxv) Q82S, S195M, and K314D, (xxvi) Q82S, S195M, and K314M, (xxvii) Q82S, S197V, and T239E, (xxviii) Q82S, S197V, and K314D, (xxix) Q82S, S197V, and K314M, (xxx) Q82S, T239E, and K314D, (xxxi) Q82S, T239E, and K314M, (xxxii) P131A, I186F, and M187S, (xxxiii) P131A, I186F, and S195K, (xxxiv) P131A, I186F, and S195M, (xxxv) P131A, I186F, and S197V, (xxxvi) P131A, I186F, and K314D, (xxxvii) P131A, I186F, and K314M, (xxxviii) P131A, M187S, and S195K, (xxxix) P131A, M187S, and S195M, (xl) P131A, M187S, and S197V, (xli) P131A, M187S, and T239E, (xlii) P131A, M187S, and K314D, (xliii) P131A, S195M, and S197V, (xliv) P131A, S195M, and T239E, (xlv) P131A, S195K, and K314D, (xlvi) P131A, S195K, and K314M, (xlvii) P131A, S195M, and K314D, (xlviii) P131A, S195M, and K314M, (xlix) P131A, S197V, and T239E, (1) P131A, S197V, and K314D, (li) P131A, S197V, and K314M, (lii) P131A, T239E, and K314D, (liii) P131A, T239E, and K314M, (liv) I186F, M187S, and S195K, (lv) I186F, M187S, and S195M, (lvi) I186F, M187S, and S197V, (lvii) I186F, M187S, and K314M, (lviii) I186F, S195K, and S197V, (lix) I186F, S195M, and S197V, (lx) I186F, S195K, and T239E, (lxi) I186F, S195M, and T239E, (lxii) I186F, S195K, and K314D, (lxiii) I186F, S195K, and K314M, (lxiv) I186F, S195M, and K314D, (lxv) I186F, S195M, and K314M, (lxvi) I186F, S197V, and T239E, (lxvii) I186F, S197V, and K314D, (lxviii) I186F, S197V, and K314M, (lxix) I186F, T239E, and K314M, (lxx) M187S, S195K, and S197V, (lxxi) M187S, S195M, and S197V, (lxxii) M187S, S195K, and T239E, (lxxiii) M187S, S195M, and T239E, (lxxiv) M187S, S195K, and K314D, (lxxv) M187S, S195K, and K314M, (lxxvi) M187S, S195M, and K314D, (lxxvii) M187S, S195M, and K314M, (lxxviii) M187S, S197V, and T239E, (lxxix) M187S, S197V, and K314D, (lxxx) M187S, S197V, and K314M, (lxxxi) M187S, T239E, and K314D, (lxxxii) M187S, T239E, and K314M, (lxxxiii) S195K, S197V, and T239E, (lxxxiv) S195M, S197V, and T239E, (lxxxv) S195K, S197V, and K314D, (lxxxvi) S195K, S197V, and K314M, (lxxxvii) S195M, S197V, and K314D, (lxxxviii) S195M, S197V, and K314M, (lxxxix) S195K, T239E, and K314D, (xc) S195K, T239E, and K314M, (xci) S195M, T239E, and K314D, (xcii) S195M, T239E, and K314M, and (xciii) S197V, T239E, and K314M corresponding to the amino acid positions of SEQ ID NO: 4.

7. The non-natural OAC of any one of claims 3-6, wherein all or fragment of one of the two subunit of OAC is fused with all or fragment of OLS.

8. The non-natural OAC of any one of claims 4-7, wherein the OAC protein is fused with the OLS protein through a linker molecule.

9. The non-natural OAC of any one of claims 7-8, wherein the N-terminus of the OAC protein is fused with the N-terminus of the OLS protein.

10. The non-natural OAC of any one of claims 7-8 wherein the C-terminus of the OAC protein is fused with the C-terminus of the OLS protein.

11. The non-natural OAC of any one of claims 7-8, wherein the N-terminus of the OAC protein or a fragment thereof is fused with the C-terminus of the OLS protein or a fragment thereof.

12. The non-natural OAC of any one of claims 7-8, wherein the C-terminus of the OAC protein or a fragment thereof is fused with the N-terminus of the OLS protein or a fragment thereof.

13. The non-natural OAC of any one of claims 1-12, wherein the OAC is enzymatically capable of forming olivetolic acid, its analogs and derivatives or a combination thereof at a rate of least two-fold greater as compared to the rate with wild type OAC.

14. The non-natural OAC of claim 13, wherein the OAC is enzymatically capable of forming olivetolic acid, its analogs and derivatives, or a combination thereof from malonyl-CoA and an acyl-CoA in the presence of non-rate limiting amount of OLS at a rate of least two-fold greater as compared to the rate with wild type OAC in the presence of non-rate limiting amount of OLS.

15. The non-natural OAC of any of claims 1-14 comprising at least two amino acid variations as compared to a wild type OAC.

16. The non-natural OAC of claim 15 comprising at least three, four, five, six, seven, eight, nine, or more amino acid variations as compared to a wild type OAC.

17. The non-natural OAC of any of claims 1-16, wherein the amino acid sequence of the non-natural OAC has at least about 45%, about 50%, about 60%, about 65%, about 70%, about 75%, about 80%, about 85%, or about 90% or greater sequence identity to any one of SEQ ID NOs: 1-3 or to at least 25 contiguous amino acids of any one of SEQ ID NOs: 1-3.

18. The non-natural OAC of claim 16 having about 90% or greater identity to any one of SEQ ID NOs: 1-3 or to at least 25 contiguous amino acids of any one of SEQ ID NOs:1-3.

19. The non-natural OAC of any of claims 1-18 comprising one or more amino acid variations at position(s) selected from the group consisting of H5X¹, wherein X¹ is selected from the group consisting of G,A,C,P,V,L,I,M,F,Y,W,Q,E,K,R, S,T,Y,N, Q,D,E,K, and R; I7X², wherein X² is selected from the group consisting of G,A,C,P, V,L,M,FY,W,K,R,S,T,H,N,Q,D, and E; L9X³, wherein X³ is selected from the group consisting of G,A,C,P,V,I,M,F,Y,W,K,R,S,T,Y,H,N,Q,D,E,K,and R; F23X⁴, wherein X⁴ is selected from the group consisting of G,A,C,P,V,L, I,M,Y,W,S,T,H,N,Q,D,E,K, and R; F24X⁵, wherein X⁵ is selected from the group consisting of G,A,C,P,V,I,M,Y,S,T,H,N,Q,D,E,K,R, and W; Y27X⁶, wherein X⁶ is selected from the group consisting of G,A,C,P,V,L,I,M,F,W,S,T,H,N,Q,D,E,K, and R; V59X⁷, wherein X⁷ is selected from the group consisting of G,A,C,P,L,I,M,F,Y,W,H,Q,E,K, and R; V61X⁸, wherein X⁸ is selected from the group consisting of G,A,C,P,L,I,M, F,Y,W,H,Q,E,K,R,S,T,N, and D; V66X⁹, wherein X⁹ is selected from the group consisting of G,A,C,P,L,I,M,F,Y, and W; E67X¹⁰, wherein X¹⁰ is selected from the group consisting of G,A,C,P,V,L,I,M,F,Y, and W; I69X¹, wherein X¹¹ is selected from the group consisting of G,A,C,P,V,L,M,F,Y, and W; Q70X², wherein X² is selected from the group consisting of S,T,H,N,D,E,R,K, and Y; 173X¹³, wherein X¹³ is selected from the group consisting of G,A,C,P,V,L,M,F,Y, and W; I74X¹⁴, wherein X¹⁴ is selected from the group consisting of G,A,C,P,V,L,M,F,Y, and W; V79X¹, wherein X¹⁵ is selected from the group consisting of G,A,C,P,L,I,M,F,Y, and W; G80X¹⁶, wherein X¹⁶ is selected from the group consisting of A,C,P,V,L,I,M,F,Y,W,S,T,H,N,Q,D,E, K, and R; F81X¹⁷, wherein X¹⁷ is selected from the group consisting of G,A,C,P,V,L,I,M,Y,W,S,T,H,N,Q,D,E,R, and K; G82X¹⁸, wherein X¹⁸ is selected from the group consisting of A,C,P,V,L,I,M,F,Y,W,S,T,H,N,Q,E,K, and R; D83X¹⁹, wherein X¹⁹ is selected from the group consisting of S,T,H,Q,N,E,R,K, and Y; R86X²⁰, wherein X²⁰ is selected from the group consisting of S,T,H,Q, N,D, E,K, and Y; W89X², wherein X² is selected from the group consisting of G,A,C, P,V,L,I,M,F,Y,W,S,T,H,N,Q,D,E,K, and R; L92X²², wherein X²² is selected from the group consisting of G,A,C,P,V,I,M,F,Y, and W; I94X²¹, wherein X²³ is selected from the group consisting of G,A,C,P,V,L,M,F,Y,W,K,R,S,T,Y,H,N,Q,D, and E; D96X²⁴, wherein X²⁴ is selected from the group consisting of S,T,H,Q,N,E,R,K, and Y; V46X²⁵, wherein X²⁵ is selected from the group consisting of G,A,C,P, L,I,M,F,Y, and W; T47X²⁶, wherein X²⁶ is selected from the group consisting of S,H,Q,N,D,E,R,K, and Y; Q48X²⁷, wherein X²⁷ is selected from the group consisting of S,T,H,N,D,E,R,K, and Y; K49X²⁸, wherein X²⁸ is selected from the group consisting of S,T,H,Q,N,D,E,R, and Y; N50X²⁹, wherein X²⁹ is selected from the group consisting of G,A,C,P,V,L,I,M,F,Y, and W; and K51X³⁰, wherein X³⁰ is selected from the group consisting of S,T,H,Q,N,D,E,R, and Y; V46*X³, wherein X³¹ is selected from the group consisting of G,A,C,P,L,I,M,F,Y, and W; T47*X³², wherein X³² is selected from the group consisting of S,H,Q,N,D,E,R,K, and Y; Q48*X³³, wherein X³³ is selected from the group consisting of S,T,H,N,D,E,R,K, and Y; K49*X³⁴, wherein X³⁴ is selected from the group consisting of S,T,H,Q,N, D,E,R, and Y; N50*X³⁵, wherein X³⁵ is selected from the group consisting of G,A,C,P,V,L,I,M,F,Y, and W; and K51*X³⁶, wherein X³⁶ is selected from the group consisting of S,T,H,Q,N,D,E,R, and Y, wherein the amino acid positions correspond to SEQ ID NO: 1, and wherein
the non-natural OAC is not a single variant of K4A, H5A, H5L, H5Q, H5S, H5N, H5D, 17L, 17F, L9A, L9W, K12A, F23A, F23I, F23W, F23L, F24L, F24W, F24A, Y27F, Y27M, Y27W, V28F, V29M, K38A, V40F, D45A, H57A, V59M, V59A, V59F, Y72F, H75A, H78A, H78N, H78Q, H78S, H78D, or D96A, and wherein the “*” indicates amino acid residues from chain B of OAC dimer and corresponding to SEQ ID NO: 1.

20. The non-natural OAC of claim 19 comprising one or more amino acid variations at position(s) selected from the group consisting of: L9, F23, V59, V61, V66, E67, I69, Q70, I73, I74, V79, G80, F81, G82, D83, R86, W89, L92, I94, V46, T47, Q48, K49, N50, K51, V46*, T47*, Q48*, K49*, N50*, and K51*, wherein the amino acid positions correspond to SEQ ID NO: 1.

21. The non-natural OAC of any one of claims 1-19, wherein the amino acid sequence of the OAC comprises SEQ ID NO: 2.

22. The non-natural OAC of any one of claims 1-21
having a higher affinity for a 3,5,7-trioxoacyl-CoA or a 3,5,7-trioxocarboxylate substrate that is different than 3,5,7 trioxododecanoyl-CoA, as compared to the wild type OAC, and/or
that is able to form a 3,5,7-trioxoacyl-CoA or a 3,5,7-trioxocarboxylate substrate that is different than 3,5,7 trioxododecanoyl-CoA at a greater rate as compared to the wild type OAC.

23. The non-natural OAC of any one of claims 1-22, wherein the non-natural OAC having a higher affinity for a 3,5,7-trioxoacyl-CoA or a 3,5,7-trioxocarboxylate substrate that is larger and more hydrophobic than 3,5,7 trioxododecanoyl-CoA, comprising one or more amino acid variations at position(s): H5X¹, wherein X¹ is selected from the group consisting of G,A,C,P,V;17X², wherein X² is selected from the group consisting of G,A,C,P,V,L, and M; L9X³, wherein X³ is selected from the group consisting of G,A,C,P,V,I, and M; F23X⁴, wherein X⁴ is selected from the group consisting of G,A,C,P,V,L,I,M,Y,W,S,T,H,N, Q,D,E,K, and R; F24X⁵, wherein X⁵ is selected from the group consisting of G,A,C, P,V,L,I,M,Y,W,S,T, H,N,Q,D,E,K, and R; Y27X⁶, wherein X⁶ is selected from the group consisting of G,A,C,P,V, L,I,M,F,W,S,T,H,N,Q,D, E,K, and R; V59X⁷, wherein X⁷ is selected from the group consisting of G,A,C, and P; V61X⁸, wherein X⁸ is selected from the group consisting of G,A,C, and P; G80X¹⁶, wherein X¹⁶ is selected from the group consisting of A,C,P,V,L,I,M,F,Y,W,S,T,H,N,Q,D,E, K, and R; F81X¹⁷, wherein X¹⁷ is selected from the group consisting of Y and W; G82X¹⁸, wherein X¹⁸ is selected from the group consisting of A,C,P,V,L,I,M,F,Y,W,S,T,H,N, Q,D,E,K, and R; W89X², wherein X² is selected from the group consisting of G,A,C,P,V,L,I,M,F, Y,W,S, T,H,N,Q,D,E,K, and R; L92X²², wherein X²² is selected from the group consisting of G,A,C,P, V,I, and M; and I94X²³, wherein X²³ is selected from the group consisting of G,A,C,P,V,L, and M, wherein the amino acid positions correspond to SEQ ID NO: 1.

24. The non-natural OAC of any one of claims 1-22, wherein the non-natural OAC having a higher affinity for a 3,5,7-trioxoacyl-CoA or a 3,5,7-trioxocarboxylate substrate that is smaller and less hydrophobic than 3,5,7 trioxododecanoyl-CoA, comprising one or more amino acid variations at position(s): H5X¹, wherein X¹ is selected from the group consisting of V,M,F,Y,W,Q,E, and K, and R; I7X², wherein X² is selected from the group consisting of L,M,F,Y,W,K, and R; L9X³, wherein X³ is selected from the group consisting of I,M,F,Y,W,K, and R; F23X⁴, wherein X⁴ is selected from the group consisting of Y and W; F24X⁵, wherein X⁵ is selected from the group consisting of Y and W; Y27X⁶, wherein X⁶ is selected from the group consisting of F and W; V59X⁷, wherein X⁷ is selected from the group consisting of M,F,Y,W,H,Q,E,K, and R; V61X⁸, wherein X⁸ is selected from the group consisting of M,F,Y,W,H,Q,E,K, and R; G80X¹⁶, wherein X¹⁶ is selected from the group consisting of A,C,P, and V; F81X¹⁷, wherein X¹⁷ is selected from the group consisting of G,A,C,P,V,L,I,M,Y,W,S,T,H,N,Q,D,E,K, and R; G82X¹⁸, wherein X¹⁸ is selected from the group consisting of A,C,P, and V; W89X¹, wherein X² is selected from the group consisting of F, and Y; L92X², wherein X² is selected from the group consisting of I,M,F,Y,W,K, and R; and I94X²³, wherein X²³ is selected from the group consisting of L,M,F,Y,W,K, and R, wherein the amino acid positions correspond to SEQ ID NO: 1.

25. The non-natural OAC of any one of claims 1-22, wherein the non-natural OAC having a higher affinity for a 3,5,7-trioxoacyl-CoA or a 3,5,7-trioxocarboxylate substrate that is more polar and/or more charged than 3,5,7 trioxododecanoyl-CoA, comprising one or more amino acid variations at position(s): H5X¹, wherein X¹ is selected from the group consisting of S,T,Y,N,Q,D,E,K, and R; I7X², wherein X² is selected from the group consisting of S,T,Y,H,N,Q,D,E,K, and R; L9X³, wherein X³ is selected from the group consisting of S,T,Y,H,N,Q,D,E,K, and R; F23X⁴, wherein X⁴ is selected from the group consisting of S,T,Y,H,N,Q,D, E,K, and R; F24X⁵, wherein X⁵ is selected from the group consisting of S,T,Y,H,N,Q,D,E,K, and R; Y27X⁶, wherein X⁶ is selected from the group consisting of S,T,H,N,Q,D,E,K, and R; V59X⁷, wherein X⁷ is selected from the group consisting of S,T,Y,H,N,Q,D, E,K, and R; V61X⁸, wherein X is selected from the group consisting of S,T,Y,H,N, Q,D,E,K, and R; G80X¹⁶, wherein X¹⁶ is selected from the group consisting of S,T, Y,H,N,Q,D,E,K, and R; F81X¹⁷, wherein X¹⁷ is selected from the group consisting of S,T,Y,H,N,Q,D,E,K, and R; G82X¹⁸, wherein X¹⁸ is selected from the group consisting of S,T,Y,H,N,Q,D,E,K, and R; W89X², wherein X²¹ is selected from the group consisting of S,T,Y,H,N,Q,D,E,K, and R; L92X²², wherein X²² is selected from the group consisting of S,T,Y,H,N,Q,D,E,K, and R; and I94X²³, wherein X²³ is selected from the group consisting of S,T,Y,H,N,Q,D,E,K, and R, wherein the amino acid positions correspond to SEQ ID NO: 1.

26. The non-natural OAC of any of claims 3-25, wherein the amino acid sequence of OLS is at least 60% identical to at least 25 or more contiguous amino acids of SEQ ID NO: 4.

27. The OLS of claim 26, wherein the OLS comprises one or more amino acid substitutions at position(s) selected from the group consisting of: Q82S, P131A, I186F, M187E, M187N, M187T, M187I, M187S, M187A, M187L, M187G, M187V, M187C, S195K, S195M, S195R, S197G, S197V, T239E, K314D, and K314M, corresponding to the amino acid positions of SEQ ID NO: 4.

28. A nucleic acid encoding the non-natural OAC of any one of claims 1-27.

29. An expression construct comprising the nucleic acid of claim 28, wherein the nucleic acid encoding the non-natural OAC is operably linked to a regulatory element, wherein the regulatory element is heterologous to the OAC.

30. An engineered cell comprising the non-natural OAC of any one of claims 1-27, or the nucleic acid of claim 28 or 29.

31. The engineered cell of claim 30, wherein the engineered cell comprises enzymes for the olivetolic acid pathway.

32. The engineered cell of claim 31, wherein the olivetolic acid pathway comprises the non-natural OAC and a natural or non-natural OLS.

33. The engineered cell of claim 32, comprising a non-natural OLS having at least 60% identity to SEQ ID N:4 or to at least 25 or more contiguous amino acids of SEQ ID NO: 4.

34. The engineered cell of claim 33, wherein the non-natural OLS comprises one or more amino acid substitutions at position(s) selected from the group consisting of: Q82S, P131A, I186F, M187E, M187N, M187T, M187I, M187S, M187A, M187L, M187G, M187V, M187C, S195K, S195M, S195R, S197G, S197V, T239E, K314D, and K314M, corresponding to the amino acid positions of SEQ ID NO: 4.

35. The engineered cell of any one of claims 30-34, wherein the engineered cell comprises enzymes for a geranyl pyrophosphate pathway.

36. The engineered cell of claim 35, wherein the geranyl pyrophosphate (GPP) pathway comprises one or more of geranyl pyrophosphate synthase (GPPS), famesyl pyrophosphate synthase, isoprenyl pyrophosphate synthase, geranylgeranyl pyrophosphate synthase.

37. The engineered cell of claim 36, wherein the GPP pathway comprises a mevalonate (MVA) pathway, a non-mevalonate (MEP) pathway, an alternative non-MEP, non MVA geranyl pyrophosphate pathway, or a combination of one or more pathways, wherein the alternative non-MEP, non-MVA geranyl pyrophosphate pathway comprises one or more of the enzymes alcohol kinase, alcohol diphosphokinase, phosphate kinase, isopentenyl diphosphate isomerase, and geranyl pyrophosphate synthase enzymes.

38. The engineered cell of any one of claims 30-37, wherein the engineered cell comprises one or more exogenous nucleic acids, wherein at least one exogenous nucleic acid encodes the non-natural OAC of claims 1-26.

39. The engineered cell of claim 38, wherein the engineered cell comprises two or more exogenous nucleic acids, and wherein at least one exogenous nucleic acid encodes the non-natural OAC, and another exogenous nucleic acid encodes a natural or non-natural OLS.

40. The engineered cell of any one of claims 30-39, wherein the cell is a prokaryote or a eukaryote.

41. The engineered cell of claim 40, wherein the cell is a eukaryote selected from the group consisting of yeast, fungi, microalgae, and algae.

42. The engineered cell of claim 40, wherein the cell is a prokaryote selected from the group consisting of Escherichia, Cyanobacteria, Corynebacterium, Bacillus, Ralstonia, Zymomonas, and Staphylococcus.

43. The engineered cell of any one of claims 30-42, wherein the cell produces olivetolic acid, cannabigerolic acid (CBGA), THCA, CBDA, CBCA, cannabigerol, THC, CBD, CBC, analogs or derivatives thereof, or a combination thereof, wherein the cell produces less olivetol, analogs or derivatives of olivetol, pentyl diacetic acid lactone (PDAL), hexanoyl triacetic acid lactone (HTAL), a lactone analog or derivatives thereof, or a combination thereof as compared to a wild-type non-engineered cell or an engineered cell comprising the wild-type OAC.

44. A cell extract or cell culture medium of the engineered cell of any of claims 30-43 comprising olivetolic acid, cannabigerolic acid (CBGA), CBG, analogs or derivatives thereof, or a combination thereof.

45. The cell extract or cell culture medium of claim 44, wherein the cell extract or cell culture medium comprises olivetolic acid, analogs or derivatives thereof, or a combination thereof, at a concentration of 50% or greater of the total products of non-natural OAC catalyzed reactions.

46. The cell extract or cell culture medium of any one of claim 44 or 45, wherein the cell extract or cell culture medium comprises cannabigerolic acid (CBGA), CBG, analogs or derivatives thereof, or a combination thereof, and wherein the cell extract or cell culture medium comprises olivetol or its analogs, pentyl diacetic acid lactone (PDAL), hexanoyl triacetic acid lactone (HTAL), or lactone analog or derivatives thereof, or a combination thereof.

47. The cell extract of claim 46, wherein olivetol or its analogs, pentyl diacetic acid lactone (PDAL), hexanoyl triacetic acid lactone (HTAL), or lactone analog or derivatives thereof, or a combination thereof is present at a concentration of no more than about 50% to about 0.1% by weight of the cell extract or cell culture medium.

48. A method for forming an aromatic compound, comprising:
(a) contacting an acyl-CoA and malonyl-CoA substrates with an OLS to form a polyketide, or analog or derivative thereof
(b) contacting the polyketides, or analog or derivative thereof with an OAC enzyme of any of claims 1-26, wherein the contacting forms the aromatic compound.

49. The method of claim 48, wherein the aromatic compound is olivetolic acid, analogs and derivatives thereof, or combinations thereof.

50. A method for forming a 2,4-dihydroxy-6-alkylbenzoic acid from a 3,5,7-trioxoacyl-CoA or a 3,5,7-trioxocarboxylate substrate, comprising
a) providing a 3,5,7-trioxoacyl-CoA or a 3,5,7-trioxocarboxylate substrate,
b) providing non-natural olivetolic acid cyclase comprising at least one amino acid variation as compared to a wild type OAC, wherein the non-natural OAC is enzymatically capable of:
i) forming a 2,4-dihydroxy-6-alkylbenzoic acid from a 3,5,7-trioxoacyl-CoA or a 3,5,7-trioxocarboxylate substrate at a greater rate as compared to the wild type OAC;
(ii) having a higher affinity for a 3,5,7-trioxoacyl-CoA or a 3,5,7-trioxocarboxylate substrate; or
both i) and ii),
wherein the non-natural olivetolic acid cyclase is based on SEQ ID NO:1 or an OAC template having at least 60% identity to SEQ ID NO:1 or to at least 25 contiguous amino acids of SEQ ID NO:1, and the at least one amino acid variation is at position H5, I7, L9, F23, F24, Y27, V59, V61, V66, E67, I69, Q70, I73, I74, V79, G80, F81, G82, D83, R86, W89, L92, I94, D96, V46, T47, Q48, K49, N50, K51, V46*, T47*, Q48*, K49*, N50*, and K51*, wherein the “*” indicates residues from chain B of OAC dimer.

51. The method of claim 50, wherein the non-natural olivetolic acid cyclase is any one of non-natural OAC of claims 1-26.

52. The method of any one of claims 50-51, wherein the 2,4-dihydroxy-6-alkylbenzoic acid is olivetolic acid, and wherein the 3,5,7-trioxoacyl-CoA or a 3,5,7-trioxocarboxylate substrate is 3,5,7-trioxododecanoyl-CoA or 3,5,7-trioxododecanoate.

53. The method of any one of claims 50-51, wherein the 3,5,7-trioxoacyl-CoA or a 3,5,7-trioxocarboxylate substrate is not 3,5,7-trioxododecanoyl-CoA or 3,5,7-trioxododecanoate, and wherein the 2,4-dihydroxy-6-alkylbenzoic acid is not olivetolic acid.

54. A method for forming a cannabinoid, an analog or derivatives thereof, or a combination thereof, comprising:
(a) contacting malonyl-CoA and an acyl-CoA substrates with a non-natural OLS that preferentially produces polyketides, analogs, and derivatives thereof, or combinations thereof over olivetol, analogs and derivatives of olivetol, pentyl diacetic acid lactone (PDAL), or lactone analogs and derivatives as compared to the wild type OLS;
(b) contacting the polyketides, analogs and derivatives thereof, or combinations thereof with the OAC of any of claims 1-26, wherein the contacting forms the olivetolic acid, analogs and derivatives thereof, or combinations thereof;
(c) converting the olivetolic acid, analogs and derivatives thereof, or combinations thereof to the cannabinoid, an analog or derivatives thereof, or a combination thereof thermally, chemically or enzymatically, or by a combination thereof.

55. The method of any of claims 48-49 and 54, wherein the molar ratio of malonyl-CoA to acyl-CoA in the range of about 500:1 to about 1:500, about 250:1 to about 1:250, about 150:1, to about 10:1, to about 3:1, to about 1:150, about 100:1 to about 1:100, about 75:1 to about 1:75, about 50:1 to about 1:50, about 25:1 to about 1:25, about 15:1 to about 1:15, or about 10:1 to about 1:10.

56. The method of any of claims 48-49 and 54-55, wherein the acyl-CoA substrate is selected from the group consisting of acetyl-CoA, propionyl-CoA, butyryl-CoA, valeryl-CoA, hexanoyl-CoA, heptanoyl-CoA, octanoyl-CoA, nonanoyl-CoA, and decanoyl-CoA.

57. The method of any of claims 54-56, wherein the cannabinoid is cannabigerolic acid (CBGA), THCA, CBDA, CBCA, cannabigerol, THC, CBD, CBC, analogs or derivatives thereof, or a combination thereof.

58. The method of claim 57, wherein the cannabinoid is CBGA.

59. The method of any of claims 48-58, which occurs in the engineered cell of any one of claims 30-42.

60. The method of any one of claims 54-59, wherein the non-natural OAC enzyme is present in a molar excess over the OLS enzyme.

61. The method of any of claims 54-60 further comprises a step of isolating or purifying the cannabinoid, analogs and derivatives thereof, or combinations thereof from the reaction mixture.

62. The method of claim 61, wherein the step of isolating or purifying comprises one or more of liquid-liquid extraction, pervaporation, evaporation, filtration, membrane filtration, reverse osmosis, nanofiltration, ultrafiltration, microfiltration, membrane filtration with diafiltration, membrane separation, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration.

63. A composition comprising a cannabinoid, analogs, or derivatives thereof, or combinations thereof obtained from the engineered cell of any of claims 30-43, or the method of any of claims 54-62, wherein the composition comprises olivetol or analogs and derivatives of olivetol, pentyl diacetic acid lactone (PDAL), hexanoyl triacetic acid lactone (HTAL), a lactone analog, or a combination thereof at a concentration of no more than about 0.1% to about 0.0001% by weight of the composition.

64. The composition of claim 63, wherein the composition is a cannabinoid, wherein the cannabinoid is cannabigerolic acid (CBGA), THCA, CBDA, CBCA, cannabigerol, THC, CBD, CBC, analogs or derivatives thereof, or a combination thereof.

65. The composition of claim 63 or 64, comprising CBGA, CBG, analogs or derivatives thereof at a concentration of 60% or greater of total cannabinoid compound(s) in the composition.

66. The composition of any one of claims 63-65, further comprising at least one pharmaceutically acceptable excipient selected from the group consisting of a diluent, a binder, a lubricant, a disintegrant, a flavoring agent, a coloring agent, a stabilizer, a surfactant, a glidant, a plasticizer, a preservative, an essential oil, a humectant, an absorption accelerator, a wetting agent, an absorber, and a buffering agent.

67. The composition of any one of claims 63-66, wherein the composition is a pharmaceutical, an edible, personal care product, or a cosmetic.


SEQUENCE LISTING (4 sequences)

SEQ ID NO: 1 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 2 - protein, 101 aa, Artificial Sequence
  OAC homolog
MAVKHLIVLKFKEDITEAQKDEFFKTYVNLVNIIPAMKEVYWGKDVTAKNKDEGYTHIVEVTFESVETIQEYISHPAHVGFGDVYRSFWEKLLIFDYTPTK

SEQ ID NO: 3 - protein, 109 aa, Cicer arietinum
MAETKETVKHIVIGKFKDEISEERIDELIKGYANLVNLIPTMKSFHWGKDVSGENLHQGYTHVFESTFESVEGIAEYVAHPTHVEYANLLLPSLEKVLIIDYKPTFVNL

SEQ ID NO: 4 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY